SEQUENCE LISTING (20 sequences)

SEQ ID NO: 1 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 270 aa, Artificial Sequence
  Recombinant Human Complement Factor H protein Complement Control Protein Domains 1-4 and C- terminal 6x His-tagged
EKREAEAAGEQKLISEEDLEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEHHHHHH

SEQ ID NO: 3 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 4 - protein, 317 aa, Homo sapiens
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVAQEETEILTADMDAERRRIKSLLPKLSCGVKNRMHI

SEQ ID NO: 5 - protein, 244 aa, Homo sapiens
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 6 - protein, 4 aa, Homo sapien
RRKR

SEQ ID NO: 7 - protein, 583 aa, Artificial Sequence
  Recombinant Precursor Human Complement Factor I comprising an S525A mutation
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDAGGPLVCMDANNVTYVWGVVSWGENCGKPEFPGVYTKVANYFDWISYHVGRPFISQYNV

SEQ ID NO: 8 - protein, 244 aa, Artificial Sequence
  Complement Factor I light chain derived from precursor human complement factor I comprising an S525A mutation
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDAGGPLVCMDANNVTYVWGVVSWGENCGKPEFPGVYTKVANYFDWISYHVGRPFISQYNV

SEQ ID NO: 9 - protein, 1663 aa, Homo sapien
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 10 - protein, 2039 aa, Homo sapien
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 11 - protein, 1930 aa, Artificial Sequence
  Recombinant Soluble Complement Receptor 1 lacking the transmembrane domain sequence (amino acids 1972-2039) and lacking the signal peptide (amino acids 1-41)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHD

SEQ ID NO: 12 - protein, 392 aa, Homo sapien [sequence omitted]

SEQ ID NO: 13 - protein, 358 aa, Artificial Sequence
  Recombinant Human Mature Membrane Cofactor Protein lacking the signal peptide (amino acids 1-34)
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 14 - protein, 449 aa, Homo sapien
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 15 - protein, 431 aa, Artificial Sequence
  Recombinant Factor H-like protein 1 lacking the signal peptide (amino acids 1-18)
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 16 - protein, 583 aa, Artificial Sequence
  Recombinant Precursor Human Complement Factor I comprising a H380R mutation
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAARCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 17 - protein, 244 aa, Artificial Sequence
  Complement Factor I Light Chain (LC) derived from precursor Human Complement Factor I comprising an H380R mutation
IVGGKRAQLGDLPWQVAIKDASGITCGGIYIGGCWILTAARCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 18 - protein, 1744 aa, Homo sapien
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 19 - protein, 597 aa, Homo sapien
MHPPKTPSGALHRKRKMAAWPFSRLWKVSDPILFQMTLIAALLPAVLGNCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEPNSCINLPDIPHASWETYPRPTKEDVYVVGTVLRYRCHPGYKPTTDEPTTVICQKNLRWTPYQGCEALCCPEPKLNNGEITQHRKSRPANHCVYFYGDEISFSCHETSRFSAICQGDGTWSPRTPSCGDICNFPPKIAHGHYKQSSSYSFFKEEIIYECDKGYILVGQAKLSCSYSHWSAPAPQCKALCRKPELVNGRLSVDKDQYVEPENVTIQCDSGYGVVGPQSITCSGNRTWYPEVPKCEWETPEGCEQVLTGKRLMQCLPNPEDVKMALEVYKLSLEIEQLELQRDSARQSTLDKEL

SEQ ID NO: 20 - protein, 252 aa, Homo sapien
MFFWCACCLMVAWRVSASDAEHCPELPPVDNSIFVAKEVEGQILGTYVCIKGYHLVGKKTLFCNASKEWDNTTTECRLGHCPDPVLVNGEFSSSGPVNVSDKITFMCNDHYILKGSNRSQCLEDHTWAPPFPICKSRDCDPPGNPVHGYFEGNNFTLGSTISYYCEDRYYLVGVQEQQCVDGEWSSALPVCKLIQEAPKPECEKALLAFQESKNLCEAMENFMQQLKESGMTMEELKYSLELKKAELKAKLL

(19) United States
(12) Patent Application Publication
(10) Pub. No.: US 2024/0255503 A1
(43) Pub. Date: Aug. 1, 2024

(54) SCREENING FOR THE EFFECTS OF COMPLEMENT PROTEIN CHANGES

(71) Applicant: UNIVERSITY OF NEWCASTLE UPON TYNE, Tyne and Wear (GB)

(72) Inventors: Thomas HALLAM, Tyne and Wear (GB); Kevin MARCHBANK, Tyne and Wear (GB); David KAVANAGH, Tyne and Wear (GB); Claire HARRIS, Tyne and Wear (GB)

(21) Appl. No.: 18/565,377

(22) PCT Filed: May 25, 2022
(86) PCT No.: PCT/GB2022/051308

§ 371 Date: Nov. 29, 2023

(30) Foreign Application Priority Data
May 31, 2021 (GB) 2107754.0

(51) Int. Cl. G01N 33/564; C07K 14/47; G01N 33/543

(52) U.S. Cl. CPC G01N 33/564; C07K 14/472; G01N 33/54373; G01N 2333/4716

(57) ABSTRACT

Aspects of the present invention relate to the detection of binding events associated with a protein complex comprised of a protease deficient Complement Factor I (FI) protein. Certain embodiments of the present invention provide for a protein complex comprising a Complement Factor I, C3b and further comprising at least one other protein from Complement Factor H (FH), Factor-H-like protein 1 (FHL-1), soluble membrane cofactor protein (sMCP), and soluble complement receptor 1 (sCR1), wherein the Complement Factor I comprises at least one mutation that causes inactivation of Complement Factor I protease activity. Further embodiments of the present invention provide for a protein complex comprising a Complement Factor I, C4b and further comprising at least one other protein from soluble complement receptor 1 (sCR1), soluble membrane cofactor protein (sMCP) and C4b binding protein (C4bp), wherein the Complement Factor I comprises at least one mutation that causes inactivation of Complement Factor I protease activity.

### TECHNICAL FIELD OF THE INVENTION

Aspects of the present invention relate to the detection of binding events associated with a protein complex comprised of a protease deficient Complement Factor I (FI) protein. Certain embodiments of the present invention provide for a protein complex comprising a Complement Factor I (FI), C3b and further comprising at least one other protein from Complement Factor H (FH), Factor-H-like protein (FHL-1), soluble membrane cofactor protein (sMCP), and soluble complement receptor 1 (sCR1), wherein the Complement Factor I comprises at least one mutation that causes inactivation of Complement Factor I protease activity. Further embodiments of the present invention provide for a protein complex comprising a Complement Factor I, C4b and further comprising at least one other protein from soluble complement receptor 1 (sCR1), soluble membrane cofactor protein (sMCP) and C4b binding protein (C4 bp), wherein the Complement Factor I comprises at least one mutation that causes inactivation of Complement Factor I protease activity.

### BACKGROUND TO THE INVENTION

The complement system is a part of the innate immune system, which is comprised of a large number of discrete plasma proteins that react with one another to opsonize pathogens and induce a series of inflammatory responses during infection. Complement system proteins are subject to extensive and complex regulatory mechanisms, and once activated influence a broad range of metabolic signal cascades. Indeed, multiple complex interactions exist between the complement system and the coagulation and fibrinolytic cascades. Thus, unsurprisingly, aberrant complement system function is implicated in a broad spectrum of clinical disorders—ranging from common diseases, such as age-related macular degeneration (AMD) and Alzheimer's Disease (AD), to rare diseases, such as paroxysmal nocturnal hemoglobinuria (PNH), atypical haemolytic uremic syndrome (aHUS) and C3 glomerulopathy (C3G).

Assessment of complement system pathway function in patients involves comprehensive laboratory analysis involving; analysis of the activation states of the different pathways, determining the presence of autoantibodies, determination of the concentration and function of single components and regulators, and molecular analysis of complement genes. Moreover, development of therapeutics for complement related disorders offers several challenges. The large quantity of circulating complement system proteins offers substantial pharmacokinetic and pharmacodynamic problems in identifying an effective therapeutic regimen. Additionally, the central role of complement system protein in fighting infection demands high therapeutic specificity to avoid undesirable immunological side-effects. Furthermore, the intricacy of the complement system often confounds identification of an appropriate target for intervention.

The key mechanism for complement system regulation is proteolytic cleavage of the central complement component; complement component 3 (C3). The alternative pathway of the complement system (AP) is continually activated by a tick-over mechanism and can also be triggered by the classical and lectin pathways. In the AP, C3 undergoes spontaneous hydrolysis, depositing C3b onto the surface of foreign and host cells in the vicinity. On an activating surface such as a bacterium, C3b joins with Factor B, which then is cleaved by Factor D to form the C3 convertase, C3bBb. The binding of properdin stabilizes this enzyme. This enzyme complex then cleaves more C3 to C3b to initiate a feedback loop. Downstream of this amplification loop, C3b may also join with the C3 convertase to form the C5 convertase. C5 is cleaved to the anaphylatoxin C5a and C5b, which initiates formation of the lytic membrane attack complex (C5b-9).

Within the AP, complement factor H (FH) acts as a cofactor for complement factor I (FI). FI is a serine protease that cleaves C3b into its inactive form—iC3b. Thus, FH and FI act to inhibit the alternative pathway of the complement system. For inactivation of C3b to occur, FI and FH must associate with C3b to form a transient tri-molecular complex (TMC) to facilitate proteolytic cleavage of C3b to iC3b. Alternatively, FHL-1, sMCP and sCR1 may function as cofactors to recruit FI, replacing FH to form a different TMC. The presence of de novo mutations in CD46 (MCP), CD35 (CR1), CFH (which translates to FH and the alternatively spliced molecule FHL-1), CFI and C3 (C3b) may affect the respective binding affinity of the respective TMC, and therefore effect proteolytic inactivation of C3b. Aberrant AP regulatory TMC formation can result in dysregulation of the complement system.

Within the classical pathway, complement component C4 is cleaved into complement components C4a and C4b by activated C1s. C4b then covalently binds, either via an ester or an amide linkage, to the surface of host or foreign cells in the vicinity. Activated C1s also cleaves C2 that binds to C4b, leading to the formation of the C4bC2a complex, which is the C3 convertase of the classical pathway. Proteolytic inactivation of C4b is therefore a step in regulation of the classical complement pathway. C4 bp is the main soluble inhibitor of the classical pathway, C4 bp serving as cofactor in factor I-mediated proteolysis of cell-bound and soluble C4b. CR1 protein is also known to serve as a factor I cofactor in factor I mediated proteolysis of C4b.

Dysregulation of the complement system is known to mediate several disorders. For example, heterozygous mutations in CFI have been associated with a predisposition to atypical hemolytic uremic syndrome, a disease characterized by acute renal failure, microangiopathic hemolytic anemia and thrombocytopenia. Recently, haploinsufficiency in circulating FI has been identified in individuals with very rare CFI variants and these mutations have been identified in heterozygous individuals with atypical hemolytic uremic syndrome (aHUS) and have been strongly associated with advanced Age-Related Macular Degeneration (AMD), supporting the role of CFI in risk of AMD (Kavanagh et al., Human Molecular Genetics, 2015, Vol. 24, No. 13 3861-3870; Hallam et al., Investigative Ophthalmology & Visual Science, 2020; 61(6):18). AMD is the most common cause of vision loss in individuals over the age of 50 and currently there are few treatment options. Disruption of FI mediated complement system regulation has also been implicated in the progression of early stage Alzheimer's disease (Hakobyan et al, Journal of Alzheimer's Disease, 54; 2, 707-716, 2016). Complete functional FI deficiency has also been recently identified in a patient with fulminant cerebral inflammation and CFI variants in compound heterozygosity (Altmann et al, Neurolology, Neuroimmunology, andNeuroinflammation, 2020; 7:e689). Complete FI deficiency caused by homozygous or compound heterozygous mutations in CFI has historically been associated with recurrent infection by encapsulated microorganisms leading to sepsis (Alba-Dominguez et al., Orphanet Journal of Rare Diseases 2012, 7:42). Polymorphisms and rare genetic variants in CFH have also been implicated in the progression of AMD, Schizophrenia, aHUS and C3G, and complete deficiency in FH results in patients being at risk of pyogenic infection and renal disease.

Genome wide association studies (GWAS) have indicated that patients with AMD often have genetic variants in complement system proteins. Specifically, GWAS have indicated genetic variants in CFI, CFH, and C3 are major risk factors for AMD. Genetic variants in CFI and CFH can lead to dysfunction in FI and FH protein resulting in overactivation of the AP, resulting in systemic inflammation. This process occurring in the delicate tissues of the eye is thought contribute to the progression of AMD.

Therapeutic strategies based on supplemental FI/FH may have differing efficacy depending upon the patients' genotype. Indeed, patients with genetic variants causing dysfunction in FHL-1, sMCP, sCR1, FI, FH and C3 (C3b) may benefit from personalized therapeutics. However, assays for characterizing the impact of de novo FHL-1, sMCP, sCR1, FI, FH, and C3 (C3b) variants are limited. Thus, a rapid and sensitive screening tool is required to assess drug/protein binding and function.

Regulators of complement activity (such as FH, sMCP and CR1) comprise modular complement-control-protein (CCP) domains. Structures of C3b in complex with three to four consecutive CCP domains from several regulators showed that the regulators share an extended binding platform on C3b.

Xue et al; Nature Structural & Molecular Biology; 24; 643-651, discloses a crystal structure of C3b complexed with a short variant of human FH protein comprised of CCP domains 1-4, and a catalytically inactive FI comprising an “S507A” mutation. Although the molecular architecture of a related TMC complex is disclosed therein, the data fails to offer real time resolution of AP regulatory TMC binding and formation, or the effect of mutations on TMC assembly kinetics.

A relevant method for characterizing AP regulatory TMC formation and related kinetic events in real time has not yet been described in the art.

It is therefore an aim of certain embodiments of the present invention to mitigate at least some of the problems of the prior art.

It is an aim of certain embodiments of the invention to provide a method for characterizing the formation of an AP regulatory TMC or a related complex in real time.

It is an aim of certain embodiments of the invention to provide a method of prompt and sensitive characterization of protein or drug binding and function within the AP regulatory TMC and related complexes in real time.

It is an aim of certain embodiments of the invention to provide a method for identifying personalized medication for a subject expressing genetic complement system protein variants.

It is an aim of certain embodiments of the invention to provide a method for characterizing the impact of de novo CD46 (MCP), CD35 (CR1), CFH (which translates to FH and FHL-1), CFI and C3 (C3b) mutations.

### SUMMARY

In a first aspect of the present invention, there is provided a surface bound protein complex comprising:
a. a Complement C3b protein or variant thereof, or Complement C4b protein or a variant thereof,
b. a mutated Complement Factor I protein or variant thereof, and
c. a Complement Factor I co-factor protein,

wherein the Complement Factor I protein or variant thereof comprises at least one mutation that results in an inactivation or reduction of Complement Factor I protease activity.

In certain embodiments, the protein complex comprises C3b, and further wherein the Complement Factor I cofactor protein is selected from:
a. a Complement Factor H protein or variant thereof; and/or
b. Factor-H-like protein 1 or variant thereof; and/or
c. soluble membrane cofactor protein (sMCP) or variant thereof, and/or
d. soluble complement receptor 1 (sCR1) or variant thereof.

In certain embodiments, the protein complex comprises C4b, and further wherein the Complement Factor I cofactor protein is selected from:
a. soluble complement receptor 1 (sCR1) or variant thereof, and/or
b. C4b binding protein (C4 bp) or variant thereof, and/or
c. soluble membrane cofactor protein (sMCP) or variant thereof.

In certain embodiments, the mutation causing inactivation or reduction of Complement Factor I protease activity is at amino acid position 380, and/or amino acid position 429, and/or amino acid position 525.

In certain embodiments, the Complement Factor I protein comprises an S525A mutation and/or H380R mutation.

In certain embodiments, the mutation that results in inactivation or reduction of Complement Factor I protease activity is at amino acid position 380, and/or amino acid position 429, and/or amino acid position 525 according to the amino acid sequence as set forth in SEQ ID NO: 3. In certain embodiments, the complex is bound to the surface of a surface plasmon resonance sensor chip.

In certain embodiments, the sensor chip comprises a carboxymethyl group.

In certain embodiments, the complex is surface bound via an amine coupling, or a thiol coupling, or a coupling via a thioester.

In certain embodiments, the Complement Factor I or variant thereof is a mammalian Complement Factor I protein.

In certain embodiments, the mammalian Complement Factor I or variant thereof is a human Complement Factor I.

In certain embodiments, the human Complement Factor I protein comprises a first amino acid sequence selected from an amino acid sequence as set forth in SEQ ID NO: 4 (heavy chain) or an amino acid sequence having at least 85% sequence identity to the amino acid sequence as set forth in SEQ ID No: 4, and a second amino acid sequence as set forth in SEQ ID NO: 8 (light chain) or SEQ ID NO: 17 or an amino acid sequence having at least 85%, e.g. at least 90% sequence identity to the amino acid sequence set forth in SEQ ID NO: 8 or SEQ ID NO: 17, wherein the first and second amino acid sequences are linked via a disulphide bond.

In certain embodiments, the Complement Factor H or variant thereof is a mammalian Complement Factor H protein.

In certain embodiments, the mammalian Complement Factor H is a human Complement Factor H protein.

In certain embodiments, the human Complement Factor H protein comprises an amino acid sequence selected from an amino acid sequence as set forth in SEQ. ID. No. 2 or an amino acid sequence having at least 85%, e.g. 90% sequence identity to the amino acid sequence as set forth in SEQ. ID. No. 2.

In certain embodiments, the human Complement Factor H protein comprises an amino acid sequence selected from an amino acid sequence as set forth in SEQ ID NO: 1 or an amino acid sequence having at least 85%, e.g. 90% sequence identity to the amino acid sequence as set forth in SEQ ID NO: 1.

In certain embodiments, the C3b or variant thereof is a mammalian C3b.

In certain embodiments, the mammalian C3b is a human C3b protein.

In certain embodiments, the human C3b protein comprises an amino acid sequence selected from an amino acid sequence as set forth in SEQ ID NO: 9 or an amino acid sequence having at least 85%, e.g. 90% sequence identity to the amino acid sequence as set forth in SEQ. ID. No. 9.

In certain embodiments, the Factor-H-like protein 1 or variant thereof is a mammalian Factor-H-like protein 1.

In certain embodiments, the mammalian Factor-H-like protein 1 is a human Factor-H-like protein 1.

In certain embodiments, the human Factor-H-like protein 1 comprises an amino acid sequence selected from an amino acid sequence as set forth in SEQ ID NO: 15 or an amino acid sequence having at least 85% sequence identity to the amino acid sequence as set forth in SEQ ID NO: 15.

In certain embodiments, the soluble membrane cofactor protein or variant thereof is a mammalian soluble membrane cofactor protein.

In certain embodiments, the mammalian soluble membrane cofactor protein is a human soluble membrane cofactor protein.

In certain embodiments, the human soluble membrane cofactor protein comprises an amino acid sequence selected from an amino acid sequence as set forth in SEQ ID NO: 13 or an amino acid sequence having at least 85%, e.g. 90% sequence identity to the amino acid sequence as set forth in SEQ ID NO: 13.

In certain embodiments, the soluble complement receptor 1 protein or variant thereof is a mammalian soluble complement receptor 1 protein.

In certain embodiments, the mammalian soluble complement receptor 1 protein is a human soluble complement receptor 1 protein.

In certain embodiments, the human soluble complement receptor 1 protein comprises an amino acid sequence selected from an amino acid sequence as set forth in SEQ ID NO: 11 or an amino acid sequence having at least 85%, e.g. 90% sequence identity to the amino acid sequence as set forth in SEQ ID NO: 11.

In a further aspect of the present invention, there is provided a method for determining the presence, absence or characteristics of a binding event associated with a protein complex, the protein complex comprising a mutant Complement Factor I protein or variant thereof, wherein the protein complex further comprises at least one of the following proteins;
a. a Complement C3b protein or variant thereof, or Complement C4b protein or a variant thereof, and
b. a Complement factor I cofactor protein,

wherein the Complement Factor I protein or variant thereof comprises at least one mutation that reduces or inactivates Complement Factor I protein protease activity of the Complement Factor I protein, wherein the method comprises:
i) forming said protein complex; and
ii) detecting the presence, absence and/or characteristics of a signal produced by a binding event associated with the formation of said protein complex, wherein said signal is produced by interaction of said Complement Factor I with said Complement C3b or variant thereof, and/or said Complement Factor I cofactor protein.

In certain embodiments, the method comprises forming a protein complex wherein, the protein complex comprises C3b, and further wherein the complement factor I cofactor protein is Complement Factor H protein or a variant thereof, or Factor-H-like protein 1 or a variant thereof, or soluble membrane cofactor protein (sMCP) or a variant thereof, or soluble complement receptor 1 (sCR1) or a variant thereof.

In certain embodiments, the method comprises forming a protein complex, wherein the protein complex comprises C4b, and further wherein the complement factor I cofactor is soluble complement receptor 1 (sCR1) or a variant thereof, or soluble membrane cofactor protein (sMCP) or a variant thereof, or C4b binding protein (C4 bp) or a variant thereof.

In certain embodiments, the method further comprises first binding a component of said protein complex to a surface, wherein optionally the surface is a surface plasmon resonance sensor chip.

In certain embodiments, the method comprises binding a component of said protein complex to the surface via amine coupling, or thiol coupling, or a coupling via a thioester.

In certain embodiments, the method comprises binding the Complement C3b protein or variant thereof to the surface.

In certain embodiments, the method comprises detecting the presence or absence of a signal generated by a binding event associated with the formation of said protein complex using a sensor configured to detect said signal.

In certain embodiments, the method comprises detecting the presence, absence and/or characteristics of the binding event with a sensor configured to detect surface plasmon resonance.

In certain embodiments, the method comprises forming a protein complex wherein the mutation that reduces or inactivates Complement Factor I protein protease activity of the Complement Factor I protein is at amino acid position 380, and/or amino acid position 429, and/or amino acid position 525.

In certain embodiments, the Complement Factor I protein comprises an S525A mutation and/or H380R mutation.

In certain embodiments, the amino acid mutation at amino acid position 380, and/or amino acid position 429, and/or amino acid position 525 are positioned according to the amino acid sequence as set forth in SEQ. ID. No: 3.

In certain embodiments, the surface bound complex further comprises any one or more of the features recited herein above.

In certain embodiments, the method further comprises determining the binding affinity of the interaction of said Complement Factor I with said Complement C3b or variant thereof, and/or said Complement Factor I cofactor protein.

In certain embodiments, the method further comprises identifying a biomarker in a subject. In a further aspect of the present invention, there is provided a method for determining the presence, absence, or characteristics of a ligand binding event associated with a protein complex comprising a mutant Complement Factor I protein or variant thereof, wherein the protein complex further comprises at least one of the following proteins;
a. a Complement C3b protein or variant thereof, or Complement C4b protein or a variant thereof, and
b. a Complement Factor I cofactor protein,

wherein the Complement factor I or variant thereof comprises at least one mutation that reduces or inactivates Complement Factor I protease activity of the Complement Factor I protein, and wherein the method comprises;
i) forming said protein complex, and
ii) contacting said protein complex with a candidate ligand molecule; and
iii) detecting the presence, absence, and/or characteristics of a signal generated by an interaction of said ligand molecule with said protein complex.

In certain embodiments, the method comprises forming a protein complex wherein the Complement Factor I cofactor protein is selected from a Complement Factor H protein or variant thereof, a Factor-H-like protein 1 or variant thereof, a C4b binding protein or a variant thereof, a soluble membrane cofactor protein (sMCP) or variant thereof, and a soluble complement receptor 1 (sCR1) or variant thereof.

In certain embodiments, the method comprises first binding a component of the protein complex to a surface, wherein optionally the surface is surface plasmon resonance sensor chip.

In certain embodiments, the method comprises binding a component of the protein complex to said surface via amine coupling, or thiol coupling, or a coupling via a thioester.

In certain embodiments, the method comprises binding the Complement C3b protein or variant thereof to the surface.

In certain embodiments, the method comprises detection of the presence, absence and/or characteristic of a signal generated by an interaction of said candidate ligand molecule with said protein complex using a sensor configured to detect said signal.

In certain embodiments, the method comprises using a sensor configured to detect surface plasmon resonance.

In certain embodiments, the complex further comprises any one or more of the features herein above or a permissible combination thereof.

In certain embodiments, the method further comprises determining a binding affinity of a ligand molecule with a protein complex.

In certain embodiments, the method further comprises identifying a biomarker in a subject.

In certain embodiments, the invention provides a surface comprising a complex according to any one of the above.

Certain aspects of the invention provide a surface bound protein complex comprising:
a. a Complement C3b protein or variant thereof, or a Complement C4b protein or variant thereof,
b. a mutated Complement Factor I protein or variant thereof, and
c. a Complement Factor I co-factor protein,

wherein the Complement Factor I protein or variant thereof comprises at least one mutation within its active site that results in an inactivation or reduction of Complement Factor I protease activity.

Certain aspects of the present invention provide a method for determining the presence, absence or characteristics of a binding event associated with a protein complex, the protein complex comprising a mutant Complement Factor I protein or variant thereof, wherein the protein complex further comprises at least one of the following proteins;
a. a Complement C3b protein or variant thereof, or a Complement C4b protein or variant thereof, and/or
b. a Complement factor I cofactor protein,

wherein the Complement Factor I protein or variant thereof comprises at least one mutation in its active site that reduces or inactivates Complement Factor I protein protease activity of the Complement Factor I protein, wherein the method comprises:
i) forming said protein complex; and
ii) detecting the presence, absence and/or characteristics of a signal produced by a binding event associated with the formation of said protein complex, wherein said signal is produced by interaction of said Complement Factor I with said Complement C3b or variant thereof, or a Complement C4b protein or variant thereof and/or said Complement Factor I cofactor protein.

Certain aspects of the present invention provide a method for determining the presence, absence, or characteristics of a ligand binding event associated with a protein complex comprising a mutant Complement Factor I protein or variant thereof, wherein the protein complex further comprises at least one of the following proteins;
a. a Complement C3b protein or variant thereof, or a Complement C4b protein or variant thereof, and/or
b. a Complement Factor I cofactor protein,

wherein the Complement factor I or variant thereof comprises at least one mutation in its active site that reduces or inactivates Complement Factor I protease activity of the Complement Factor I protein, and wherein the method comprises;
i) forming said protein complex, and
ii) contacting said protein complex with a candidate ligand molecule; and
iii) detecting the presence, absence, and/or characteristics of a signal generated by an interaction of said ligand molecule with said protein complex.

The following abbreviations may be used herein:

### List of Abbreviations

AD— Alzheimer disease
aHUS— atypical haemolytic uremic syndrome (aHUS)
AMD— Age-related macular degeneration
AP— Alternative pathway of the complement System
AUC— analytical ultracentrifugation
BLI— Biolayer interferometry
CCP— complement-control-protein domain
CD— circular dichroism
CD35— cluster of differentiation 35
CD46— cluster of differentiation 46
CFB— Complement Factor B gene
CFD— Complement Factor D gene
CFH— Complement Factor H gene
CFI— Complement Factor I gene
CHO— Chinese hamster ovary
CR1— complement receptor 1
EDC— N-(3-dimethylaminopropyl)-N′-ethylcarbodimide hydrochloride
ELISA— Enzyme linked immunosorbent assay
EMSA— electrophoretic mobility shift assay
FB— Complement Factor B protein
FD— Complement Factor D protein
FH— Complement Factor H protein
FH1-4— recombinant FH CCP domains 1-4
FHL-1— Factor-H like protein 1
FI— Complement Factor I protein
FLFH— Full length Complement Factor H
FRET— Forster Resonance Energy Transfer
GWAS— Genome wide association studies
HBS— HEPES buffered saline
HC— Heavy chain
ICD— Induced circular dichroism
ISH— in situ hybridization
ITC— Isothermal titration calorimetry
LC— Light chain
MA— methylamine
MCP— Membrane cofactor protein
sMCP— Soluble (recombinant) membrane cofactor protein
MST— Microscale thermophoresis
NHS— N-hydroxysuccinimide
NRCTC— National Renal Complement Therapeutics Centre
PBS— phosphate-buffered saline
PNH— paroxysmal nocturnal hemoglobinuria
RA— Radio-immunoassay
RT— room temperature
RU— resonance units
SCR— short consensus repeats
sCR1— soluble complement receptor 1
SI— Systeme International d'Unités
SPR— Surface Plasmin Resonance
TMC— Complement trimolecular complex
WT— wild-type

### DETAILED DESCRIPTION OF CERTAIN EMBODIMENTS OF THE INVENTION

Embodiments of the invention will now be described by way of example only, and with reference to the accompanying Figures in which:

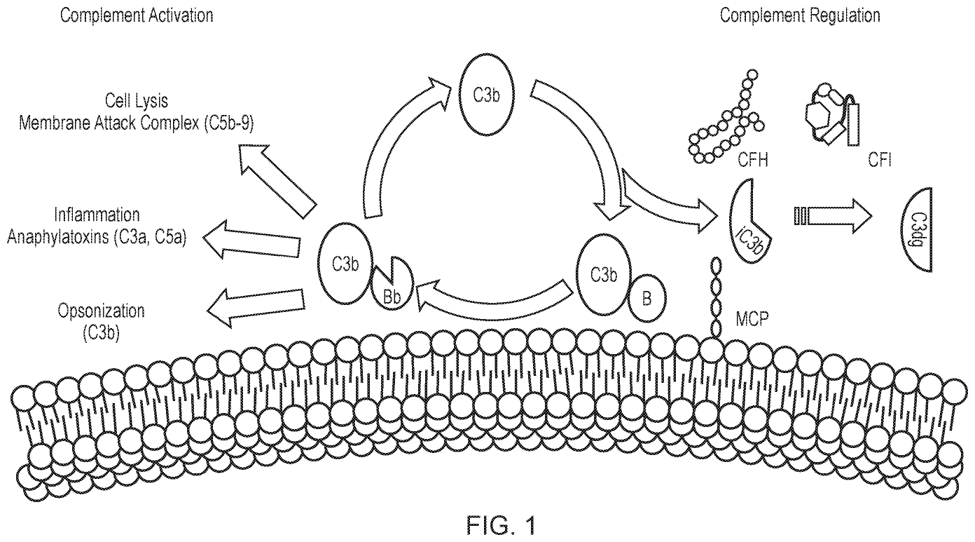

FIG. 1 shows a simplified schematic representation of the Complement Pathway.

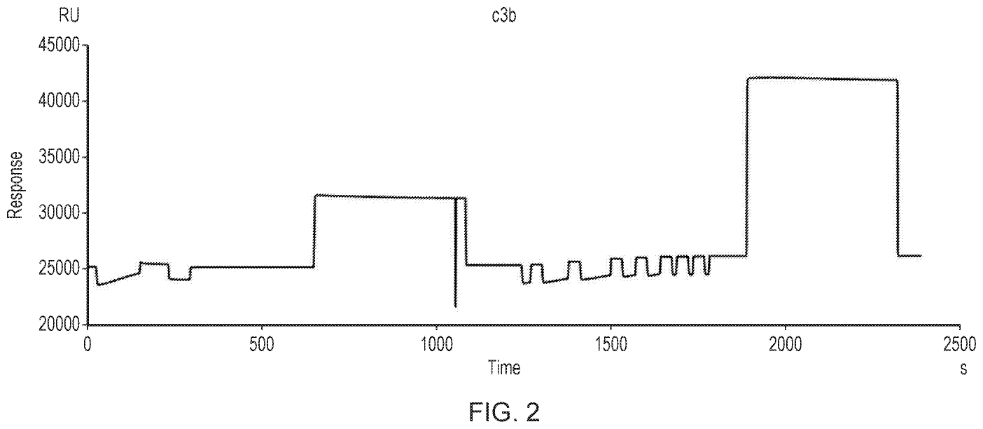

FIG. 2 shows a sensorgram produced in the BIAcore S200 BIAevaluation software displaying C3b being immobilised to a CM5 chip by amine coupling post-activation by NHS and EDC, the plot gives RU (x-axis) as a function of time (y-axis). It is possible to see here incremental increases, in response to each flow period, in bound C3b on the surface of the chip, with the RU starting at ˜25200RU and finishing at ˜26000RU indicating that 800RU of C3b was immobilised.

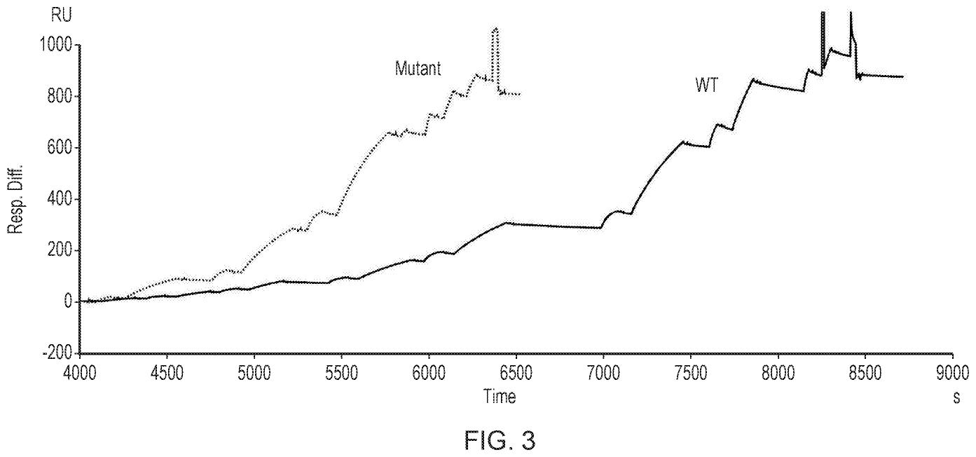

FIG. 3 shows a sensorgram produced in the BIAcore S200 BIAevaluation software displaying C3b (WT & L1109V mutant) being immobilised to a CM5 chip by thiol coupling following immobilisation of a small nidus of C3 Methylamine using NHS/EDC, the plot gives RU (x-axis) after subtracting the response of the blank flow cell as a function of time (y-axis). It is possible to see here incremental increases, in response to each flow period, in bound C3 convertase and C3b on the surface of the chip, with the amount of C3b binding to the surface increasing per unit of time after each injection of Complement Factor B (FB) and Complement Factor D (FD) as the chip surface becomes more saturated with C3 convertase. RU starting at ˜0RU and finishing at ˜800-900RU indicated that C3b was immobilised.

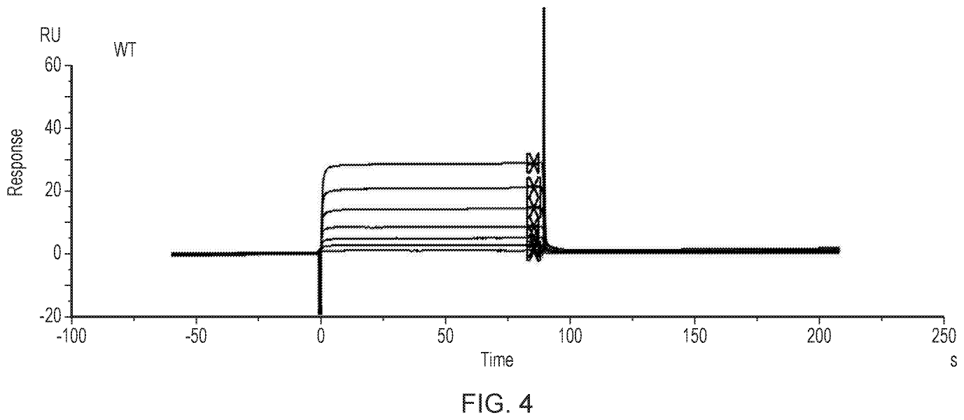

FIG. 4 shows a sensorgram produced using the BIAcore S200 BIAevaluation software showing the steady state binding of FH1-4 (WT) to C3b on the CM5 chip after subtracting the blank flow cell from the C3b-immobilised flow cell to give relative RU against time (s) during the flow period. Decreasing concentrations (starting from the highest RU) gave decreasing response sizes, each concentration is represented by an individual line.

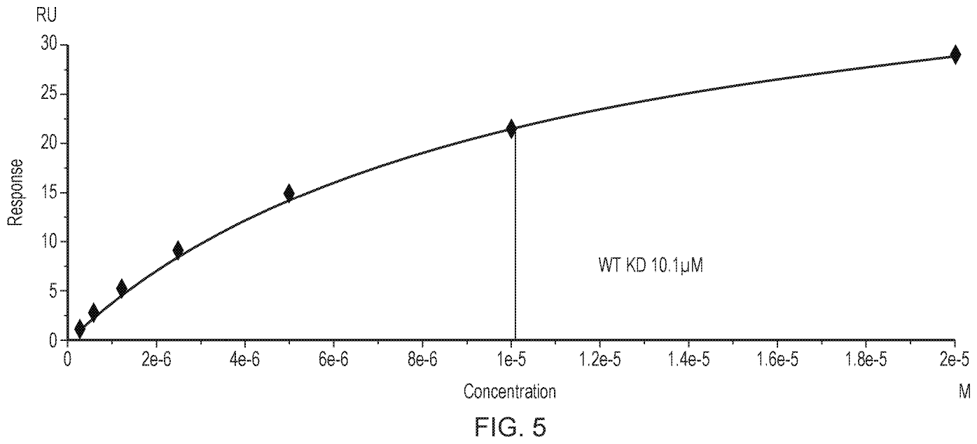

FIG. 5 shows an affinity calculation curve produced using the BIAcore S200 BIAevaluation software showing the relative responses in RU on the Y axis, given after injections of varying concentrations (M) of WT FH CCP1-4, as displayed on the X axis. The line drawn shows the concentration at which 50% of RUmax was reached (the K_{D}).

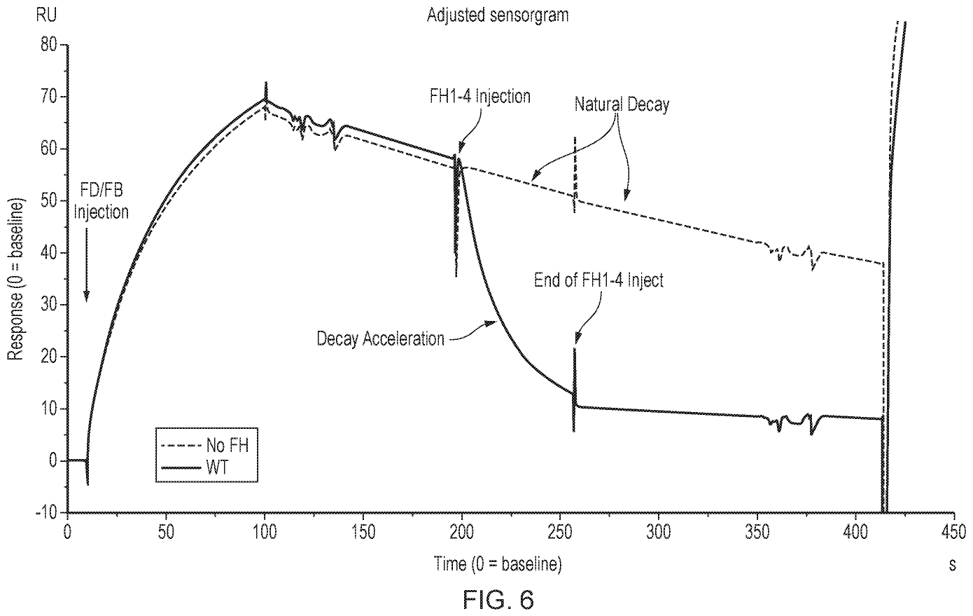

FIG. 6 shows a sensorgram produced using the BIAcore S200 BIAevaluation software, showing normal decay accelerating activity induced by WT FH1-4 after injection of FD and FB combined to build the C3 convertase, followed by injection of 250 nM WT FH1-4 compared to injection of no buffer only, on a C3b-coupled CM5 chip (RU given on Y-axis is normalised to a no C3b control chip).

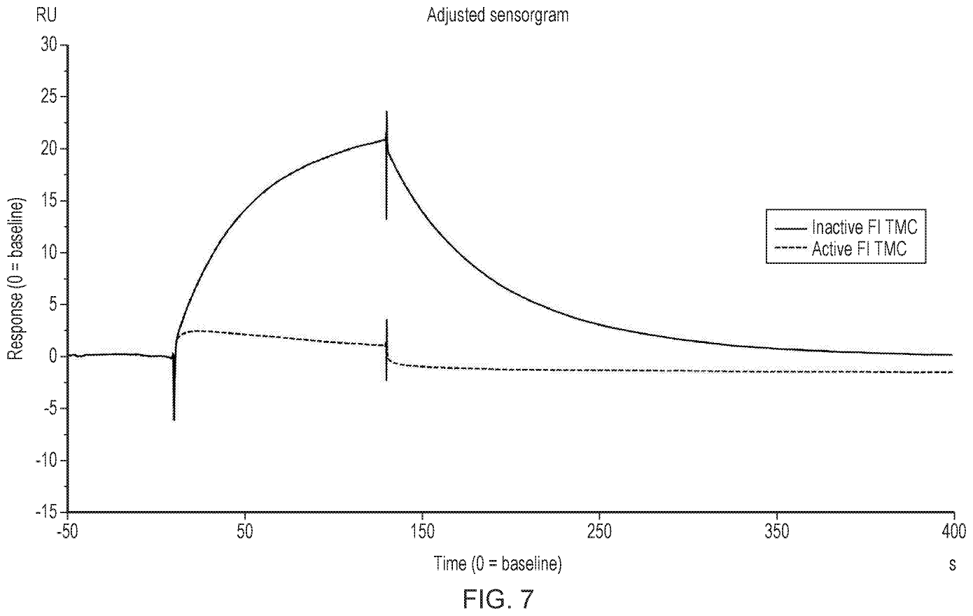

FIG. 7 shows an adjusted sensorgram displaying the binding response in RU after injection of inactive (S525A) FI and FH1-4 onto a C3b amine-coupled CM5 chip (solid line), in comparison to the injection of active FI and FH (dotted line), both at 69 nM FH1-4 and 118 nM FI. A substantial RU increase can be seen during injection of inactive (S525A) FI, caused by synergistic binding of the three proteins in the TMC. However, with active FI at RT, C3b on the chip is cleaved into iC3b which causes C3f to dissociate and FH to lose affinity to the chip surfaces increasingly, as more C3b is converted into iC3b (as shown by the downward trend after initial injection). FI and FH1-4 dissociate immediately after injection stopping, which is highlighted by the appearance of steady state (fast on/off) binding.

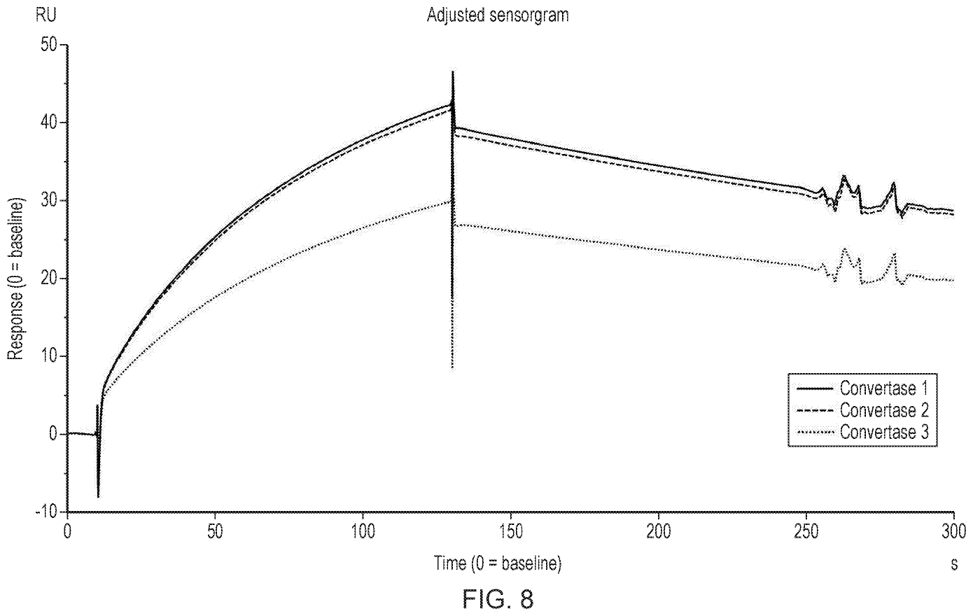

FIG. 8 shows a sensorgram produced by injections of 500 nM FB and 60 nM FD which were made onto an C3b amine-coupled CM5 chip using the BIAcore S200 to form convertase 1; before TMC building assays were performed, convertase 2; after all TMC building with inactive (S525A) FI was completed and convertase 3; after cleavage of the chip surface C3b by WT (active) FI. As shown by the above adjusted sensorgram, the convertase built after cleavage of the chip was greatly reduced, indicative of loss of active surface due to C3b cleavage to iC3b.

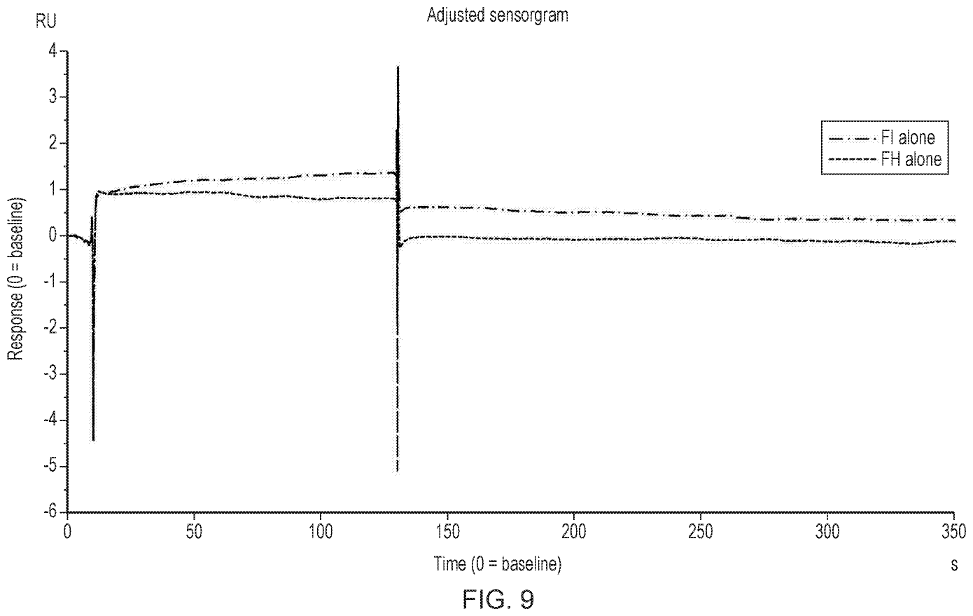

FIG. 9 shows sensorgrams of inactive FI ((S525A)) and FH1-4-only injections, which were made at 118 nM onto a C3b amine-coupled CM5 chip on the BIAcore S200, these were RU adjusted by initial injection over a blank Fc to give this sensorgram showing the small relative binding of the individual proteins (FI is shown by the short/long dashed line and FH1-4 is shown by the short-dashed line).

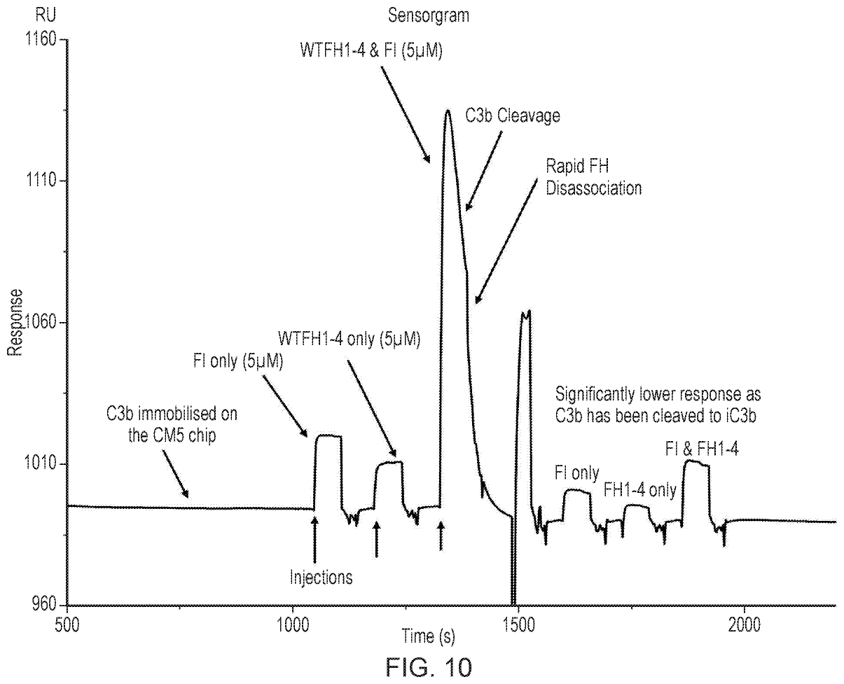

FIG. 10 shows a sensorgram produced using the BIAevaluation software S200 (GE) showing a manual TMC injection run as revealed by SPR. C3b (˜800RU) was immobilised to the CM5 chip before injection, at 4° C., of 5 μM FI and FH1-4 separately, before 5 μM of both simultaneously, all in PBST. The first TMC build, which resulted in binding of all 3 proteins causing cleavage, is followed by a wash before the same components were injected a second time with minimal response. Time (x-axis, seconds) is plotted versus response (RU) after normalisation by injection over a blank flow cell.

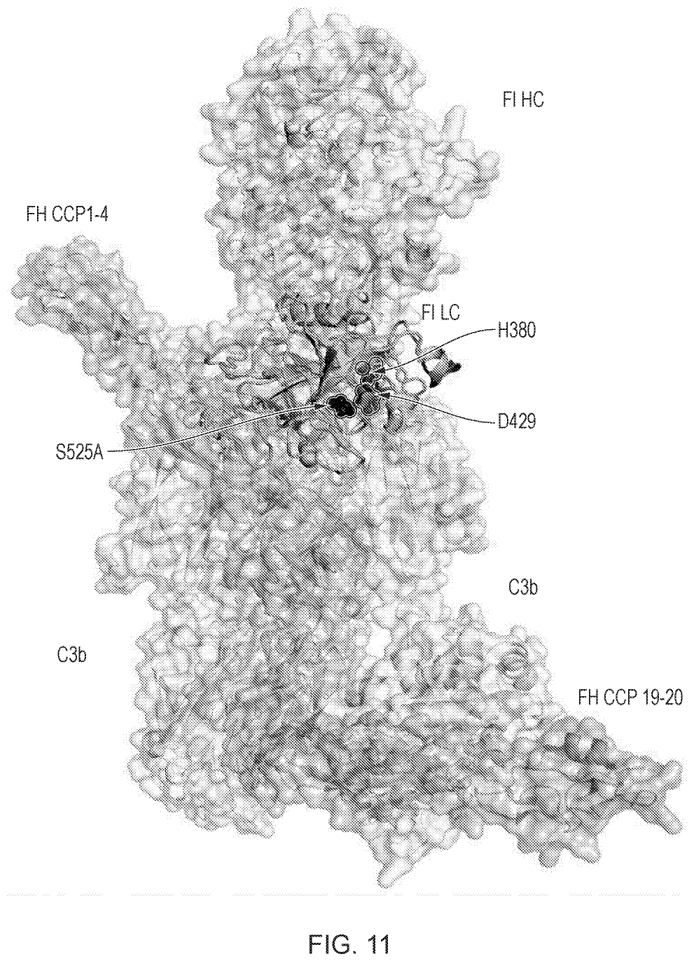

FIG. 11 shows a protein complex comprising C3b, FH1-4, and FI. FI is represented by ribbons (LC) and a cartoon (HC). C3b is in pale and FH CCPs 1-4 and 19-20 are displayed, with CCP1 of FH situated at the top left of the diagram. The catalytic triad of FI is represented by spheres (H380 and D429, S525).

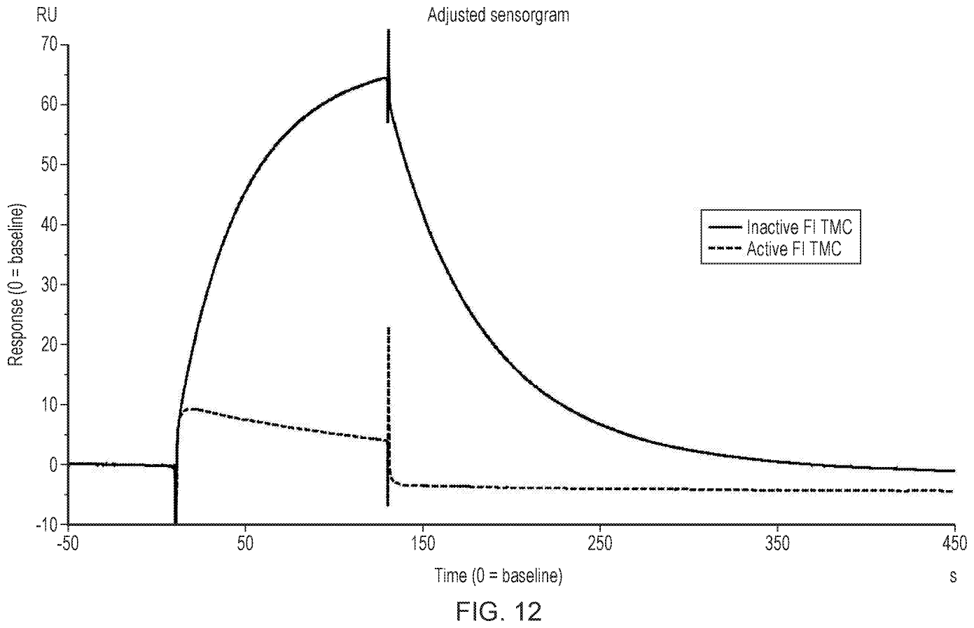

FIG. 12 shows a sensorgram produced in the BIAevaluation software S200 (GE) after injection of 118 nM of WT FI and WT FH1-4 (dotted line), or inactive (S525A) FI and WT FH1-4 (solid line) onto a C3b-couple chip (˜1000RU, thiol coupled) in PBST. Time (x-axis, seconds) is plotted versus response (RU) after normalisation by injection over a blank flow cell.

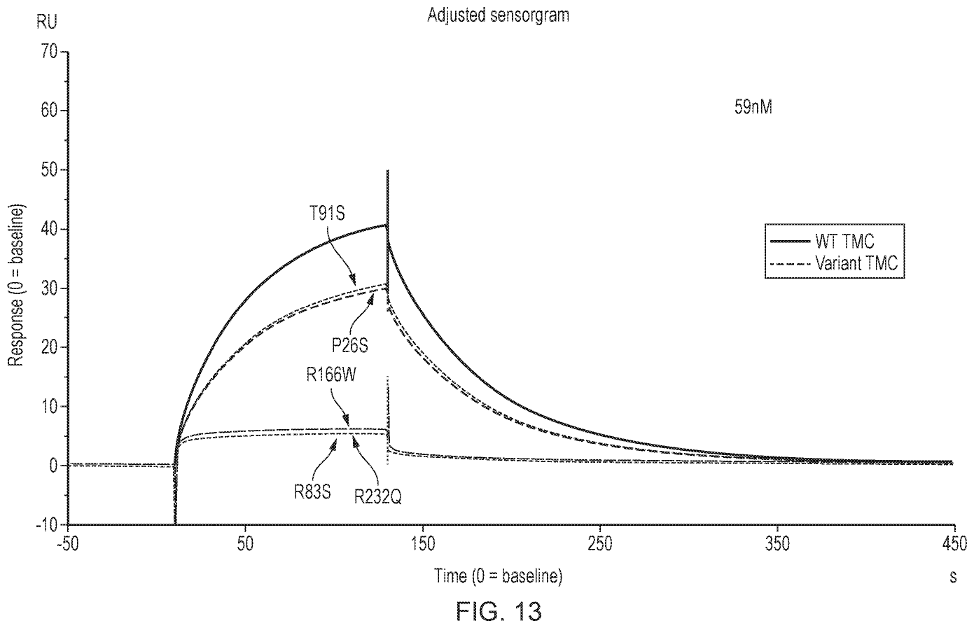

FIG. 13 shows a sensorgram produced in the BIAevaluation software S200 (GE) after injection of 59 nM inactive (S525A) FI and each FH1-4 variant (dotted lines, P26S, R83S, T91, R166W, R232Q), or inactive (S525A) FI and WT FH1-4 (solid line) onto a C3b-coupled chip (˜1000RU, thiol coupled). Time (x-axis, seconds) is plotted versus response (RU) after normalisation by injection over a blank flow cell.

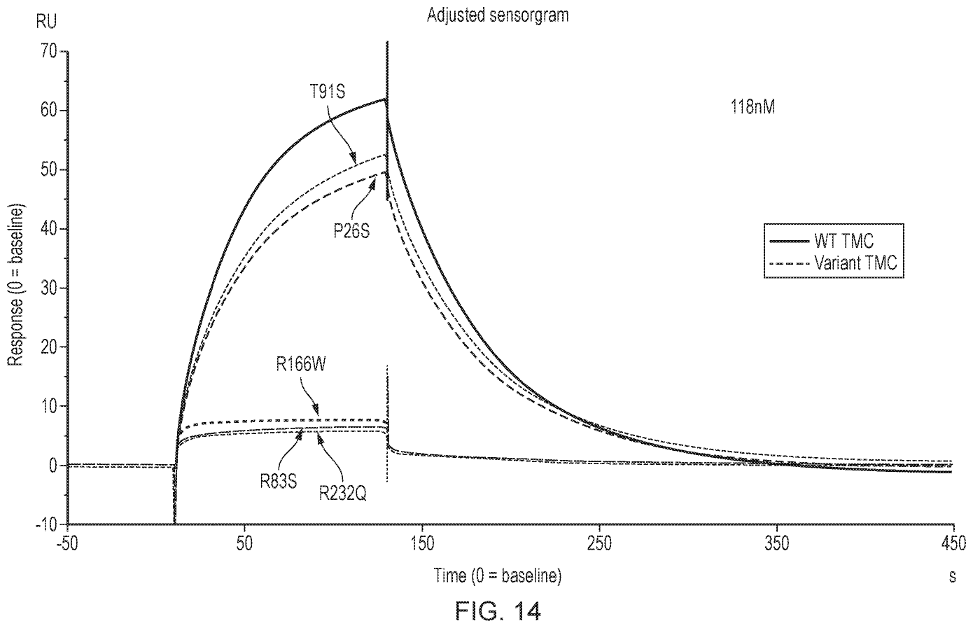

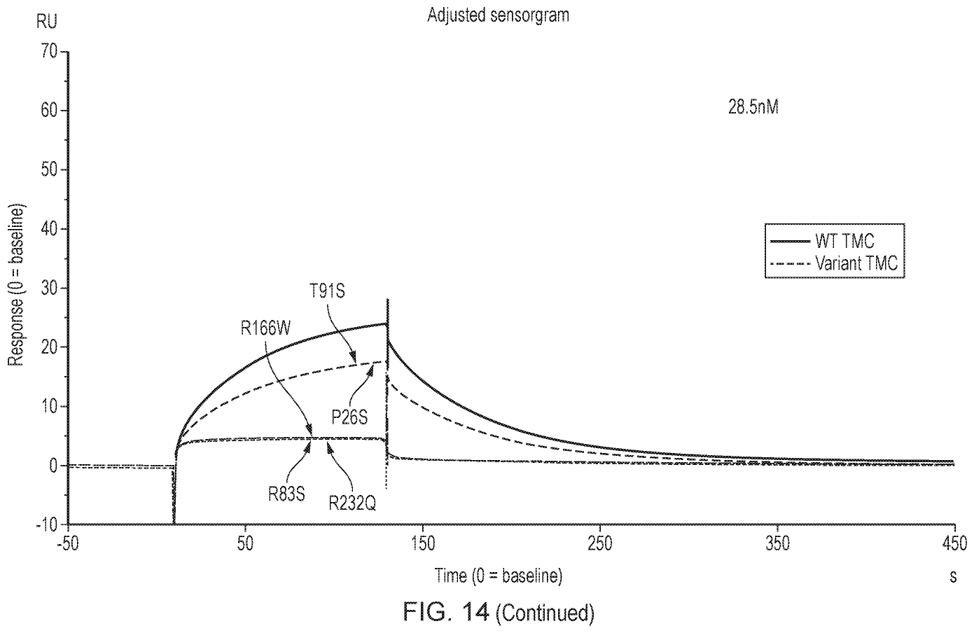

FIG. 14 shows a sensorgram produced in the BIAevaluation software S200 (GE) after injection of 118 nM (left) and 28.5 nM (right) of inactive (S525A) FI and each FH1-4 variant (dotted lines), or inactive (S525A) FI and WT FH1-4 (solid line) onto a C3b-coupled chip (˜1000RU, thiol coupled). Time (x-axis, seconds) is plotted versus response (RU) after normalisation by injection over a blank flow cell.

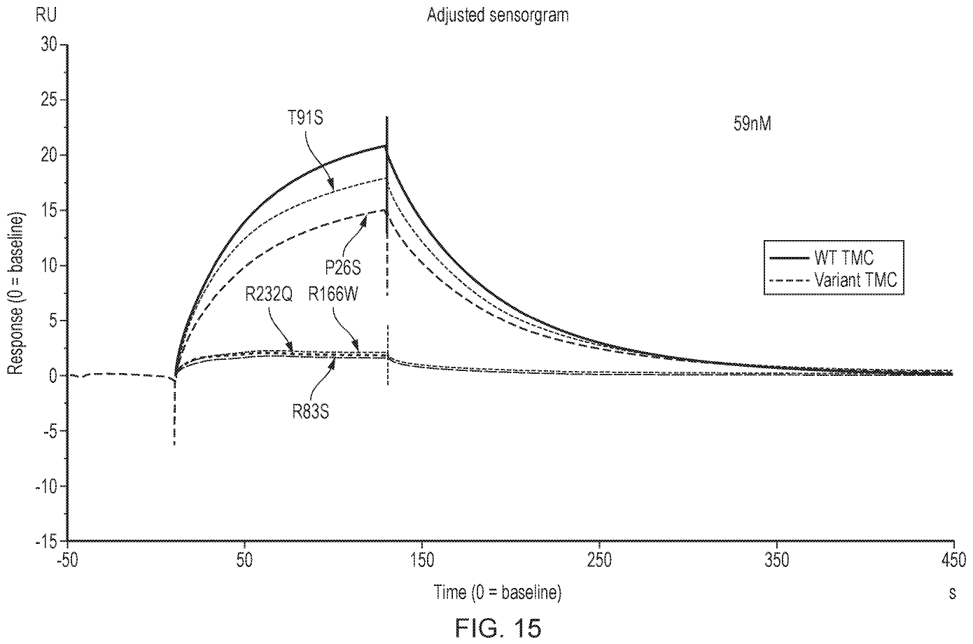

FIG. 15 shows a sensorgram produced in the BIAevaluation software S200 (GE) after injection of 59 nM inactive FI (S525A) and each variant in FH1-4 (dotted lines, labelled P26S, R83S, T91S, R166W, R232Q), or inactive (S525A) FI and WT FH1-4 (solid line) onto a C3b-coupled chip (˜1000RU, amine coupled). Time (x-axis, seconds) is plotted versus response (RU) after normalisation by injection over a blank flow cell.

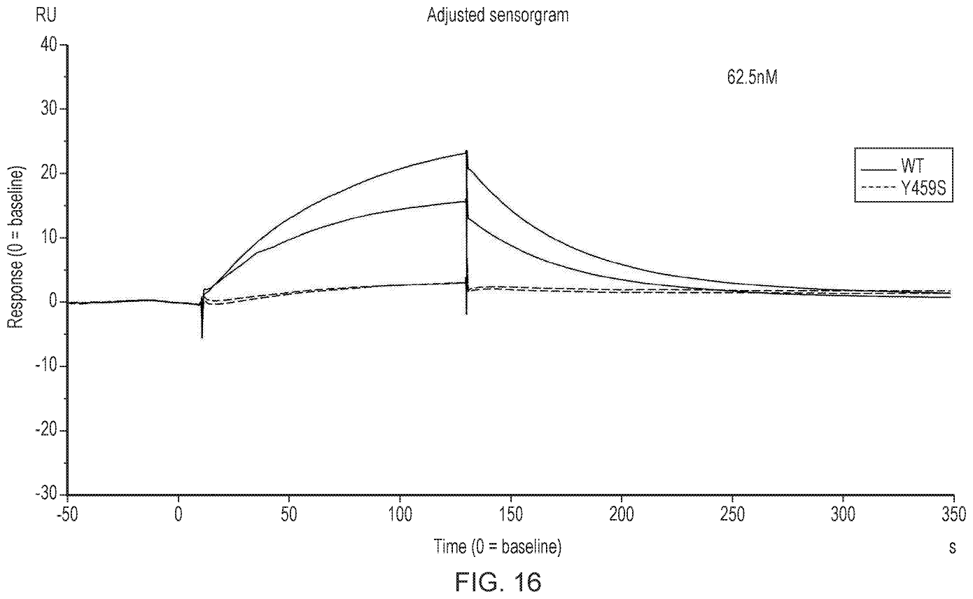

FIG. 16 shows a sensorgram produced using the BIAevaluation software S200 (GE) after injection of 62.5 nM of S525A (inactive) FI and WT FH1-4 (solid line), or Y459S inactive (S525A) FI and WT FH1-4 (dotted line) onto a C3b-couple chip (˜1000RU, amine coupled) in PBST. Time (x-axis, seconds) is plotted versus response (RU) after normalization by injection over a blank flow cell.

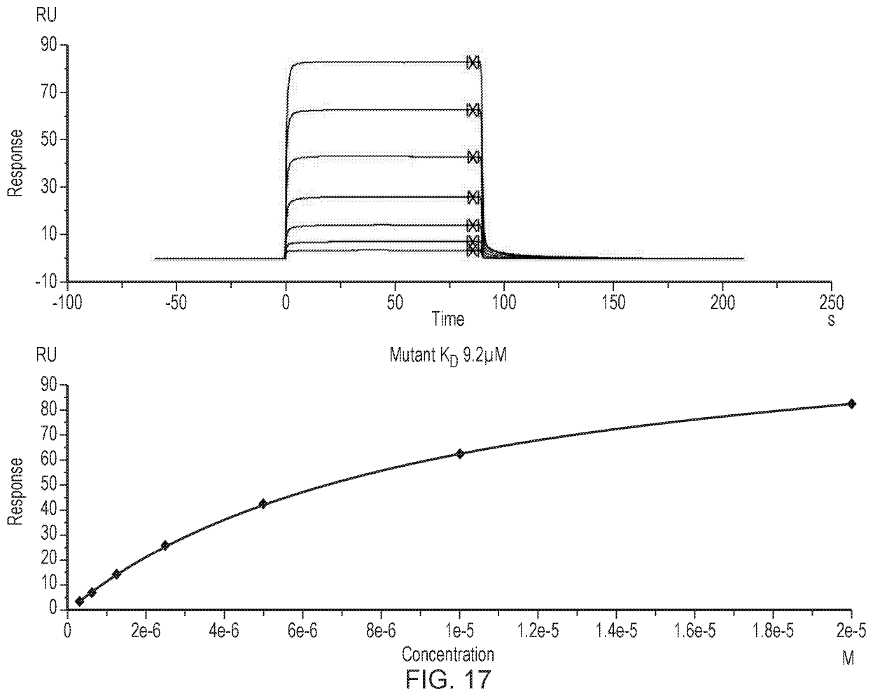

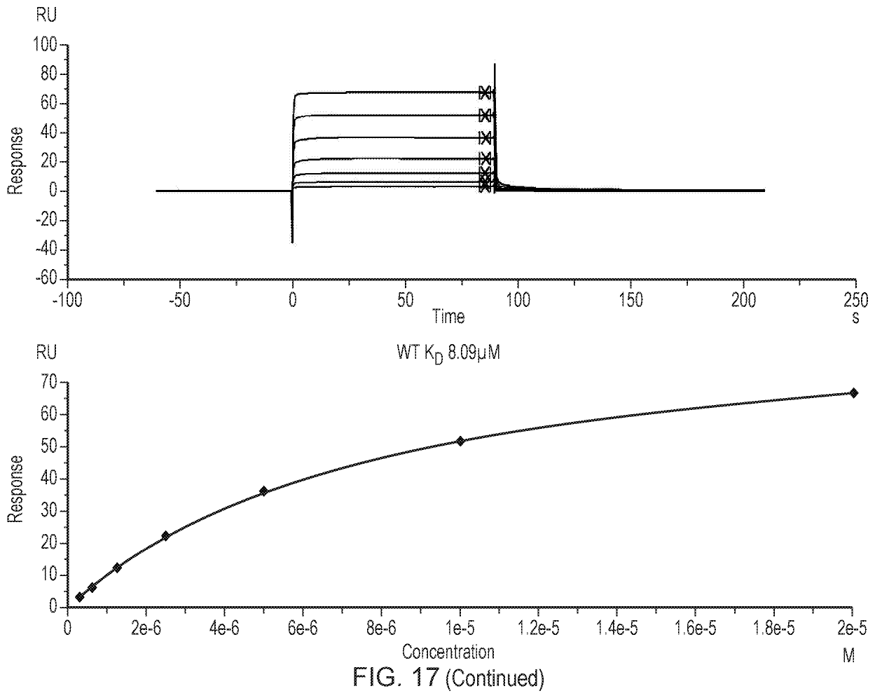

FIG. 17 shows sensorgrams produced using BIAevaluation S200 (GE) software showing response (RU) as a function of time after injection of varying concentrations of FH1-4 across a WT (top left) and L1109V Mutant (top right) C3b thiol-coupled (899 and 898 RU, respectively, on separate flow cells) CM5 chip on the BIAcore S200, with the highest FH1-4 concentration giving the largest response. Further sensorgrams with response (RU) as a function of each of the injection concentrations (in M) plotted for mutant and WT C3b are displayed at bottom left and bottom right, respectively. WT FH1-4 was injected at 20 μM then subsequently at concentrations resulting from a 1:2 serial dilution down to 0.156 μM in PBST. Concentration vs RU curves were used to calculate dissociation constants (K_{D}, in μM) at 50% maximal response as determined automatically by the BIAevaluation (GE) software affinity calculation programme, with the offset always set to constant (0), so the y intercept of the line of best fit always equals zero. Dissociation constants (K_{D}) are given under the graph showing each concentration vs RU curve labelled with the appropriate variant.

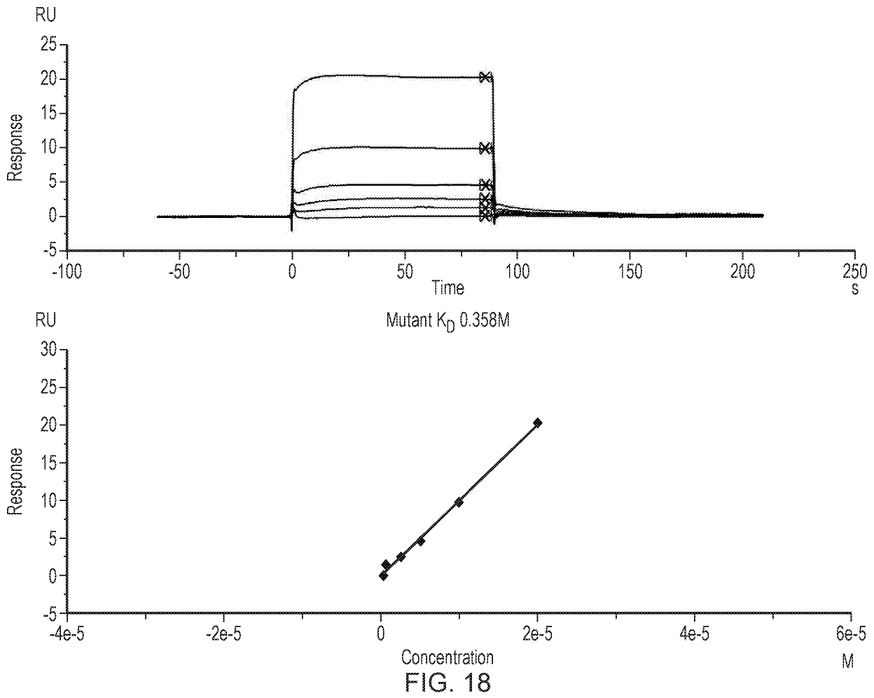

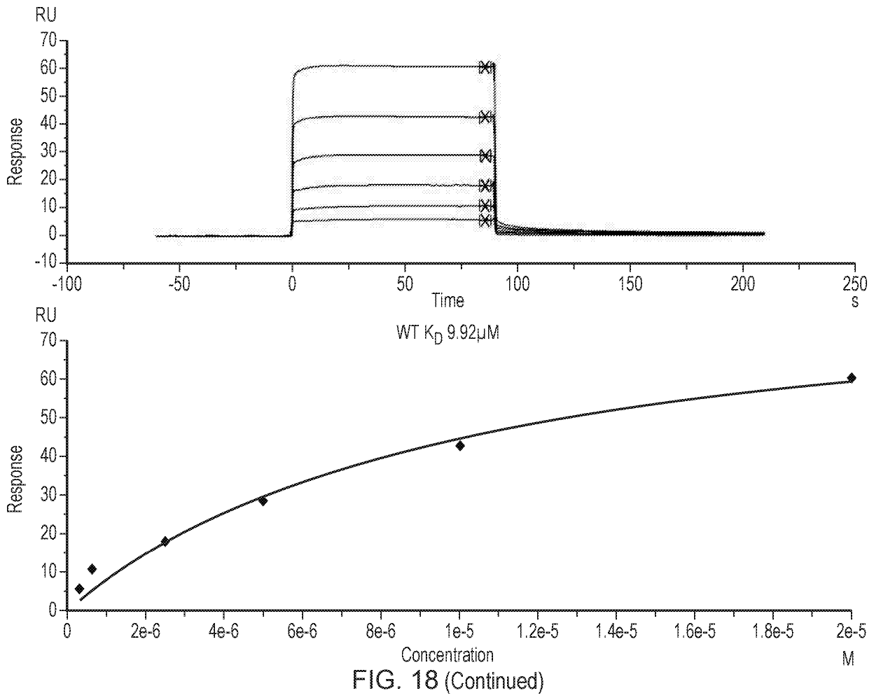

FIG. 18 shows a sensorgrams produced using BIAevaluation S200 (GE) software showing response (RU) as a function of time after injection of varying concentrations of FH19-20 across a WT (top left) and Mutant (top right) C3b thiol-coupled (899 and 898 RU, respectively, on separate flow cells) CM5 chip on the BIAcore S200, with the highest FH19-20 concentration giving the largest response. Further sensorgrams with response (RU) as a function of each of the injection concentrations (in M) plotted for mutant and WT C3b are displayed at bottom left and bottom right, respectively. WT FH CCP19-20 was injected at 20 μM then subsequently at concentrations resulting from a 1:2 serial dilution down to 0.156 μM in PBST. Concentration vs RU curves were used to calculate dissociation constants K_{D}, in μM) at 50% maximal response as determined automatically by the BIAevaluation S200 (GE) software affinity calculation programme, with the offset always set to constant (0), so the y intercept of the line of best fit always equals zero. Dissociation constants (K_{D}) are given under the graphs showing each concentration vs RU curve labelled with the appropriate variant.

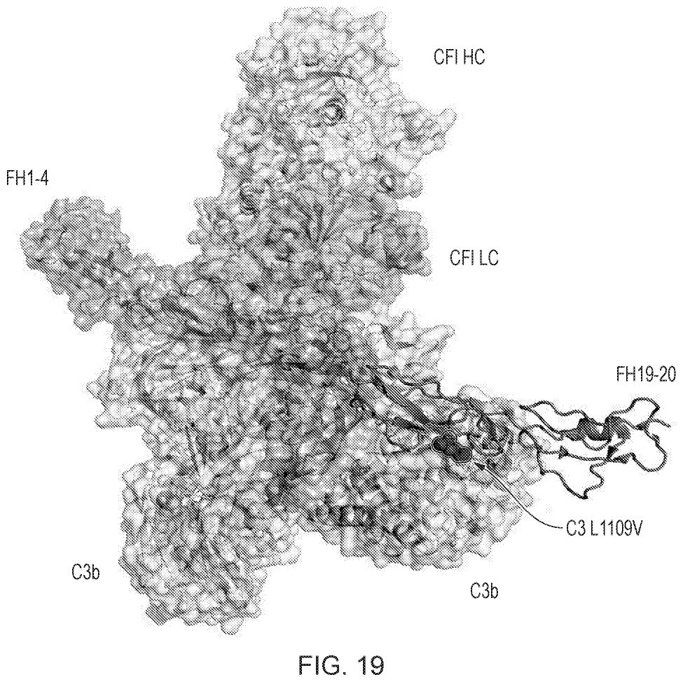

FIG. 19 shows the structural model of a rare C3 variant (spheres) that was identified in an aHUS patient. FH19-20 is displayed by the cartoon.

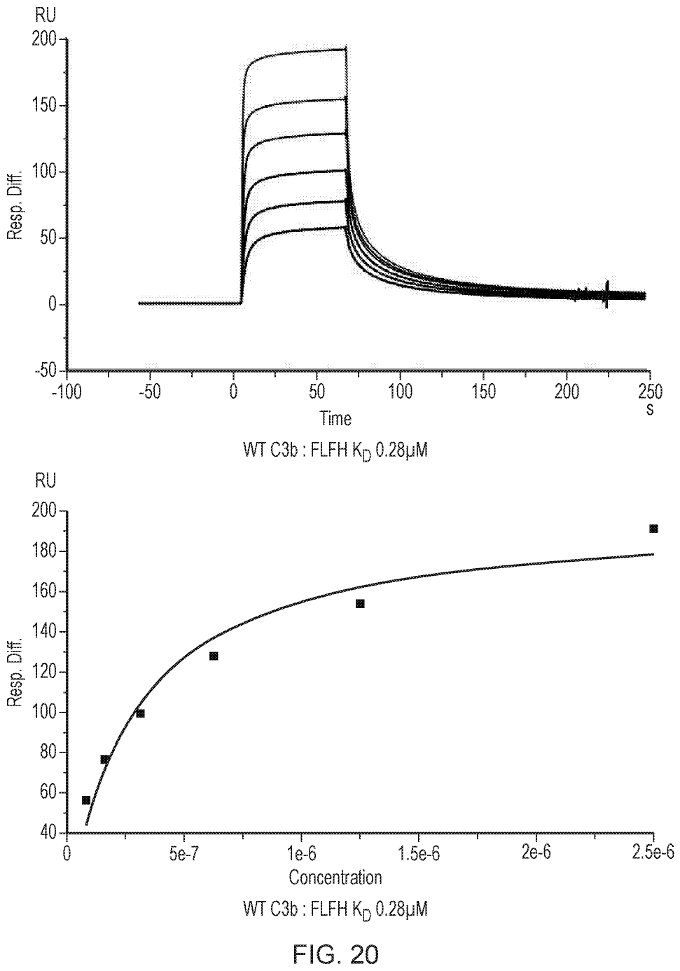

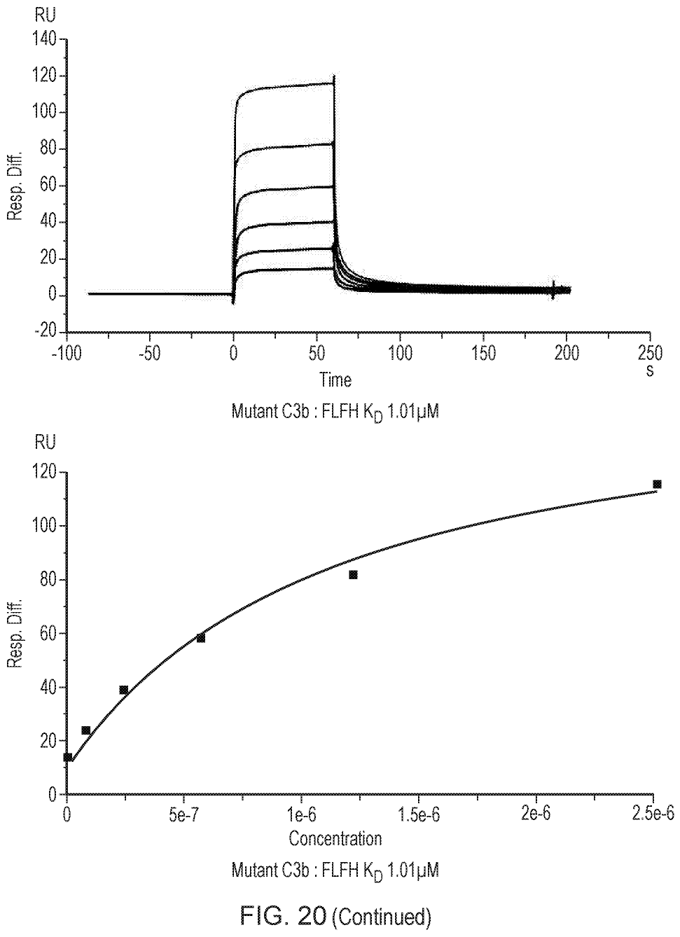

FIG. 20 shows a sensorgram produced using BIAevaluation (GE) software showing response (RU) as a function of time after injection of varying concentrations of full length FH (FLFH) across a WT (left) and Mutant (right) C3b thiol-coupled (899 and 898 RU, respectively, on separate flow cells) CM5 chip on the BIAcore S200, with the highest FLFH concentration giving the largest response. Further sensorgrams with response (RU) as a function of each of the injection concentrations (in M) plotted for WT and mutant C3b are displayed at top left and bottom left, respectively. WT FLFH was injected at 250 nM then subsequently at concentrations resulting from a 1:2 serial dilution down to 7.8 nM in PBST. Concentration vs RU curves were used to calculate dissociation constants (K_{D}, in μM) at 50% maximal response as determined automatically by the BIAevaluation (GE) software affinity calculation programme. Dissociation constants (K_{D}) are given under the graphs showing each concentration vs RU curve labelled with the appropriate variant.

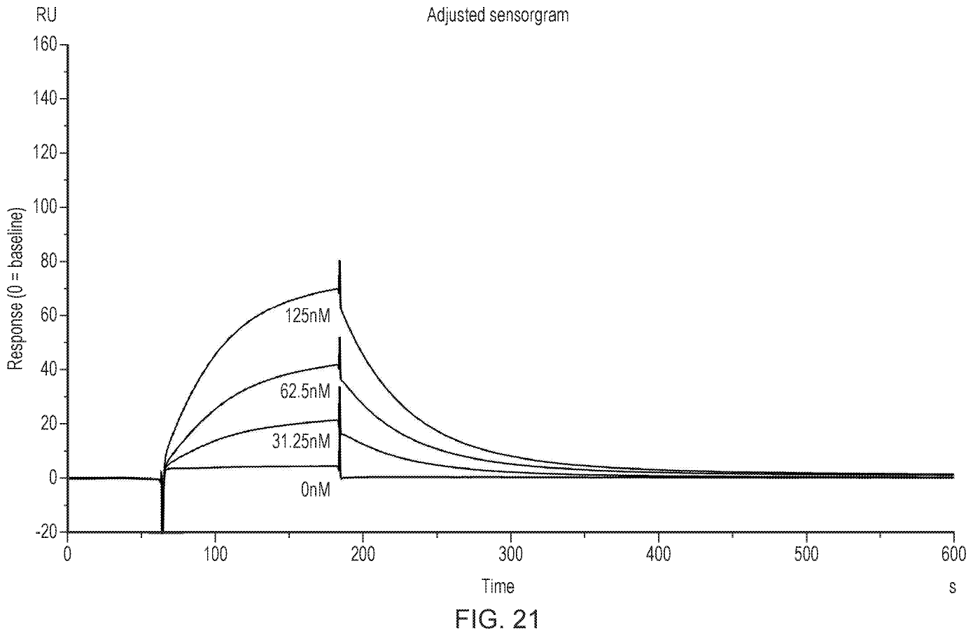

FIG. 21 shows sensorgrams produced in the BIAevaluation software S200 (GE) after injection of 125, 62.5 and 31.25 nM inactive FI (S525A) and WT FLFH onto a mutant C3b (L1109V)-coupled chip (˜900RU, thiol coupled). Time (x-axis, seconds) is plotted versus response (RU) after normalisation by injection over a blank flow cell.

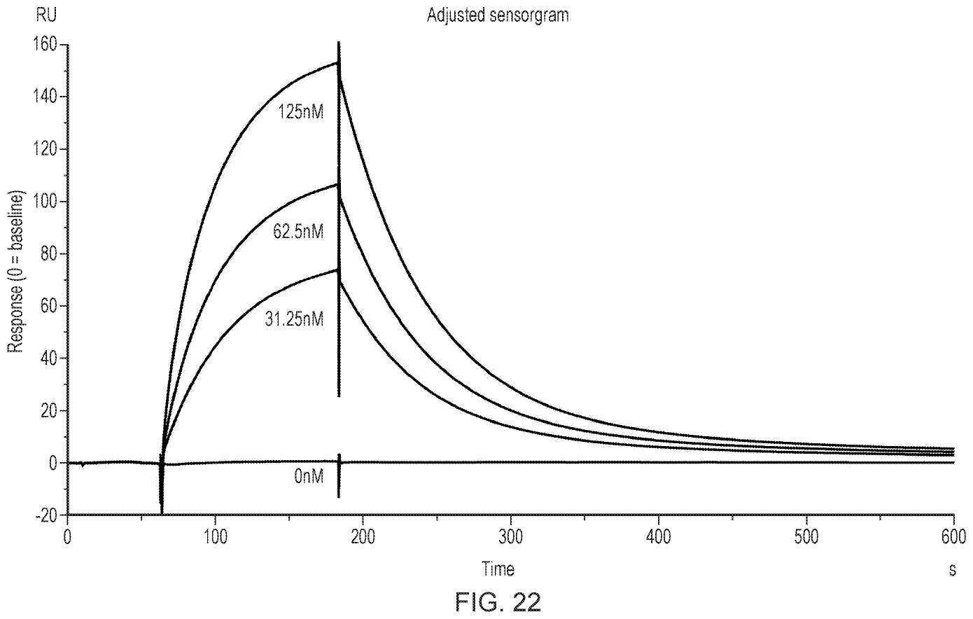

FIG. 22 shows sensorgrams produced in the BIAevaluation software S200 (GE) after injection of 125, 62.5 and 31.25 nM inactive FI (S525A) and WT FLFH (125 nM) onto a WT C3b-coupled chip (˜900RU, thiol coupled). Time (x-axis, seconds) is plotted versus response (RU) after normalisation by injection over a blank flow cell.

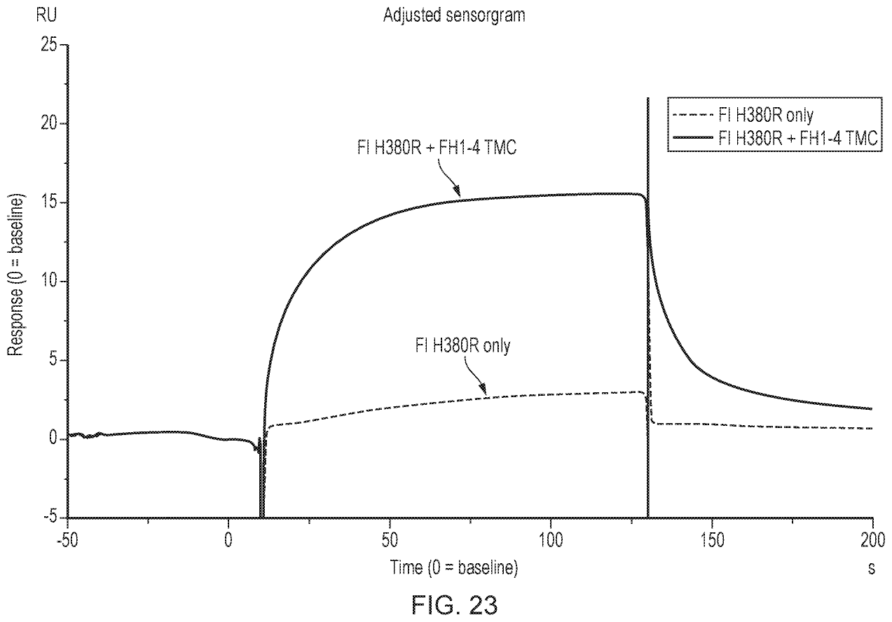

FIG. 23 shows sensorgrams after injection of 125 nM of FI with only an H380R mutation with 125 nM of FH1-4 onto a C3b amine coupled (˜1000RU) CM5 chip using the BIAcore S200, resulting in the building of an AP regulatory trimolecular complex (of ˜15RU) (green line) which did not immediately dissociate when the injection finished after 120 seconds. Meanwhile, injection of 125 nM of H380R FI alone onto the same surface resulted in only a ˜2RU response. Time (x-axis, seconds) is plotted versus response (RU) after normalisation by injection over a blank flow cell.

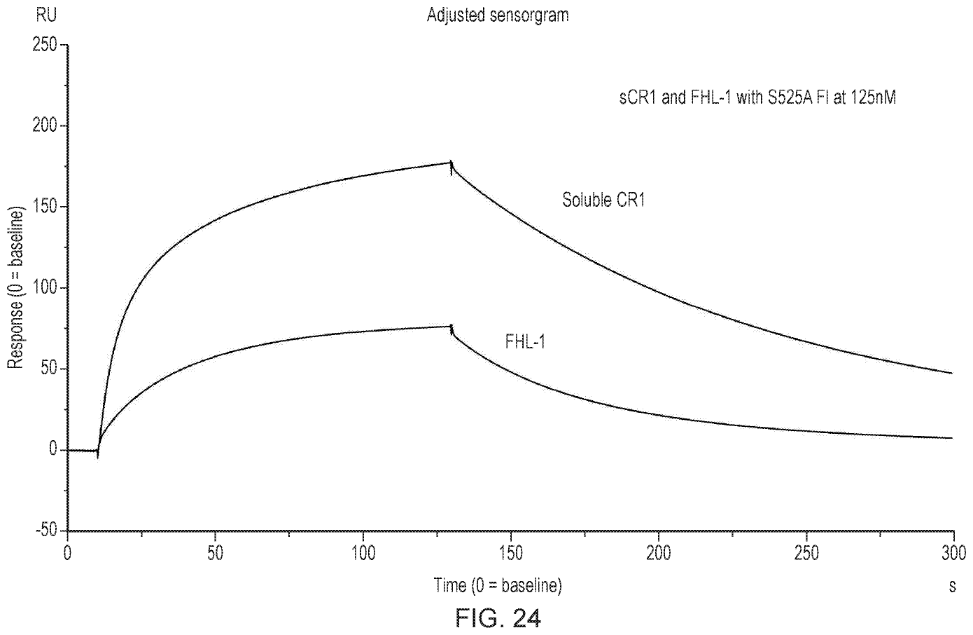

FIG. 24 shows sensorgrams produced in the BIAevaluation software S200 (GE) after injection of 125 nM inactive FI (S525A) and sCR1 or FHL-1 onto a WT C3b-coupled chip (˜1000RU, amine coupled). Time (x-axis, seconds) is plotted versus response (RU) after normalisation by injection over a blank flow cell.

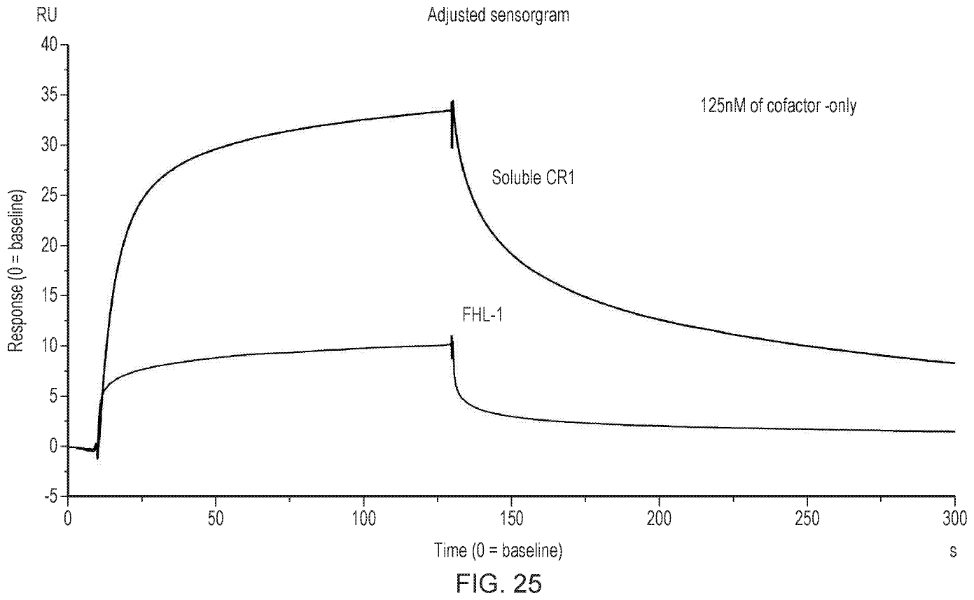

FIG. 25 shows a sensorgram produced in the BIAevaluation software S200 (GE) after injection of 125 nM sCR1 and FHL-1 onto a WT C3b-coupled chip (˜1000RU, amine coupled). Time (x-axis, seconds) is plotted versus response (RU) after normalisation by injection over a blank flow cell.

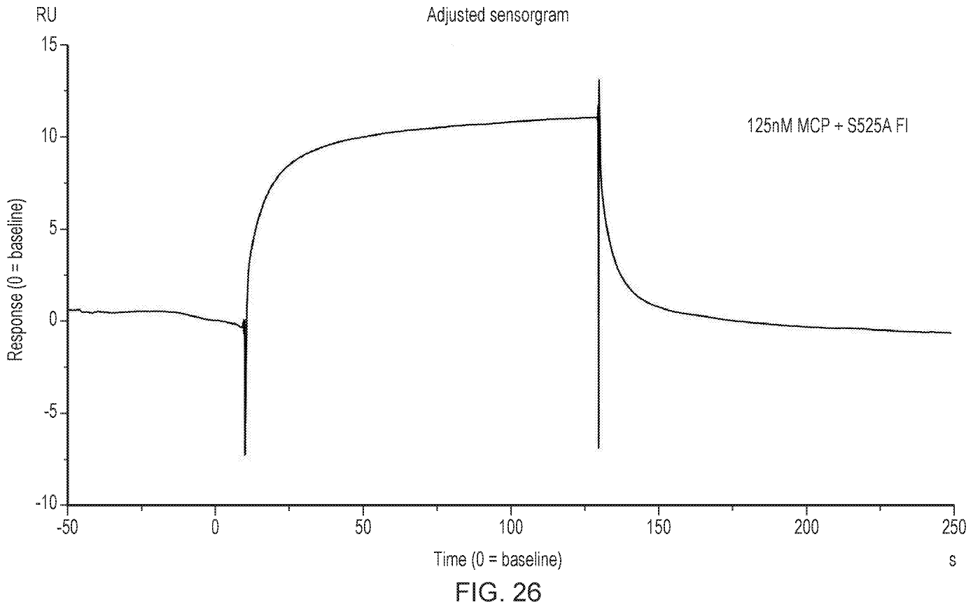

FIG. 26 shows a sensorgram produced in the BIAevaluation software S200 (GE) after injection of 125 nM inactive (S525A) FI and sMCP onto a WT C3b-coupled chip (˜1000RU, amine coupled). Time (x-axis, seconds) is plotted versus response (RU) after normalisation by injection over a blank flow cell.

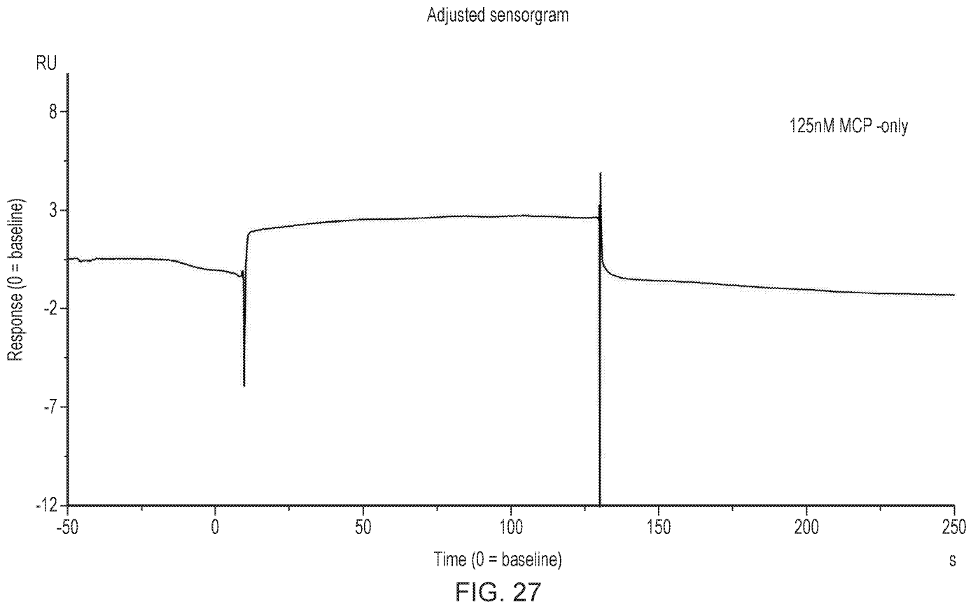

FIG. 27 shows a sensorgram produced in the BIAevaluation software S200 (GE) after injection of 125 nM sMCP onto a WT C3b-coupled chip (˜1000RU, amine coupled). Time (x-axis, seconds) is plotted versus response (RU) after normalisation by injection over a blank flow cell.

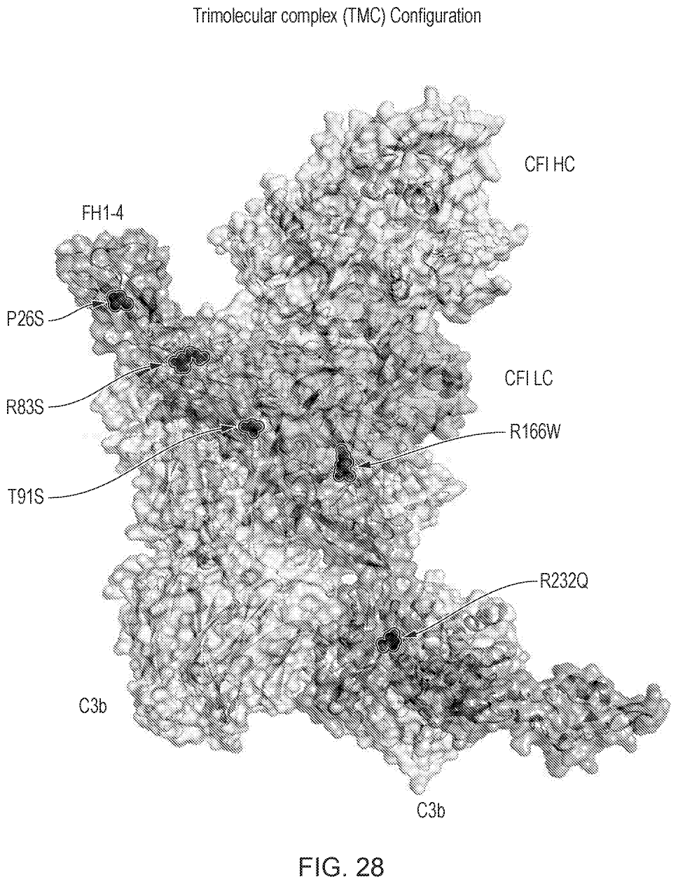

FIG. 28 shows 3D modelling of the FH1-4 variants within the AP regulatory TMC. Modelled are the FH CCP1-4 within the TMC. Highlighted by spheres are the selected FH1-4 variants that were produced for functional analysis, using the on-chip TMC building method.

Sequence Listing
Full Length Human Complement Factor H (FH) (UniprotKB/Swiss-Prot
Accession No: P08603; Version No. P08603.4)
SEQ ID NO: 1
MRLLAKIICLMLWAICVAEDCNELPPRRNTEILTGSWSDQTYPEGTQAIYKCRPGYRSLGNVIMVCRK
GEWVALNPLRKCQKRPCGHPGDTPFGTFTLTGGNVFEYGVKAVYTCNEGYQLLGEINYRECDTDGWTN
DIPICEVVKCLPVTAPENGKIVSSAMEPDREYHFGQAVRFVCNSGYKIEGDEEMHCSDDGFWSKEKPK
CVEISCKSPDVINGSPISQKIIYKENERFQYKCNMGYEYSERGDAVCTESGWRPLPSCEEKSCDNPYI
PNGDYSPLRIKHRTGDEITYQCRNGFYPATRGNTAKCTSTGWIPAPRCTLKPCDYPDIKHGGLYHENM
RRPYFPVAVGKYYSYYCDEHFETPSGSYWDHIHCTQDGWSPAVPCLRKCYFPYLENGYNQNYGRKFVQ
GKSIDVACHPGYALPKAQTTVTCMENGWSPTPRCIRVKTCSKSSIDIENGFISESQYTYALKEKAKYQ
CKLGYVTADGETSGSITCGKDGWSAQPTCIKSCDIPVFMNARTKNDFTWFKLNDTLDYECHDGYESNT
GSTTGSIVCGYNGWSDLPICYERECELPKIDVHLVPDRKKDQYKVGEVLKFSCKPGFTIVGPNSVQCY
HFGLSPDLPICKEQVQSCGPPPELLNGNVKEKTKEEYGHSEVVEYYCNPRFLMKGPNKIQCVDGEWTT
LPVCIVEESTCGDIPELEHGWAQLSSPPYYYGDSVEFNCSESFTMIGHRSITCIHGVWTQLPQCVAID
KLKKCKSSNLIILEEHLKNKKEFDHNSNIRYRCRGKEGWIHTVCINGRWDPEVNCSMAQIQLCPPPPQ
IPNSHNMTTTLNYRDGEKVSVLCQENYLIQEGEEITCKDGRWQSIPLCVEKIPCSQPPQIEHGTINSS
RSSQESYAHGTKLSYTCEGGFRISEENETTCYMGKWSSPPQCEGLPCKSPPEISHGVVAHMSDSYQYG
EEVTYKCFEGFGIDGPAIAKCLGEKWSHPPSCIKTDCLSLPSFENAIPMGEKKDVYKAGEQVTYTCAT
YYKMDGASNVTCINSRWTGRPTCRDTSCVNPPTVQNAYIVSRQMSKYPSGERVRYQCRSPYEMFGDEE
VMCLNGNWTEPPQCKDSTGKCGPPPPIDNGDITSFPLSVYAPASSVEYQCQNLYQLEGNKRITCRNGQ
WSEPPKCLHPCVISREIMENYNIALRWTAKQKLYSRTGESVEFVCKRGYRLSSRSHTLRTTCWDGKLE
YPTCAKR
Recombinant Human Complement Factor H CCPs 1-4 (6x His-tagged)
(FH 1-4)
SEQ ID NO: 2
EKREAEAAGEQKLISEEDLEDCNELPPRRNTEILTGSWSDQTYPEGTQAIYKCRPGYRSLGNVIMVCR
KGEWVALNPLRKCQKRPCGHPGDTPFGTFTLTGGNVFEYGVKAVYTCNEGYQLLGEINYRECDTDGWT
NDIPICEVVKCLPVTAPENGKIVSSAMEPDREYHFGQAVRFVCNSGYKIEGDEEMHCSDDGFWSKEKP
KCVEISCKSPDVINGSPISQKIIYKENERFQYKCNMGYEYSERGDAVCTESGWRPLPSCEHHHHHH
Human Precursor Complement Factor|(FI) (NCBI Accession No.
NP_000195; Version No. NP_000195.3)
SEQ ID NO: 3
MKLLHVFLLFLCFHLRFCKVTYTSQEDLVEKKCLAKKYTHLSCDKVFCQPWORCIEGTCVCKLPYQCP
KNGTAVCATNRRSFPTYCQQKSLECLHPGTKFLNNGTCTAEGKFSVSLKHGNTDSEGIVEVKLVDQDK
TMFICKSSWSMREANVACLDLGFQQGADTQRRFKLSDLSINSTECLHVHCRGLETSLAECTFTKRRTM
GYQDFADVVCYTQKADSPMDDFFQCVNGKYISQMKACDGINDCGDQSDELCCKACQGKGFHCKSGVCI
PSQYQCNGEVDCITGEDEVGCAGFASVTQEETEILTADMDAERRRIKSLLPKLSCGVKNRMHIRRKRI
VGGKRAQLGDLPWQVAIKDASGITCGGIYIGGCWILTAAHCLRASKTHRYQIWTTVVDWIHPDLKRIV
IEYVDRIIFHENYNAGTYQNDIALIEMKKDGNKKDCELPRSIPACVPWSPYLFQPNDTCIVSGWGREK
DNERVESLOWGEVKLISNCSKFYGNRFYEKEMECAGTYDGSIDACKGDSGGPLVCMDANNVTYVWGVV
SWGENCGKPEFPGVYTKVANYFDWISYHVGRPFISQYNV
Complement Factor|Heavy Chain (HC) (UniprotKB/Swiss-Prot
Accession No: Q6LAM1; Version No. Q6LAM1)
SEQ ID NO: 4
KVTYTSQEDLVEKKCLAKKYTHLSCDKVFCQPWQRCIEGTCVCKLPYQCPKNGTAVCATNRRSFPTYC
QQKSLECLHPGTKFLNNGTCTAEGKFSVSLKHGNTDSEGIVEVKLVDQDKTMFICKSSWSMREANVAC
LDLGFQQGADTQRRFKLSDLSINSTECLHVHCRGLETSLAECTFTKRRTMGYQDFADVVCYTQKADSP
MDDFFQCVNGKYISQMKACDGINDCGDQSDELCCKACQGKGFHCKSGVCIPSQYQCNGEVDCITGEDE
VGCAGFASVAQEETEILTADMDAERRRIKSLLPKLSCGVKNRMHI
Complement Factor I Light Chain (LC) (UniprotKB/Swiss-Prot
Accession No. Q6LAMO; Version No. Q6LAM0)
SEQ ID NO. 5
IVGGKRAQLGDLPWQVAIKDASGITCGGIYIGGCWILTAAHCLRASKTHRYQIWTTVVDWIHPDLKRI
VIEYVDRIIFHENYNAGTYQNDIALIEMKKDGNKKDCELPRSIPACVPWSPYLFQPNDTCIVSGWGRE
KDNERVESLOWGEVKLISNCSKFYGNRFYEKEMECAGTYDGSIDACKGDSGGPLVCMDANNVTYVWG
VVSWGENCGKPEFPGVYTKVANYFDWISYHVGRPFISQYNV
FI Linker Sequence
SEQ ID NO. 6
RRKR
Recombinant S525A Precursor Human Complement Factor I
SEQ ID NO: 7
MKLLHVFLLFLCFHLRFCKVTYTSQEDLVEKKCLAKKYTHLSCDKVFCQPWQRCIEGTCVCKLPYQCP
KNGTAVCATNRRSFPTYCQQKSLECLHPGTKFLNNGTCTAEGKFSVSLKHGNTDSEGIVEVKLVDQDK
TMFICKSSWSMREANVACLDLGFQQGADTQRRFKLSDLSINSTECLHVHCRGLETSLAECTFTKRRTM
GYQDFADVVCYTQKADSPMDDFFQCVNGKYISQMKACDGINDCGDQSDELCCKACQGKGFHCKSGVCI
PSQYQCNGEVDCITGEDEVGCAGFASVTQEETEILTADMDAERRRIKSLLPKLSCGVKNRMHIRRKRI
VGGKRAQLGDLPWQVAIKDASGITCGGIYIGGCWILTAAHCLRASKTHRYQIWTTVVDWIHPDLKRIV
IEYVDRIIFHENYNAGTYQNDIALIEMKKDGNKKDCELPRSIPACVPWSPYLFQPNDTCIVSGWGREK
DNERVFSLOWGEVKLISNCSKFYGNRFYEKEMECAGTYDGSIDACKGDAGGPLVCMDANNVTYVWGVV
SWGENCGKPEFPGVYTKVANYFDWISYHVGRPFISQYNV
Complement Factor I Light Chain (LC) derived from S525A
precursor Human Complement Factor I
SEQ ID NO: 8
IVGGKRAQLGDLPWQVAIKDASGITCGGIYIGGCWILTAAHCLRASKTHRYQIWTTVVDWIHPDLKRI
VIEYVDRIIFHENYNAGTYQNDIALIEMKKDGNKKDCELPRSIPACVPWSPYLFQPNDTCIVSGWGRE
KDNERVESLOWGEVKLISNCSKFYGNRFYEKEMECAGTYDGSIDACKGDAGGPLVCMDANNVTYVWG
VVSWGENCGKPEFPGVYTKVANYFDWISYHVGRPFISQYNV
Human C3b (UniprotKB/Swiss-Prot Accession No. P01024;
Version No. P01024.2)
SEQ ID NO: 9
MGPTSGPSLLLLLLTHLPLALGSPMYSIITPNILRLESEETMVLEAHDAQGDVPVTVTVHDFPGKKLV
LSSEKTVLTPATNHMGNVTFTIPANREFKSEKGRNKFVTVQATFGTQVVEKVVLVSLOSGYLFIQTDK
TIYTPGSTVLYRIFTVNHKLLPVGRTVMVNIENPEGIPVKQDSLSSQNQLGVLPLSWDIPELVNMGQW
KIRAYYENSPQQVFSTEFEVKEYVLPSFEVIVEPTEKFYYIYNEKGLEVTITARFLYGKKVEGTAFVI
FGIQDGEQRISLPESLKRIPIEDGSGEVVLSRKVLLDGVONPRAEDLVGKSLYVSATVILHSGSDMVQ
AERSGIPIVTSPYQIHFTKTPKYFKPGMPFDLMVFVTNPDGSPAYRVPVAVQGEDTVQSLTQGDGVAK
LSINTHPSQKPLSITVRTKKQELSEAEQATRTMQALPYSTVGNSNNYLHLSVLRTELRPGETLNVNFL
LRMDRAHEAKIRYYTYLIMNKGRLLKAGRQVREPGQDLVVLPLSITTDFIPSFRLVAYYTLIGASGQR
EVVADSVWVDVKDSCVGSLVVKSGQSEDRQPVPGQQMTLKIEGDHGARVVLVAVDKGVFVLNKKNKLT
QSKIWDVVEKADIGCTPGSGKDYAGVFSDAGLTFTSSSGQQTAQRAELQCPQPAARRRRSVOLTEKRM
DKVGKYPKELRKCCEDGMRENPMRFSCQRRTRFISLGEACKKVFLDCCNYITELRRQHARASHLGLAR
SNLDEDIIAEENIVSRSEFPESWLWNVEDLKEPPKNGISTKLMNIFLKDSITTWEILAVSMSDKKGIC
VADPFEVTVMQDFFIDLRLPYSVVRNEQVEIRAVLYNYRQNQELKVRVELLHNPAFCSLATTKRRHQQ
TVTIPPKSSLSVPYVIVPLKTGLQEVEVKAAVYHHFISDGVRKSLKVVPEGIRMNKTVAVRTLDPERL
GREGVQKEDIPPADLSDQVPDTESETRILLQGTPVAQMTEDAVDAERLKHLIVTPSGCGEQNMIGMTP
TVIAVHYLDETEQWEKFGLEKRQGALELIKKGYTQQLAFRQPSSAFAAFVKRAPSTWLTAYVVKVFSL
AVNLIAIDSQVLCGAVKWLILEKQKPDGVFQEDAPVIHQEMIGGLRNNNEKDMALTAFVLISLQEAKD
ICEEQVNSLPGSITKAGDFLEANYMNLQRSYTVAIAGYALAQMGRLKGPLLNKFLTTAKDKNRWEDPG
KQLYNVEATSYALLALLQLKDFDFVPPVVRWLNEQRYYGGGYGSTQATFMVFQALAQYOKDAPDHQEL
NLDVSLQLPSRSSKITHRIHWESASLLRSEETKENEGFTVTAEGKGQGTLSVVTMYHAKAKDQLTCNK
FDLKVTIKPAPETEKRPQDAKNTMILEICTRYRGDQDATMSILDISMMTGFAPDTDDLKQLANGVDRY
ISKYELDKAFSDRNTLIIYLDKVSHSEDDCLAFKVHQYFNVELIQPGAVKVYAYYNLEESCTRFYHPE
KEDGKLNKLCRDELCRCAEENCFIQKSDDKVTLEERLDKACEPGVDYVYKTRLVKVQLSNDFDEYIMA
IEQTIKSGSDEVQVGQQRTFISPIKCREALKLEEKKHYLMWGLSSDFWGEKPNLSYIIGKDTWVEHWP
EEDECQDEENQKQCQDLGAFTESMVVFGCPN
Human Complement Receptor 1 (CR1) (UniprotKB/Swiss-Prot
Accession No. P17927, Version No. P17927.3)
SEQ ID NO: 10
MGASSPRSPEPVGPPAPGLPFCCGGSLLAVVVLLALPVAWGQCNAPEWLPFARPTNLTDEFEFPIGTY
LNYECRPGYSGRPFSIICLKNSVWTGAKDRCRRKSCRNPPDPVNGMVHVIKGIQFGSQIKYSCTKGYR
LIGSSSATCIISGDTVIWDNETPICDRIPCGLPPTITNGDFISTNRENFHYGSVVTYRCNPGSGGRKV
FELVGEPSIYCTSNDDQVGIWSGPAPQCIIPNKCTPPNVENGILVSDNRSLFSLNEVVEFRCQPGFVM
KGPRRVKCQALNKWEPELPSCSRVCQPPPDVLHAERTQRDKDNFSPGQEVFYSCEPGYDLRGAASMRC
TPQGDWSPAAPTCEVKSCDDFMGQLLNGRVLFPVNLQLGAKVDFVCDEGFQLKGSSASYCVLAGMESL
WNSSVPVCEQIFCPSPPVIPNGRHTGKPLEVFPFGKTVNYTCDPHPDRGTSFDLIGESTIRCTSDPQG
NGVWSSPAPRCGILGHCQAPDHFLFAKLKTQTNASDFPIGTSLKYECRPEYYGRPFSITCLDNLVWSS
PKDVCKRKSCKTPPDPVNGMVHVITDIQVGSRINYSCTTGHRLIGHSSAECILSGNAAHWSTKPPICQ
RIPCGLPPTIANGDFISTNRENFHYGSVVTYRCNPGSGGRKVFELVGEPSIYCTSNDDQVGIWSGPAP
QCIIPNKCTPPNVENGILVSDNRSLFSLNEVVEFRCQPGFVMKGPRRVKCQALNKWEPELPSCSRVCQ
PPPDVLHAERTQRDKDNFSPGQEVFYSCEPGYDLRGAASMRCTPQGDWSPAAPTCEVKSCDDFMGQLL
NGRVLFPVNLQLGAKVDFVCDEGFQLKGSSASYCVLAGMESLWNSSVPVCEQIFCPSPPVIPNGRHTG
KPLEVFPFGKAVNYTCDPHPDRGTSFDLIGESTIRCTSDPQGNGVWSSPAPRCGILGHCQAPDHFLFA
KLKTQTNASDFPIGTSLKYECRPEYYGRPFSITCLDNLVWSSPKDVCKRKSCKTPPDPVNGMVHVITD
IQVGSRINYSCTTGHRLIGHSSAECILSGNTAHWSTKPPICQRIPCGLPPTIANGDFISTNRENFHYG
SVVTYRCNLGSRGRKVFELVGEPSIYCTSNDDQVGIWSGPAPQCIIPNKCTPPNVENGILVSDNRSLF
SLNEVVEFRCQPGFVMKGPRRVKCQALNKWEPELPSCSRVCQPPPEILHGEHTPSHQDNFSPGQEVFY
SCEPGYDLRGAASLHCTPQGDWSPEAPRCAVKSCDDFLGQLPHGRVLFPLNLQLGAKVSFVCDEGFRL
KGSSVSHCVLVGMRSLWNNSVPVCEHIFCPNPPAILNGRHTGTPSGDIPYGKEISYTCDPHPDRGMTF
NLIGESTIRCTSDPHGNGVWSSPAPRCELSVRAGHCKTPEQFPFASPTIPINDFEFPVGTSLNYECRP
GYFGKMFSISCLENLVWSSVEDNCRRKSCGPPPEPENGMVHINTDTQFGSTVNYSCNEGERLIGSPST
TCLVSGNNVTWDKKAPICEIISCEPPPTISNGDFYSNNRTSFHNGTVVTYQCHTGPDGEQLFELVGER
SIYCTSKDDQVGVWSSPPPRCISTNKCTAPEVENAIRVPGNRSFFSLTEIIRFRCQPGFVMVGSHTVQ
CQTNGRWGPKLPHCSRVCQPPPEILHGEHTLSHQDNFSPGQEVFYSCEPSYDLRGAASLHCTPQGDWS
PEAPRCTVKSCDDFLGQLPHGRVLLPLNLQLGAKVSFVCDEGFRLKGRSASHCVLAGMKALWNSSVPV
CEQIFCPNPPAILNGRHTGTPFGDIPYGKEISYACDTHPDRGMTFNLIGESSIRCTSDPQGNGVWSSP
APRCELSVPAACPHPPKIQNGHYIGGHVSLYLPGMTISYICDPGYLLVGKGFIFCTDQGIWSQLDHYC
KEVNCSFPLFMNGISKELEMKKVYHYGDYVTLKCEDGYTLEGSPWSQCQADDRWDPPLAKCTSRTHDA
LIVGTLSGTIFFILLIIFLSWIILKHRKGNNAHENPKEVAIHLHSQGGSSVHPRTLQTNEENSRVLP
Recombinant Soluble Complement Receptor 1 (lacking the
transmembrane domain sequence (1972-2039) and lacking
the signal peptide (1-41))
SEQ ID NO: 11
QCNAPEWLPFARPTNLTDEFEFPIGTYLNYECRPGYSGRPFSIICLKNSVWTGAKDRCRRKSCRNPPD
PVNGMVHVIKGIQFGSQIKYSCTKGYRLIGSSSATCIISGDTVIWDNETPICDRIPCGLPPTITNGDF
ISTNRENFHYGSVVTYRCNPGSGGRKVFELVGEPSIYCTSNDDQVGIWSGPAPQCIIPNKCTPPNVEN
GILVSDNRSLFSLNEVVEFRCQPGFVMKGPRRVKCQALNKWEPELPSCSRVCQPPPDVLHAERTQRDK
DNFSPGQEVFYSCEPGYDLRGAASMRCTPQGDWSPAAPTCEVKSCDDFMGQLLNGRVLFPVNLQLGAK
VDFVCDEGFQLKGSSASYCVLAGMESLWNSSVPVCEQIFCPSPPVIPNGRHTGKPLEVFPFGKTVNYT
CDPHPDRGTSFDLIGESTIRCTSDPQGNGVWSSPAPRCGILGHCQAPDHFLFAKLKTQTNASDFPIGT
SLKYECRPEYYGRPFSITCLDNLVWSSPKDVCKRKSCKTPPDPVNGMVHVITDIQVGSRINYSCTTGH
RLIGHSSAECILSGNAAHWSTKPPICQRIPCGLPPTIANGDFISTNRENFHYGSVVTYRCNPGSGGRK
VFELVGEPSIYCTSNDDQVGIWSGPAPQCIIPNKCTPPNVENGILVSDNRSLFSLNEVVEFRCQPGFV
MKGPRRVKCQALNKWEPELPSCSRVCQPPPDVLHAERTQRDKDNFSPGQEVFYSCEPGYDLRGAASMR
CTPQGDWSPAAPTCEVKSCDDFMGQLLNGRVLFPVNLQLGAKVDFVCDEGFQLKGSSASYCVLAGMES
LWNSSVPVCEQIFCPSPPVIPNGRHTGKPLEVFPFGKAVNYTCDPHPDRGTSFDLIGESTIRCTSDPQ
GNGVWSSPAPRCGILGHCQAPDHFLFAKLKTQTNASDFPIGTSLKYECRPEYYGRPFSITCLDNLVWS
SPKDVCKRKSCKTPPDPVNGMVHVITDIQVGSRINYSCTTGHRLIGHSSAECILSGNTAHWSTKPPIC
QRIPCGLPPTIANGDFISTNRENFHYGSVVTYRCNLGSRGRKVFELVGEPSIYCTSNDDQVGIWSGPA
PQCIIPNKCTPPNVENGILVSDNRSLFSLNEVVEFRCQPGFVMKGPRRVKCQALNKWEPELPSCSRVC
QPPPEILHGEHTPSHQDNFSPGQEVFYSCEPGYDLRGAASLHCTPQGDWSPEAPRCAVKSCDDFLGQL
PHGRVLFPLNLQLGAKVSFVCDEGFRLKGSSVSHCVLVGMRSLWNNSVPVCEHIFCPNPPAILNGRHT
GTPSGDIPYGKEISYTCDPHPDRGMTFNLIGESTIRCTSDPHGNGVWSSPAPRCELSVRAGHCKTPEQ
FPFASPTIPINDFEFPVGTSLNYECRPGYFGKMFSISCLENLVWSSVEDNCRRKSCGPPPEPENGMVH
INTDTQFGSTVNYSCNEGFRLIGSPSTTCLVSGNNVTWDKKAPICEIISCEPPPTISNGDFYSNNRTS
FHNGTVVTYQCHTGPDGEQLFELVGERSIYCTSKDDQVGVWSSPPPRCISTNKCTAPEVENAIRVPGN
RSFFSLTEIIRFRCQPGFVMVGSHTVQCQTNGRWGPKLPHCSRVCQPPPEILHGEHTLSHQDNFSPGQ
EVFYSCEPSYDLRGAASLHCTPQGDWSPEAPRCTVKSCDDFLGQLPHGRVLLPLNLQLGAKVSFVCDE
GFRLKGRSASHCVLAGMKALWNSSVPVCEQIFCPNPPAILNGRHTGTPFGDIPYGKEISYACDTHPDR
GMTFNLIGESSIRCTSDPQGNGVWSSPAPRCELSVPAACPHPPKIQNGHYIGGHVSLYLPGMTISYIC
DPGYLLVGKGFIFCTDQGIWSQLDHYCKEVNCSFPLFMNGISKELEMKKVYHYGDYVTLKCEDGYTLE
GSPWSQCQADDRWDPPLAKCTSRTHD
Human Mature Membrane Cofactor Protein (MCP) (UniprotKB/Swiss-
Prot Accession No. P15529; Version No. P15529.3)
SEQ ID NO: 12
MEPPGRRECPFPSWRFPGLLLAAMVLLLYSFSDACEEPPTFEAMELIGKPKPYYEIGERVDYKCKKGY
FYIPPLATHTICDRNHTWLPVSDDACYRETCPYIRDPLNGQAVPANGTYEFGYQMHFICNEGYYLIGE
EILYCELKGSVAIWSGKPPICEKVLCTPPPKIKNGKHTFSEVEVFEYLDAVTYSCDPAPGPDPFSLIG
ESTIYCGDNSVWSRAAPECKVVKCRFPVVENGKQISGFGKKFYYKATVMFECDKGFYLDGSDTIVCDS
NSTWDPPVPKCLKVLPPSSTKPPALSHSVSTSSTTKSPASSASGPRPTYKPPVSNYPGYPKPEEGILD
SLDVWVIAVIVIAIVVGVAVICVVPYRYLQRRKKKGTYLTDETHREVKFTSL
Recombinant Human Mature Membrane Cofactor Protein lacking the
signal peptide (amino acids 1-34)
SEQ ID NO: 13
CEEPPTFEAMELIGKPKPYYEIGERVDYKCKKGYFYIPPLATHTICDRNHTWLPVSDDACYRETCPYI
RDPLNGQAVPANGTYEFGYQMHFICNEGYYLIGEEILYCELKGSVAIWSGKPPICEKVLCTPPPKIKN
GKHTFSEVEVFEYLDAVTYSCDPAPGPDPFSLIGESTIYCGDNSVWSRAAPECKVVKCRFPVVENGKQ
ISGFGKKFYYKATVMFECDKGFYLDGSDTIVCDSNSTWDPPVPKCLKVLPPSSTKPPALSHSVSTSST
TKSPASSASGPRPTYKPPVSNYPGYPKPEEGILDSLDVWVIAVIVIAIVVGVAVICVVPYRYLQRRKK
KGTYLTDETHREVKFTSL
Human Factor H-like protein 1 (UniprotKB/Swiss-Prot Accession
No. Q03591, Version No. Q03591.2)
SEQ ID NO: 14
MRLLAKIICLMLWAICVAEDCNELPPRRNTEILTGSWSDQTYPEGTQAIYKCRPGYRSLGNVIMVCRK
GEWVALNPLRKCQKRPCGHPGDTPFGTFTLTGGNVFEYGVKAVYTCNEGYQLLGEINYRECDTDGWTN
DIPICEVVKCLPVTAPENGKIVSSAMEPDREYHFGQAVRFVCNSGYKIEGDEEMHCSDDGFWSKEKPK
CVEISCKSPDVINGSPISQKIIYKENERFQYKCNMGYEYSERGDAVCTESGWRPLPSCEEKSCDNPYI
PNGDYSPLRIKHRTGDEITYQCRNGFYPATRGNTAKCTSTGWIPAPRCTLKPCDYPDIKHGGLYHENM
RRPYFPVAVGKYYSYYCDEHFETPSGSYWDHIHCTQDGWSPAVPCLRKCYFPYLENGYNQNYGRKFVQ
GKSIDVACHPGYALPKAQTTVTCMENGWSPTPRCIRVSFTL
Recombinant Factor H-like protein 1 (FHL-1) lacking the
signal peptide (amino acids 1-18)
SEQ ID NO: 15
EDCNELPPRRNTEILTGSWSDQTYPEGTQAIYKCRPGYRSLGNVIMVCRKGEWVALNPLRKCQKRPCG
HPGDTPFGTFTLTGGNVFEYGVKAVYTCNEGYQLLGEINYRECDTDGWINDIPICEVVKCLPVTAPEN
GKIVSSAMEPDREYHFGQAVRFVCNSGYKIEGDEEMHCSDDGFWSKEKPKCVEISCKSPDVINGSPIS
QKIIYKENERFQYKCNMGYEYSERGDAVCTESGWRPLPSCEEKSCDNPYIPNGDYSPLRIKHRTGDEI
TYQCRNGFYPATRGNTAKCTSTGWIPAPRCTLKPCDYPDIKHGGLYHENMRRPYFPVAVGKYYSYYCD
EHFETPSGSYWDHIHCTQDGWSPAVPCLRKCYFPYLENGYNQNYGRKFVQGKSIDVACHPGYALPKAQ
TTVTCMENGWSPTPRCIRVSFTL
Recombinant H380R Precursor Human Complement Factor I
SEQ ID NO: 16
MKLLHVFLLFLCFHLRFCKVTYTSQEDLVEKKCLAKKYTHLSCDKVFCQPWQRCIEGTCVCKLPYQCP
KNGTAVCATNRRSFPTYCQQKSLECLHPGTKFLNNGTCTAEGKFSVSLKHGNTDSEGIVEVKLVDQDK
TMFICKSSWSMREANVACLDLGFQQGADTQRRFKLSDLSINSTECLHVHCRGLETSLAECTFTKRRTM
GYQDFADVVCYTQKADSPMDDFFQCVNGKYISQMKACDGINDCGDQSDELCCKACQGKGFHCKSGVCI
PSQYQCNGEVDCITGEDEVGCAGFASVTQEETEILTADMDAERRRIKSLLPKLSCGVKNRMHIRRKRI
VGGKRAQLGDLPWQVAIKDASGITCGGIYIGGCWILTAARCLRASKTHRYQIWTTVVDWIHPDLKRIV
IEYVDRIIFHENYNAGTYQNDIALIEMKKDGNKKDCELPRSIPACVPWSPYLFQPNDTCIVSGWGREK
DNERVESLOWGEVKLISNCSKFYGNRFYEKEMECAGTYDGSIDACKGDSGGPLVCMDANNVTYVWGVV
SWGENCGKPEFPGVYTKVANYFDWISYHVGRPFISQYNV
Complement Factor I Light Chain (LC) derived from H380R
precursor Human Complement Factor I
SEQ ID NO: 17
IVGGKRAQLGDLPWQVAIKDASGITCGGIYIGGCWILTAARCLRASKTHRYQIWTTVVDWIHPDLKRI
VIEYVDRIIFHENYNAGTYQNDIALIEMKKDGNKKDCELPRSIPACVPWSPYLFQPNDTCIVSGWGRE
KDNERVESLOWGEVKLISNCSKFYGNRFYEKEMECAGTYDGSIDACKGDSGGPLVCMDANNVTYVWG
VVSWGENCGKPEFPGVYTKVANYFDWISYHVGRPFISQYNV
Human C4b (UniprotKB/Swiss-Prot Accession No. P0C0L5;
Version No. 2 (of the sequence)
SEQ ID NO: 18
MRLLWGLIWASSFFTLSLQKPRLLLFSPSVVHLGVPLSVGVQLQDVPRGQVVKGSVFLRNPSRNNVPC
SPKVDFTLSSERDFALLSLQVPLKDAKSCGLHQLLRGPEVQLVAHSPWLKDSLSRTTNIQGINLLESS
RRGHLFLQTDQPIYNPGQRVRYRVFALDQKMRPSTDTITVMVENSHGLRVRKKEVYMPSSIFQDDFVI
PDISEPGTWKISARFSDGLESNSSTQFEVKKYVLPNFEVKITPGKPYILTVPGHLDEMQLDIQARYIY
GKPVQGVAYVRFGLLDEDGKKTFFRGLESQTKLVNGQSHISLSKAEFQDALEKLNMGITDLQGLRLYV
AAAIIESPGGEMEEAELTSWYFVSSPFSLDLSKTKRHLVPGAPFLLQALVREMSGSPASGIPVKVSAT
VSSPGSVPEVQDIQQNTDGSGQVSIPIIIPQTISELQLSVSAGSPHPAIARLTVAAPPSGGPGELSIE
RPDSRPPRVGDTLNLNLRAVGSGATFSHYYYMILSRGQIVFMNREPKRTLTSVSVFVDHHLAPSFYFV
AFYYHGDHPVANSLRVDVQAGACEGKLELSVDGAKQYRNGESVKLHLETDSLALVALGALDTALYAAG
SKSHKPLNMGKVFEAMNSYDLGCGPGGGDSALQVFQAAGLAFSDGDQWTLSRKRLSCPKEKTTRKKRN
VNFQKAINEKLGQYASPTAKRCCQDGVTRLPMMRSCEQRAARVQQPDCREPFLSCCQFAESLRKKSRD
KGQAGLQRALEILQEEDLIDEDDIPVRSFFPENWLWRVETVDRFQILTLWLPDSLTTWEIHGLSLSKT
KGLCVATPVQLRVFREFHLHLRLPMSVRRFEQLELRPVLYNYLDKNLTVSVHVSPVEGLCLAGGGGLA
QQVLVPAGSARPVAFSVVPTAATAVSLKVVARGSFEFPVGDAVSKVLQIEKEGAIHREELVYELNPLD
HRGRTLEIPGNSDPNMIPDGDENSYVRVTASDPLDTLGSEGALSPGGVASLLRLPRGCGEQTMIYLAP
TLAASRYLDKTEQWSTLPPETKDHAVDLIQKGYMRIQQFRKADGSYAAWLSRGSSTWLTAFVLKVLSL
AQEQVGGSPEKLQETSNWLLSQQQADGSFQDLSPVIHRSMQGGLVGNDETVALTAFVTIALHHGLAVE
QDEGAEPLKQRVEASISKASSFLGEKASAGLIGAHAAAITAYALTLTKAPADLRGVAHNNLMAMAQET
GDNLYWGSVTGSQSNAVSPTPAPRNPSDPMPQAPALWIETTAYALLHLLLHEGKAEMADQAAAWLTRQ
GSFQGGFRSTQDTVIALDALSAYWIASHTTEERGLNVTLSSTGRNGFKSHALQLNNRQIRGLEEELQF
SLGSKINVKVGGNSKGTLKVLRTYNVLDMKNTTCQDLQIEVTVKGHVEYTMEANEDYEDYEYDELPAK
DDPDAPLQPVTPLQLFEGRRNRRRREAPKVVEEQESRVHYTVCIWRNGKVGLSGMAIADVTLLSGFHA
LRADLEKLTSLSDRYVSHFETEGPHVLLYFDSVPTSRECVGFEAVQEVPVGLVQPASATLYDYYNPER
RCSVFYGAPSKSRLLATLCSAEVCQCAEGKCPRQRRALERGLQDEDGYRMKFACYYPRVEYGFQVKVL
REDSRAAFRLFETKITQVLHFTKDVKAAANQMRNFLVRASCRLRLEPGKEYLIMGLDGATYDLEGHPQ
YLLDSNSWIEEMPSERLCRSTRQRAACAQLNDFLQEYGTQGCQV
Human C4b Binding Protein (C4bp) alpha chain (UniprotKB/
Swiss-Prot Accession No. P04003; Version No. P04003.v2
(of the sequence))
SEQ ID NO: 19
MHPPKTPSGALHRKRKMAAWPFSRLWKVSDPILFQMTLIAALLPAVLGNCGPPPILSFAAPMDITLTE
TRFKTGTTLKYTCLPGYVRSHSTQTLTCNSDGEWVYNTFCIYKRCRHPGELRNGQVEIKTDLSFGSQI
EFSCSEGFFLIGSTTSRCEVQDRGVGWSHPLPQCEIVKCKPPPDIRNGRHSGEENFYAYGFSVTYSCD
PRFSLLGHASISCTVENETIGVWRPSPPTCEKITCRKPDVSHGEMVSGFGPIYNYKDTIVFKCQKGFV
LRGSSVIHCDADSKWNPSPPACEPNSCINLPDIPHASWETYPRPTKEDVYVVGTVLRYRCHPGYKPTT
DEPTTVICQKNLRWTPYQGCEALCCPEPKLNNGEITQHRKSRPANHCVYFYGDEISFSCHETSRFSAI
CQGDGTWSPRTPSCGDICNFPPKIAHGHYKQSSSYSFFKEEIIYECDKGYILVGQAKLSCSYSHWSAP
APQCKALCRKPELVNGRLSVDKDQYVEPENVTIQCDSGYGVVGPQSITCSGNRTWYPEVPKCEW
ETPEGCEQVLTGKRLMQCLPNPEDVKMALEVYKLSLEIEQLELORDSARQSTLDKEL
Human C4b Binding Protein (C4bp) beta chain (UniprotKB/
Swiss-Prot Accession No. P20851; Version No. P20851.v1
(of the sequence))
SEQ ID NO: 20
MFFWCACCLMVAWRVSASDAEHCPELPPVDNSIFVAKEVEGQILGTYVCIKGYHLVGKKTLFCNASKE
WDNTTTECRLGHCPDPVLVNGEFSSSGPVNVSDKITFMCNDHYILKGSNRSQCLEDHTWAPPFPICKS
RDCDPPGNPVHGYFEGNNFTLGSTISYYCEDRYYLVGVQEQQCVDGEWSSALPVCKLIQEAPKPECEK
ALLAFQESKNLCEAMENEMQQLKESGMTMEELKYSLELKKAELKAKLL

### DETAILED DESCRIPTION OF CERTAIN EMBODIMENTS OF THE DISCLOSURE

The practice of embodiments of the present invention employs, unless otherwise indicated, conventional techniques of chemistry, molecular biology, pharmaceutical formulation, pharmacology and medicine, which are within the skill of those working in the art.

Most general chemistry techniques can be found in Comprehensive Heterocyclic Chemistry IF (Katritzky et al., 1996, published by Pergamon Press); Comprehensive Organic Functional Group Transformations (Katritzky et al., 1995, published by Pergamon Press); Comprehensive Organic Synthesis (Trost et al., 1991, published by Pergamon); Heterocyclic Chemistry (Joule et al. published by Chapman & Hall); Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Most general pharmaceutical formulation techniques can be found in Pharmaceutical Preformulation and Formulation (2nd Edition edited by Mark Gibson) and Pharmaceutical Excipients: Properties, Functionality and Applications in Research and Industry (edited by Otilia M Y Koo, published by Wiley).

Most general pharmacological techniques can be found in A Textbook of Clinical Pharmacology and Therapeutics (5th Edition published by Arnold Hodder). Most general techniques on the prescribing, dispensing, and administering of medicines can be found in the British National Formulary 72 (published jointly by BMJ Publishing Group Ltd and Royal Pharmaceutical Society).

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary 5 of many of the terms used in this disclosure. For chemical terms, the skilled person may refer to the International Union of Pure and Applied Chemistry (IUPAC).

Units, prefixes, and symbols are denoted in their Systéme International d'Unités (SI) accepted form. Numeric ranges are inclusive of the numbers defining the range.

Certain aspects of the invention provide a method of visualizing formation of a protein complex comprising C3b, and/or complement factor I (FI), and/or a complement factor I cofactor protein.

Certain aspects of the invention provide a method of visualizing formation of a protein complex comprising C3b, and complement factor I (FI), and/or either complement factor H (FH), factor H-like protein 1 (FHL-1), soluble CR1 (sCR1), or soluble membrane cofactor protein (sMCP).

Certain aspects of the invention provide a method of visualizing formation of a protein complex comprising variants of C3b, and complement factor I (FI), and/or either FH, FHL-1, sCR1, or sMCP.

Certain aspects of the invention provide a method for characterizing the formation of a protein complex comprising C3b, and FI and/or either FH, FHL-1, sCR1, or sMCP.

Certain aspects of the invention provide a method for characterizing the formation of a protein complex comprising variants of C3b, and FI and/or either FH, FHL-1, sCR1, or sMCP.

Certain aspects of the invention provide a method of characterizing a protein and/or drug molecule binding associated with a protein complex comprising C3b, and FI and/or an FI cofactor protein.

Certain aspects of the invention provide a method of characterizing a protein and/or drug molecule binding associated with a protein complex comprising C3b, and FI and/or either FH, FHL-1, sCR1, or sMCP.

Certain aspects of the invention provide a method of characterizing a protein and/or drug molecule binding associated with a protein complex comprising variants of C3b, and FI and/or either FH, FHL-1, sCR1, or sMCP.

Certain aspects of the invention provide a sensitive and reproducible assay for determining formation and/or one or more binding event of a protein complex comprising C3b, and FI and/or either FH, FHL-1, sCR1, or sMCP. Aptly, the assay may be able to determine formation of a protein complex comprising C3b, and FI and/or either FH, FHL-1, sCR1, or sMCP in real-time. Aptly, the assay may be able to assess one or more binding events associated with a protein complex comprising C3b, and FI and/or either FH, FHL-1, sCR1, or sMCP in real-time.

Certain aspects of the invention provide for a sensitive and reproducible assay for assessing formation and/or one or more binding event of a protein complex comprising variants of C3b, and/or FI and an FI cofactor protein, without the need to restore an immobilized substrate.

As used herein the term “binding event” may refer to the forming of a complex of any molecule or substance with a target molecule of interest (e.g. a protein).

Certain aspects of the invention provide a sensitive and reproducible assay for assessing formation and/or one or more binding events of a protein complex comprising variants of C3b, and FI and/or either FH, FHL-1, sCR1, or sMCP without the need to restore an immobilized substrate.

It is considered that the present inventors have devised a method for visualizing the formation of a protein complex comprising C3b, and FI and/or either FH, FHL-1, sCR1, or sMCP or a related complex. Aptly, the formation of the protein complex may be visualized in real time.

It is considered that the present inventors have devised a method for visualizing binding events associated with a protein complex comprising C3b, and FI and/or either FH, FHL-1, sCR1, or sMCP or a related complex during formation of said protein complex. Aptly, the visualization may occur in real time.

It is considered that the present inventors have devised a method for visualizing binding events associated with a protein complex comprising C3b, and FI and/or either FH, FHL-1, sCR1, or sMCP, or a related complex, following formation of the protein complex. Aptly, the visualization may occur in real time.

In certain embodiments, the protein complex is comprised of several components. The components may be selected from C3b, FI, and one or more of FH, FHL-1, sCR1, or sMCP. In other embodiments, the protein complex may comprise one or more components selected from C3b, FI, FH, FHL-1, sCR1 and sMCP.

In certain embodiments, the protein complex comprises one or more variant protein component. Aptly, the complex comprises a variant selected from one or more of C3b, FI, and either FH, FHL-1, sCR1, or sMCP. In other embodiments, the analyzed complex may comprise one or more of the variant protein components from variant C3b, FI, FH, FHL-1, sCR1, or sMCP.

In certain embodiments, the invention provides detection of binding events associated with the formation of a protein complex comprising C3b, and complement factor I (FI), and/or either FH, FHL-1, sCR1, or sMCP.

In certain embodiments, the invention provides detection of binding events associated with the formation of a protein complex comprising variants of C3b, and complement factor I (FI), and/or either FH, FHL-1, sCR1, or sMCP.

In certain embodiments, the invention provides detection of binding events of binding partners at a protein complex comprising C3b, and complement factor I (FI), and/or either FH, FHL-1, sCR1, or sMCP. For example, binding of a ligand.

In certain embodiments, the invention provides detection of binding events of binding partners at a protein complex comprising variants of C3b, and complement factor I (FI), and/or either FH, FHL-1, sCR1, or sMCP. For example, binding of a ligand.

In certain embodiments, the invention provides for analysis of binding interactions of a binding partner at a complex comprising one or more of the protein components from C3b, FI, FH, FHL-1, sCR1 or sMCP.

In certain embodiments, the invention provides for analysis of binding interactions of a binding partner at a complex comprising of one or more of the variant protein components from variant C3b, FI, FH, FHL-1, sCR1 or sMCP.

Certain aspects of the invention provide a method of visualizing formation of a protein complex comprising C4b, and complement factor I (FI), and/or a complement factor I cofactor protein.

Certain aspects of the invention provide a method of visualizing formation of a protein complex comprising C4b, and complement factor I (FI), and/or either sMCP, sCR1, or C4b binding protein (C4 bp)

Certain aspects of the invention provide a method of visualizing formation of a protein complex comprising variants of C4b, and complement factor I (FI), and/or either sMCP, sCR1 or C4 bp.

Certain aspects of the invention provide a method for characterizing the formation of a protein complex comprising C4b, and FI and/or either sMCP, sCR1, or C4 bp.

Certain aspects of the invention provide a method for characterizing the formation of a protein complex comprising variants of C4b, and FI and/or either sMCP, sCR1, or C4 bp.

Certain aspects of the invention provide a method of characterizing a protein and/or drug molecule binding associated with a protein complex comprising C4b, and FI and/or an FI cofactor protein.

Certain aspects of the invention provide a method of characterizing a protein and/or drug molecule binding associated with a protein complex comprising C4b, and FI and/or either sMCP, sCR1, or C4 bp.

Certain aspects of the invention provide a method of characterizing a protein and/or drug molecule binding associated with a protein complex comprising variants of C4b and FI and/or either sMCP, sCR1 or C4 bp.

Certain aspects of the invention provide a sensitive and reproducible assay for determining formation and/or one or more binding event of a protein complex comprising C4b, and FI and/or either sMCP, sCR1 or C4 bp. Aptly, the assay may be able to determine formation of a protein complex comprising C4b, and FI and/or either sMCP, sCR1 or C4 bp in real-time. Aptly, the assay may be able to assess one or more binding events associated with a protein complex comprising C4b, and FI and/or either sMCP, sCR1 or C4 bp in real-time.

Certain aspects of the invention provide for a sensitive and reproducible assay for assessing formation and/or one or more binding event of a protein complex comprising variants of C4b, and FI and/or an FI cofactor protein, without the need to restore an immobilized substrate.

As used herein the term “binding event” may refer to the forming of a complex of any molecule or substance with a target molecule of interest (e.g. a protein).

Certain aspects of the invention provide a sensitive and reproducible assay for assessing formation and/or one or more binding events of a protein complex comprising variants of C4b, and FI and/or either sMCP, sCR1 or C4 bp without the need to restore an immobilized substrate.

It is considered that the present inventors have devised a method for visualizing the formation of a protein complex comprising C4b, and FI and/or either sMCP, sCR1 or C4 bp or a related complex. Aptly, the formation of the protein complex may be visualized in real time.

It is considered that the present inventors have devised a method for visualizing binding events associated with a protein complex comprising C4b, and FI and/or either sMCP, sCR1 or C4 bp or a related complex during formation of said protein complex. Aptly, the visualization may occur in real time.

It is considered that the present inventors have devised a method for visualizing binding events associated with a protein complex comprising C4b, and FI and/or either sMCP, sCR1 or C4 bp or a related complex, following formation of the protein complex. Aptly, the visualization may occur in real time.

In certain embodiments, the protein complex is comprised of several components. The components may be selected from C4b, FI, and one or more of sMCP, sCR1 or C4 bp. In other embodiments, the protein complex may comprise one or more components selected from C4b, FI, sMCP, sCR1 or C4 bp.

In certain embodiments, the protein complex comprises one or more variant protein component. Aptly, the complex comprises a variant selected from one or more of C4b, FI, and sMCP, sCR1 or C4 bp. In other embodiments, the analyzed complex may comprise one or more of the variant protein components from variant C4b, FI, sMCP, sCR1 or C4 bp.

In certain embodiments, the invention provides detection of binding events associated with the formation of a protein complex comprising C4b, and complement factor I (FI), and/or either sMCP, sCR1 or C4 bp.

In certain embodiments, the invention provides detection of binding events associated with the formation of a protein complex comprising variants of C4b, and complement factor I (FI), and/or either sMCP, sCR1 or C4 bp.

In certain embodiments, the invention provides detection of binding events of binding partners at a protein complex comprising C4b, and complement factor I (FI), and/or either sMCP, sCR1 or C4 bp. For example, binding of a ligand.

In certain embodiments, the invention provides detection of binding events of binding partners at a protein complex comprising variants of C4b, and complement factor I (FI), and/or either sMCP, sCR1 or C4 bp. For example, binding of a ligand.

In certain embodiments, the invention provides for analysis of binding interactions of a binding partner at a complex comprising one or more of the protein components from C4b, FI, sCR1, sMCP or C4 bp.

In certain embodiments, the invention provides for analysis of binding interactions of a binding partner at a complex comprising of one or more of the variant protein components from variant C4b, FI, sMCP, sCR1 or C4 bp.

In certain embodiments, one or more of the components of the protein complex is a non-crystalline component. For example, the C3b or C4b e.g. variant C3b is a non-crystalline protein. In certain embodiments, more than one e.g. all of the components of the protein complex are non-crystalline. Thus, in certain embodiments, the invention provides a protein complex which comprises one or more amorphous proteins. In certain embodiments, the protein complex comprises an amorphous C3b protein or variant thereof, or C4b protein or variant thereof.

As used herein the term “complement factor I cofactor protein” refers to any protein with FI cofactor activity. In the context of the present invention, FI cofactor activity refers to any protein that facilitates or promotes binding of FI within or to a protein complex. Aptly, an FI cofactor protein may be a protein that facilitates or promotes binding of FI to a protein complex comprising C3b. In certain embodiments, FH, FHL-1, sCR1, or sMCP may be an FI cofactor protein. In certain embodiments, C4 bp may be an FI cofactor protein.

The protein C3b is cleaved from the soluble plasma protein C3. C3b participates in immune adherence reactions and enhances phagocytosis by attaching to target surfaces to amplify complement system response. C3b is a 177 kDa protein comprised of 12 distinct domains. It binds a vast array of proteins and receptors to affect its function. C3b is responsible for triggering a proteolytic cascade leading to the eventual cleavage of C5 to C5b, a key step towards the formation of the potentially cytolytic membrane-attack complex. Numerous human diseases are linked to inadequate regulation of C3b. A C3b polypeptide sequence is shown in SEQ. ID. No. 9.

In certain embodiments, the C3b protein or variant thereof is a mammalian C3b protein or variant thereof. In certain embodiments, the mammalian C3b or variant thereof is a mouse C3b protein or variant thereof. In certain embodiments, the mammalian C3b or variant thereof is a human C3b protein or variant thereof. In certain embodiments, the human C3b protein comprises an amino acid sequence selected from an amino acid sequence as set forth in SEQ ID NO: 9 or an amino acid sequence having at least 85% sequence identity to the amino acid sequence as set forth in SEQ ID NO: 9 In certain embodiments, the C3b protein may be comprised of a substantially truncated variant of the amino acid sequence as set forth in SEQ ID No 9, or a substantially truncated amino acid sequence having at least 85% sequence identity to the amino acid sequence as derived from the sequence as set forth in SEQ. ID. NO: 9. In certain embodiments, the human C3b protein comprises an amino acid sequence selected from an amino acid sequence as set forth in SEQ ID NO: 9 or an amino acid sequence having at least 85%, e.g. 86%, 87%, 88%, 89%, 90%, 91%, 92%, 93%, 94%, 95%, 96%, 97%, 98% or 99% sequence identity to the amino acid sequence as set forth in SEQ ID NO: 9.

In certain embodiments, the C4b protein or variant thereof is a mammalian C4b protein or variant thereof. In certain embodiments, the mammalian C4b or variant thereof is a mouse C4b protein or variant thereof. In certain embodiments, the mammalian C4b or variant thereof is a human C4b protein or variant thereof. In certain embodiments, the human C4b protein comprises an amino acid sequence selected from an amino acid sequence as set forth in SEQ ID NO: 18 or an amino acid sequence having at least 85% sequence identity to the amino acid sequence as set forth in SEQ ID NO: 18 In certain embodiments, the C4b protein may be comprised of a substantially truncated variant of the amino acid sequence as set forth in SEQ ID No 18, or a substantially truncated amino acid sequence having at least 85% sequence identity to the amino acid sequence as derived from the sequence as set forth in SEQ. ID. NO: 18. In certain embodiments, the human C4b protein comprises an amino acid sequence selected from an amino acid sequence as set forth in SEQ ID NO: 18 or an amino acid sequence having at least 85%, e.g. 86%, 87%, 88%, 89%, 90%, 91%, 92%, 93%, 94%, 95%, 96%, 97%, 98% or 99% sequence identity to the amino acid sequence as set forth in SEQ ID NO: 18.

As used herein, the term “protein” can be used interchangeably with “peptide” or “polypeptide”, means at least two covalently attached alpha amino acid residues linked by a peptidyl bond.

The term protein encompasses purified natural products, or chemical products, which may be produced partially or wholly using recombinant or synthetic techniques. The term protein may refer to a complex of more than one polypeptide, such as a dimer or other multimer, a fusion protein, a protein variant, or derivative thereof. The term also includes modified proteins, for example, a protein modified by glycosylation, acetylation, phosphorylation, pegylation, ubiquitination, and so forth. A protein may comprise amino acids not encoded by a nucleic acid codon.

The term “isolated” as used herein refers to a biological component (such as protein) that has been substantially separated or purified away from other biological components present in an isolated biological sample, and/or has been substantially separated or purified away from other biological components in the cell of the organism in which the component naturally occurs, i.e., other chromosomal and extra chromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins, and peptides.

The terms “recombinant” and “recombinant expression” are well-known in the art. The term “recombinant expression”, as used herein, relates to transcription and translation of an exogenous gene in a host organism. Exogenous DNA refers to any deoxyribonucleic acid that originates outside of the host cell. The exogenous DNA may be integrated in the genome of the host or expressed from a non-integrating element. A recombinant protein includes any polypeptide expressed or capable of being expressed from a recombinant nucleic acid.

In certain embodiments, the protein components described herein are expressed and purified from a recombinant system. In certain embodiments the protein components are derived from a recombinant expression system.

In certain embodiments, the invention is directed to proteins having modifications in their sequence. The terms “sequence identity”, “percent identity” and “sequence percent identity” in the context of two or more nucleic acids or polypeptides, refer to two or more sequences or sub-sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection.

Various algorithms and software are known in the art that can be used to obtain alignments of amino acid or nucleotide sequences. Suitable programs to determine percent sequence identity include for example the BLAST suite of programs available from the U.S. government's National Center for Biotechnology. Information BLAST web site (http://blast.ncbi.nlm.nih.gov/Blast.cgi). Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by alignment software. In certain embodiments, the default parameters of the alignment software.

The term “wild-type” may refer to a gene sequence, protein sequence, gene region sequence or genome region sequence which prevails among individuals of a particular population in natural conditions, as distinct from an atypical mutant type.

The term “variant” may refer to a protein sequence, gene sequence, gene region sequence, or genome region sequence which comprise an alteration from its respective wild-type sequence as a consequence of a genetic mutation(s).

Substitutional variants of proteins are those in which at least one amino acid residue in the amino acid sequence has been removed and a different amino acid residue inserted in its place. The term “conservative substitution” as used herein relates to the substitution of one or more amino acid residues for amino acid residues having similar biochemical properties.

Screening of variants of C3b, C4b, FI, C4 bp FH, FHL-1, sCR1, and/or sMCP as described herein can be used to identify which amino acid residues can tolerate an amino acid residue substitution. Protein variants may also refer to substantially truncated protein variants.

Complement Factor H is an abundant 155 kDa serum glycoprotein with 20 distinct modules, termed complement control protein modules (CCP). Factor H binds competitively with activating factors at C3b, thereby acting to suppress C3b convertase activity. A full length human FH polypeptide sequence is shown in SEQ ID NO: 1.

Once bound to C3b, Factor H recruits FI which then cleaves C3b to its inactive form, iC3b. Thus, FI is an important complement regulator. It is expressed in numerous tissues but principally by liver hepatocytes. FI is a heterodimer in which the two chains are linked together by a disulphide bond. The heavy chain contains the Factor I membrane attack complex (FIMAC) module, a CD5 domain and two low density lipoprotein receptor domains (LDLr). The light chain comprises a serine protease domain, the active site of which consists of a triad of His380, Asp439 and Ser525 (Precursor FI numbering). A wild-type FI heavy chain amino acid sequence is shown in SEQ ID NO: 4 and an FI light chain amino acid sequence is shown in SEQ ID NO: 5.

When FI is synthesized, it is initially made as a single chain precursor (precursor FI protein, or pro-FI), in which a four-residue linker peptide (RRKR) connects the heavy chain to the light chain (SEQ ID NO: 6). Furthermore, precursor FI also comprises a signal peptide (amino acids 1-18) which is typically cleaved before secretion from the cell. Thus, as used herein, the term “precursor FI protein” is used to refer to a single chain precursor complement Factor I protein which comprises a four-residue linker peptide (RRKR) and signal peptide. The precursor FI protein is substantially inactive and has essentially no C3, C3b-inactivating or iC3b-degradation activity. During processing to form mature FI, the precursor FI protein is cleaved by a calcium-dependent serine endoprotease, furin, leaving the heavy chain and light chain of full-length mature FI held together by a single disulphide bond. Reference to amino acid numbering of FI refers to amino acid numbering of the precursor FI prior to cleavage by a calcium-dependent serine endoprotease. WO2018/170152 A1 discloses a method for recombinant expression of pro-FI followed by in vitro incubation of purified recombinant pro-FI with Furin to produce mature FI.

As mentioned supra, Complement Factor H is comprised of 20 distinct CCP domains. The first four domains (CCP1-4) of FH are necessary and sufficient for regulation of complement in the fluid phase. Indeed, the FH N-terminus CCP1-4 domains mediate binding to C3b and cofactor activity for FI. Consequently, binding assays characterizing FH binding in vitro may utilise a truncated recombinant protein comprising CCPs 1-4. Thus, FH CCP 1-4 can be used in methods provided herein. In addition, the C-terminal CCPs 19-20 are also known to interact with C3b and partake in protein binding interactions. Consequently, recombinant proteins further comprising, or solely comprising, CCPs 19-20 may also be utilised and characterised in methods provided herein.

Indeed, in certain embodiments of the present invention, the FH protein or variant thereof is a mammalian FH protein or variant thereof. In certain embodiments, the mammalian FH or variant thereof is mouse FH protein or variant thereof. In certain embodiments, the mammalian FH or variant thereof is a human FH protein or variant thereof. In certain embodiments, the human FH protein comprises an amino acid sequence selected from an amino acid sequence as set forth in SEQ ID NO: 1 or an amino acid sequence having at least 85% sequence identity, e.g. 86%, 87%, 88%, 89%, 90%, 91%, 92%, 93%, 94%, 95%, 96%, 97%, 98% or 99% to the amino acid sequence as set forth in SEQ. ID. No. 1. In certain embodiments, the FH protein may be comprised of a substantially truncated variant of the amino acid sequence as set forth in SEQ ID NO: 1, or a substantially truncated amino acid sequence having at least 85% e.g. 86%, 87%, 88%, 89%, 90%, 91%, 92%, 93%, 94%, 95%, 96%, 97%, 98% or 99% sequence identity to a contiguous sequence comprised in the amino acid sequence as derived from the sequence as set forth in SEQ. ID. NO: 1.

In certain embodiments, a substantially truncated variant of FH protein may comprise CCP domains 1-4. In certain embodiments, a substantially truncated variant of FH protein may comprise CCP domains 1-4 and/or CCP domains 19 and 20. In certain embodiments, the substantially truncated FH variant may comprise an amino acid sequence as set forth in SEQ ID NO: 2, or a substantially truncated amino acid sequence having at least 85% e.g. 86%, 87%, 88%, 89%, 90%, 91%, 92%, 93%, 94%, 95%, 96%, 97%, 98% or 99% sequence identity to a contiguous sequence comprised in the he amino acid sequence as derived from the sequence as set forth in SEQ. ID. No. 2.

In certain embodiments, the FI protein or variant thereof is a mammalian FI protein or variant thereof. In certain embodiments, the mammalian FI or variant thereof is a mouse FI protein or variant thereof. In certain embodiments, the mammalian FI or variant thereof is a human FI protein or variant thereof. In certain embodiments, the human Complement Factor I protein comprises a first amino acid sequence selected from an amino acid sequence as set forth in SEQ. ID. No. 4 (heavy chain) or an amino acid sequence having at least 85% e.g. 86%, 87%, 88%, 89%, 90%, 91%, 92%, 93%, 94%, 95%, 96%, 97%, 98% or 99% sequence identity to the amino acid sequence as set forth in SEQ. ID. No. 4, and a second amino acid sequence as set forth in SEQ. ID. 5 (light chain) or an amino acid sequence having at least 85% e.g. 86%, 87%, 88%, 89%, 90%, 91%, 92%, 93%, 94%, 95%, 96%, 97%, 98% or 99% sequence identity to the amino acid sequence set forth in SEQ. ID. No. 5, wherein the first and second amino acid sequences are linked via a disulphide bond.

In certain embodiments, the FI protein may be comprised of substantially truncated variants of the amino acid sequences as set forth in SEQ. ID. No. 4 and SEQ. ID. No 5, or substantially truncated amino acid sequences having at least 85% e.g. 86%, 87%, 88%, 89%, 90%, 91%, 92%, 93%, 94%, 95%, 96%, 97%, 98% or 99% sequence identity to the amino acid sequence as derived from sequences as set forth in SEQ. ID. No. 4 and SEQ ID No. 5, respectively, wherein the first and second amino acid sequences are linked via a disulphide bond.

In certain embodiments, the FI component of the protein complex comprising C3b, and FI and/or either FH, FHL-1, sCR1, or sMCP, comprises a mutation at amino acid position 380, 429, and/or 525. In certain embodiments, the protein complex comprises a truncated FI component comprising an amino acid mutation wherein the mutation corresponds to amino acid position 380, 429, and/or 525 of full-length precursor FI. In embodiments, the FI component comprises an amino acid mutation corresponding to amino acid position 525 of full-length precursor FI. In further embodiments, the mutation is an S525A mutation (SEQ ID NO: 7). In alternative embodiments, the FI component comprises an amino acid mutation corresponding to amino acid position 380 of full-length precursor FI. In further embodiments, the mutation is an H380R mutation (SEQ ID NO: 16).

In certain embodiments, the FI component of the protein complex comprising C4b, and FI and/or either sMCP, sCR1, or C4 bp, comprises a mutation at amino acid position 380, 429, and/or 525. In certain embodiments, the protein complex comprises a truncated FI component comprising an amino acid mutation wherein the mutation corresponds to amino acid position 380, 429, and/or 525 of full-length precursor FI. In embodiments, the FI component comprises an amino acid mutation corresponding to amino acid position 525 of full-length precursor FI. In further embodiments, the mutation is an S525A mutation (SEQ ID NO: 7). In alternative embodiments, the FI component comprises an amino acid mutation corresponding to amino acid position 380 of full-length precursor FI. In certain further embodiments, the mutation is an H380R mutation (SEQ ID NO: 16).

Thus, in certain embodiments, the human Complement Factor I protein comprises a first amino acid sequence selected from an amino acid sequence as set forth in SEQ. ID. No. 4 (heavy chain) or an amino acid sequence having at least 85% sequence identity to the amino acid sequence as set forth in SEQ. ID. No. 4, and a second amino acid sequence as set forth in SEQ. ID. No. 8 (light chain) or SEQ ID No. 17 (light chain), or an amino acid sequence having at least 85% sequence identity to the amino acid sequence set forth in SEQ. ID. No. 8 or SEQ ID No. 17, wherein the first and second amino acid sequences are linked via a disulphide bond.

In alternative embodiments, FHL-1, sCR1 or sMCP can act as cofactors to recruit FI to the protein complex instead of FH.

The 42 kDa human factor-H-like protein 1 (FHL-1) is encoded by an mRNA which is derived from the CFHgene by means of alternative splicing. FH and FHL-1 are organized into a series of repetitive elements, termed short consensus repeats (SCRs). These SCRs broadly correspond to the above described CCPs. FHL-1 and FH are comprised of 7 and 20 consecutive SCRs, respectively. Both FHL-1 and FH possess cofactor activity in factor-I-mediated cleavage of C3b and decay-accelerating activity, as they support the dissociation of the C3bBb complex. Indeed, FHL-1 is an important regulator of the complement system by acting as a cofactor for the Factor I-mediated cleavage of soluble/bound C3b. Mutations in FHL-1 have been associated with susceptibility to complex diseases associated with aberrant complement activity.

In certain embodiments of the present invention, the FHL-1 protein or variant thereof is a mammalian FHL-1 protein or variant thereof. In certain embodiments, the mammalian FHL-1 or variant thereof is mouse FHL-1 protein or variant thereof. In certain embodiments, the mammalian FHL-1 or variant thereof is a human FHL-1 protein or variant thereof. In certain embodiments, the human FHL-1 protein comprises an amino acid sequence selected from an amino acid sequence as set forth in SEQ. ID. No:14 or an amino acid sequence having at least 85% e.g. 86%, 87%, 88%, 89%, 90%, 91%, 92%, 93%, 94%, 95%, 96%, 97%, 98% or 99% sequence identity to the amino acid sequence as set forth in SEQ. ID. No. 14. In certain embodiments, the FHL-1 protein comprises a substantially truncated variant amino acid sequence. In certain embodiments, the FHL-1 protein may be a substantially truncated variant of the amino acid sequence as set forth in SEQ ID No 14, or a substantially truncated amino acid sequence having at least 85% sequence identity to a contiguous amino acid sequence comprised in the amino acid sequence as derived from the sequence as set forth in SEQ. ID. No. 14.

In certain embodiments, FHL-1 protein may lack a signal peptide (amino acids 1-18). In certain embodiments, a substantially truncated FHL-1 variant may comprise an amino acid sequence as set forth in SEQ ID NO: 15, or a substantially truncated amino acid sequence having at least 85% e.g. 86%, 87%, 88%, 89%, 90%, 91%, 92%, 93%, 94%, 95%, 96%, 97%, 98% or 99% sequence identity to a contiguous amino acid sequence comprised in the amino acid sequence as derived from the sequence as set forth in SEQ. ID. No. 15.

The human complement receptor type 1 (CR1, C3b/C4b receptor, CD35) (SEQ ID NO: 10) is a polymorphic membrane glycoprotein expressed on human erythrocytes, peripheral leukocytes, plasma and renal glomerular podocytes, which consists of transmembrane and cytoplasmic domains. CR1 is comprised of 30 repeating homologous SCR protein domains. CR1 can serve several regulatory functions, including co-factor activity with FI. A soluble form of CR1, called sCR1, is a CR1 produced by cleaving the transmembrane domain at the C-terminus. Soluble CR1 is a powerful inhibitor of complement activation. sCR1 is also a powerful regulator of the complement system by acting as a cofactor for the Complement Factor I-mediated cleavage of soluble/bound C3b. Mutations in CR1 have been associated with susceptibility to complex diseases associated with aberrant complement activity.

In certain embodiments of the present invention, the sCR1 protein or variant thereof is a mammalian sCR1 protein or variant thereof. In certain embodiments, the mammalian sCR1 or variant thereof is a mouse sCR1 protein or variant thereof. In certain embodiments, the mammalian sCR1 or variant thereof is a human sCR1 protein or variant thereof. In certain embodiments, the human sCR1 protein comprises an amino acid sequence selected from an amino acid sequence as set forth in SEQ ID NO: 11 or an amino acid sequence having at least 85% e.g. 86%, 87%, 88%, 89%, 90%, 91%, 92%, 93%, 94%, 95%, 96%, 97%, 98% or 99% sequence identity to the amino acid sequence as set forth in SEQ ID NO: 11. In certain embodiments, the sCR1 protein comprises a substantially truncated variant amino acid sequence. In certain embodiments, the sCR1 protein may be comprised of a substantially truncated variant of the amino acid sequence as set forth in SEQ ID NO: 11, or a substantially truncated amino acid sequence having at least 85% e.g. 86%, 87%, 88%, 89%, 90%, 91%, 92%, 93%, 94%, 95%, 96%, 97%, 98% or 99% sequence identity to the amino acid sequence as derived from sequence as set forth in SEQ ID NO: 11.

Membrane cofactor protein (MCP) is a widely expressed glycoprotein that inhibits complement activation on host cells. MCP is a protein with two distinct C3b interaction domains, a transmembrane domain, and an intracellular cytoplasmic domain. Deficiencies of membrane cofactor protein (MCP) are associated with atypical HUS and other complement disorders. Indeed, MCP mutations account for approximately 10% of all atypical HUS. MCP is an important regulator of the complement system by acting as a cofactor for the Complement Factor I-mediated cleavage of soluble/bound C3b. Mutations in MCP have been associated with susceptibility to complex diseases associated with aberrant complement activity.

In certain embodiments the MCP protein is a soluble MCP protein (sMCP). In certain embodiments of the present invention, the MCP protein or variant thereof is a mammalian MCP protein or variant thereof. In certain embodiments, the mammalian MCP or variant thereof is a mouse MCP protein or variant thereof. In certain embodiments, the mammalian MCP or variant thereof is a human MCP protein or variant thereof. In certain embodiments, the human MCP protein comprises an amino acid sequence selected from an amino acid sequence as set forth in SEQ. ID. No. 12 or an amino acid sequence having at least 85% e.g. 86%, 87%, 88%, 89%, 90%, 91%, 92%, 93%, 94%, 95%, 96%, 97%, 98% or 99% sequence identity to the amino acid sequence as set forth in SEQ. ID. No. 12. In certain embodiments, the MCP protein comprises a substantially truncated variant amino acid sequences. In certain embodiments, the MCP protein may comprise a substantially truncated variant of a contiguous amino acid sequence comprised in the amino acid sequence as set forth in SEQ ID No 12, or a substantially truncated amino acid sequence having at least 85% e.g. 86%, 87%, 88%, 89%, 90%, 91%, 92%, 93%, 94%, 95%, 96%, 97%, 98% or 99% sequence identity to the amino acid sequence as derived from sequence as set forth in SEQ. ID. No. 12. For example, in certain embodiments, the human MCP protein comprises an amino acid sequence selected from an amino acid sequence as set forth in SEQ. ID. No. 13 or an amino acid sequence having at least 85% e.g. 86%, 87%, 88%, 89%, 90%, 91%, 92%, 93%, 94%, 95%, 96%, 97%, 98% or 99% sequence identity to the amino acid sequence as set forth in SEQ. ID. No. 13.

In certain embodiments of the present invention, the C4 bp protein or variant thereof is a mammalian C4 bp protein or variant thereof. In certain embodiments, the mammalian C4 bp or variant thereof is a mouse C4 bp protein or variant thereof. In certain embodiments, the mammalian C4 bp or variant thereof is a human C4 bp protein or variant thereof. In certain embodiments, the human C4 bp protein comprises an amino acid sequence selected from an amino acid sequence as set forth in SEQ. ID. No. 19 or an amino acid sequence having at least 85% (e.g. 86%, 87%, 88%, 89%, 90%, 91%, 92%, 93%, 94%, 95%, 96%, 97%, 98% or 99%) sequence identity to the amino acid sequence as set forth in SEQ. ID. No. 19 and an amino acid sequence selected from an amino acid sequence as set forth in SEQ. ID. No. 20 or an amino acid sequence having at least 85% sequence identity to the amino acid sequence as set forth in SEQ. ID. No. 20. In certain embodiments, the C4 bp protein comprises substantially truncated variant amino acid sequences. In certain embodiments, the C4 bp protein may comprise a substantially truncated variant of a contiguous amino acid sequence comprised in the amino acid sequence as set forth in SEQ ID. No. 19, or a substantially truncated amino acid sequence having at least 85% e.g. 86%, 87%, 88%, 89%, 90%, 91%, 92%, 93%, 94%, 95%, 96%, 97%, 98% or 99% sequence identity to the amino acid sequence as derived from sequence as set forth in SEQ. ID. No. 19. In certain embodiments, the C4 bp protein may comprise a substantially truncated variant of a contiguous amino acid sequence comprised in the amino acid sequence as set forth in SEQ ID No 20, or a substantially truncated amino acid sequence having at least 85% e.g. 86%, 87%, 88%, 89%, 90%, 91%, 92%, 93%, 94%, 95%, 96%, 97%, 98% or 99% sequence identity to the amino acid sequence as derived from sequence as set forth in SEQ. ID. No. 20

In certain embodiments, protein variants provided herein may have at least 85% sequence identity to an amino acid sequence described herein. For example, 86%, 87%, 88%, 89%, 90%, 91%, 92%, 93%, 94%, 95%, 96%, 97%, 98%, 99%, 99.5, or 99.9% sequence identity to an amino acid sequence recited herein.

In certain embodiments of the present invention, proteins described herein may further comprise an affinity purification tag. Conversely, in certain embodiments, the proteins described herein may not comprise an affinity purification tag.

In certain embodiments, the method relates to assessment of FH, or variant thereof, binding to C3b substrate, or variant thereof. In certain embodiments, the method relates to assessment of FI, or variant thereof, binding to C3b substrate, or variant thereof. In further embodiments, the method relates to assessment of FI binding to FH-C3b complex. In other embodiments the method relates to assessment of FI binding to FHL-1-C3b complex, sCR1-C3b complex, or sMCP-C3b complex. In other embodiments, the method relates to assessment of FH binding to FI-C3b complex. In other embodiments, the method relates to assessment of FHL-1, sCR1 or sMCP binding to FI-C3b complex.

In certain embodiments, the method relates to assessment of FH, or variant thereof, binding to C4b substrate, or variant thereof. In certain embodiments, the method relates to assessment of FI, or variant thereof, binding to C4b substrate, or variant thereof. In further embodiments, the method relates to assessment of FI binding to C4 bp-C4b complex. In other embodiments the method relates to assessment of FI binding to sCR1-C4b complex. In other embodiments the method relates to assessment of FI binding to sMCP-C4b complex. In other embodiments, the method relates to assessment of sCR1 binding to FI-C4b complex. In other embodiments, the method relates to assessment of C4 bp binding to FI-C4b complex. In other embodiments, the method relates to assessment of sMCP binding to FI-C4b complex.

A recurrent issue with the development of complement binding assays is the proteolytic destruction of the C3b substrate or C4b substrate upon binding of Complement factor I and Complement factor H. This is of particular issue if the binding assay requires immobilization of C3b or C4b to a surface, as is the case for Surface Plasmon Resonance based assays, for example. Proteolytic destruction of the surface immobilized substrate, for example C3b, necessitates restoration of the immobilized substrate on the surface.

Thus, in certain embodiments, the protein complex comprising C3b or C4b, and/or complement factor I (FI) and/or either complement factor H (FH), FHL-1, soluble CR1 (sCR1), C4 bp, or sMCP does not possess proteolytic activity. For example, a protein complex comprising C3b, and/or FI and/or either FH, FHL-1, sCR1, or sMCP, inactivates C3b by proteolytically cleaving the substrate to its inactive form—iC3b. The FI component of the protein complex performs the cleavage reaction. Mutations of the FI sequence may inactivate FI proteolytic activity. For example, one or more substitution at precursor FI amino acid positions 380, 429, and/or 525 are known to result in inactivation of FI proteolytic activity. Thus, in certain embodiments of the present invention, the FI component of the protein complex comprising C3b or C4b, and FI and/or either C4 bp FH, FHL-1, sCR1, or sMCP, comprises a mutation at amino acid position 380, 429, and/or 525. In certain embodiments, the FI comprises an amino acid mutation at the amino acid position corresponding to amino acid position 525 of full-length precursor FI. In further preferred embodiments, the mutation is an S525A mutation. In alternative embodiments, the FI comprises an amino acid mutation at the amino acid position corresponding to amino acid position H380 of full-length precursor FI e.g., H380R. The FI may be mammalian e.g. human FI.

In certain embodiments, the Complement Factor I protein or variant thereof comprises at least one mutation that results in an inactivation or reduction of Complement Factor I protease activity. In certain embodiments, the mutation is within the proteins active site.

The term “active site” may refer to any residues essential for, and/or involved in the catalytic activity of an enzyme (e.g. FI mediated proteolysis of C3b or C4b). The active site may include, but is not limited to, amino acid residues that directly partake in substrate binding and/or catalysis. As such, active site residues can be easily and readily identified through numerous methods known to the artisan.

In certain embodiments, the FI component of the protein complex comprising C3b, and/or FI and/or either FH, FHL-1, sCR1, or sMCP, comprises a mutation at amino acid position 380, 429, and/or 525. In certain embodiments, the protein complex comprises a truncated FI component comprising an amino acid mutation at amino acid position 380, 429, and/or 525 of full-length precursor FI. In embodiments, the FI component comprises an amino acid mutation corresponding to amino acid position 525 of full-length precursor FI. In further embodiments, the mutation is an S525A mutation. In other embodiments, the mutation is an H380R mutation. In certain embodiments, the FI component of the protein complex comprising C4b, and FI and/or either sCR1, or sMCP, or C4 bp, comprises a mutation at amino acid position 380, 429, and/or 525. In certain embodiments, the protein complex comprises a truncated FI component comprising an amino acid mutation at amino acid position 380, 429, and/or 525 of full-length precursor FI. In embodiments, the FI component comprises an amino acid mutation corresponding to amino acid position 525 of full-length precursor FI. In further embodiments, the mutation is an S525A mutation. In alternative embodiments, the FI component comprises an amino acid mutation corresponding to amino acid position 380 of full-length precursor FI. In embodiments, the mutation is an H380R mutation.

In certain embodiments, the polypeptide sequences of protein components comprising the protein complex comprising C3b, and FI and/or either FH, FHL-1, sCR1, or sMCP, may be the same as proteins expressed by a subject. In certain embodiments the subject may suffer from a complement mediated disorder. In certain embodiments, the complement system mediated disorder is atypical haemolytic uremic syndrome, microangiopathic hemolytic anaemia, age-related macular degeneration, C3 glomerulopathy, Alzheimer's disease and/or thrombocytopenia.

In certain embodiments, the polypeptide sequences of protein components comprising the protein complex comprising C4b, and FI and/or either sCR1, or sMCP, or C4 bp, may be the same as proteins expressed by a subject. In certain embodiments the subject may suffer from a complement mediated disorder. In certain embodiments, the complement system mediated disorder is atypical haemolytic uremic syndrome, microangiopathic hemolytic anaemia, age-related macular degeneration, C3 glomerulopathy, Alzheimer's disease and/or thrombocytopenia.

Mutations introduced to components of the method provided herein, wherein the mutation is introduced with the known purpose of inactivating proteolytic activity of a protein complex described herein (i.e. the AP regulatory TMC), are not considered to be corresponding to a subject, insofar as the purpose of the mutation is to facilitate the correct functioning of the method provided herein, and not to correspond to a mutation present in a subject.

In certain embodiments, protein or nucleic acid components of the method provided herein comprise the same sequence as those expressed within a subject. In further embodiments, the subject suffers from a complement system mediated disorder. In certain embodiments, the complement system mediated disorder is atypical haemolytic uremic syndrome, microangiopathic hemolytic anaemia, age-related macular degeneration, C3 glomerulopathy, Alzheimer's disease and/or thrombocytopenia. A comprehensive database of genetic variants of genes encoding complement proteins identified in diseases of complement can be found at the ‘Database of complement gene variants’ (www.complement-db.org)

Aptly, in certain embodiments, the invention provides a method in which the components of the protein complex comprising C3b, and/or FI and/or either FH, FHL-1, sCR1, or sMCP, correspond to genetic variants of a subject. Thus, certain embodiments of the invention relate to the generation and analysis of a protein complexes comprising C3b, and/or FI and/or either FH, FHL-1, sCR1, or sMCP, where in the protein components correspond to the genotype of a subject. In certain embodiments, the subject possesses genetic variants in CD46 (MCP), CD35 (CR1), CFH (which translates to FH and the alternatively spliced molecule FHL-1), CFI and/or C3 (C3b). Aptly, such genetic variants are associated with, or identified in a patient with, a complement system mediated disorder.

Aptly, in certain embodiments, the invention provides a method in which the components of the protein complex comprising C4b, and/or FI and/or either sMCP, sCR1 or C4 bp, correspond to genetic variants of a subject. Thus, certain embodiments of the invention relate to the generation and analysis of a protein complexes comprising C4b, and/or FI and/or either sMCP, sCR1 or C4 bp where in the protein components correspond to the genotype of a subject. In certain embodiments, the subject possesses genetic variants in C4BPA (C4 bp alpha chain), C4BPB (C4 bp beta chain), CD35 (CR1), CD46 (MCP) CFI and/or C4B (C4b). Aptly, such genetic variants are associated with, or identified in a patient with, a complement system mediated disorder.

The term “binding partner” and “ligand” are herein used interchangeably. The terms refer to any molecule or substance that forms a complex with a target protein. A “ligand” or “binding partner” may be any chemical, protein or substance, or composition thereof that binds to a target protein. Thus, the “ligand” or “binding partner” may be a protein, polypeptide, peptide, RNA, or DNA molecule. In a certain embodiment, the “ligand” or “binding partner” may be a pharmaceutical product, a cell metabolite, or a hormone e.g. in serum. In certain embodiments, a drug molecule may be naturally occurring or may be synthetically or recombinantly produced.

Certain embodiments of the invention assess the binding and function of biomolecules or drug molecules. The term “drug molecule” may refer to any chemical, protein or substance, or composition thereof that, upon introduction to a system, elicits a biochemical and/or physiological effect. Thus, the drug molecule may be a protein, polypeptide, peptide, RNA, or DNA molecule. In certain embodiments, the molecule may be a pharmaceutical product, a cell metabolite, or a hormone e.g. in serum. The drug molecule may be naturally occurring or may be synthetically or recombinantly produced.

The drug molecule may or may not bind to the target protein; in one aspect the method of the invention determines or assesses whether a particular molecule or compound is capable of binding to the target protein i.e. whether a drug molecule is a ligand (i.e. a candidate ligand molecule). Thus, the methods described herein can be used to screen a drug library for molecules which are capable of binding to the target protein. Some of the molecules tested may not bind, whereas others may bind to the target protein. Additionally, the methods described herein can be used to identify variants of small molecules known to bind to the target protein, which can bind the target protein with higher affinity (or alternatively with lower affinity). Thus, drug molecules can be mutated ligands or known (or unknown) target protein binding partners. Certain embodiments of the invention provide a method for identifying a candidate molecule for the treatment of an individual with a Complement mediated disorder.

Certain embodiments of the invention may be used to stratify a group of patients. The term “stratification” may refer to the identification of a group of patients with shared “biological” characteristics by using molecular, biochemical and imaging diagnostic testing to select the optimal management for the patients and achieve the best possible outcome in terms of (based on the category and disease characteristics): i) risk assessment and prevention, ii) achievement of the optimal treatment outcome, and/or iii) results of a clinical trial.

In certain embodiments, the protein or nucleic acid components comprise essentially the same sequence as those within a subject. In other embodiments, protein or nucleic acid components of the method provided herein comprise the same sequence as those within a subject. Thus, certain embodiments of the invention contemplate use as a tool for identifying candidate treatments on an individualized basis.

Therefore, certain embodiments comprise a method for evaluating the genetic profile of a subject. Genetic profiling can select a candidate treatment for a subject that could favorably change the clinical course for an individual with a condition or disease, such as a complement mediated disorder, for example, age-related macular degeneration. Genetic profiling may provide clinical benefit for individuals, such as identifying drug target(s) that provide favorable clinical outcome. Certain embodiments may be directed to evaluating the genetic profile of complement system mediated disorders on an individual basis. Certain embodiments provide a method for generating a genetic profile of a subject's complement system.

Genetic profiling can be performed by any known means for detecting a nucleic acid sequence in a biological sample. Determining the genetic profile can be achieved by methods that include but are not limited to, nucleic acid sequencing, such as a DNA sequencing or mRNA sequencing; in situ hybridization (ISH); fluorescent in situ hybridization (FISH); various types of microarray (mRNA expression arrays, protein arrays, etc); various types of sequencing (Sanger, pyrosequencing, etc); comparative genomic hybridization (CGH); NextGen sequencing; and any other appropriate technique to assay the presence or quantity of a nucleic acid molecule of interest. In various embodiments, any one or more of these methods can be used concurrently or consecutively for assessing a target gene described herein.

Genetic profiling of individual samples may be used to select one or more candidate treatments for a disorder in a subject, e.g., by identifying targets for drugs that may be effective for a given complement mediated disorder. For example, the candidate treatment can be a treatment known to have an effect on cells that differentially express genes as identified by molecular profiling techniques, which may have been studied and approved for a particular indication that is the same as or different from the indication of the subject from whom a biological sample is obtained and profiled.

Certain embodiments comprise assessment of known and novel biomarkers associated with C3b, C4b, complement factor I (FI), complement factor H (FH), FHL-1, soluble CR1 (sCR1), C4 bp and/or sMCP. A biomarker is a biological characteristic, which can be molecular, anatomic, physiologic, or biochemical. These characteristics can be measured and evaluated objectively. They may act as indicators of a normal or a pathogenic biological process. They may allow assessing the pharmacological response to a therapeutic intervention. A biomarker shows a specific physical trait or a measurable biologically produced change in the body that is linked to a disease or a particular health condition. A biomarker may be used to assess or detect: i) a specific disease as early as possible—diagnostic biomarker; ii) the risk of developing a disease—susceptibility/risk biomarker; iii) the evolution of a disease (indolent vs. aggressive)—prognostic biomarker-but it can be predictive too; vi) the response and the toxicity to a given treatment—predictive biomarker.

Thus, certain embodiments may be used to diagnose the effect of de novo mutations identified in CD46 (MCP), CD35 (CR1), CFH (which translates to FH and the alternatively spliced molecule FHL-1), C4B (C4b) C4BPA (C4 bp alpha chain), C4BPB (C4 bp beta chain), CFI and/or C3 (C3b). Certain embodiments comprise assessment of novel de novo mutations identified in a subject.

Aptly, certain embodiments comprise assessing one or more biomarkers, e.g. those identified in a subject.

In certain embodiments, the method is performed on an analytical platform e.g. a biophysical analysis platform. The term “biophysical analysis platform” refers to any analytical platform that can be used to assess the binding of molecules. Such analytical platforms may utilize physical properties of the analyte, substrate, or other reaction component to detect and quantify binding events in a given environment. Thus, a biophysical analysis platform typically comprises a sensor configured to detect the physical properties of the analyte, substrate, or other reaction component to be measured. Such platforms facilitate the characterisation of, for example, binding affinity, binding avidity, binding kinetics and/or binding thermodynamics. Such platforms are well known in the art and are widely used by the artisan. Such platforms, for example, include platforms capable of performing analysis of molecule binding based upon utilization of methods such as isothermal titration calorimetry (ITC), surface plasmon resonance (SPR), circular dichroism spectroscopy (CDS), microscale thermophoresis (MST), biolayer interferometry (BLI), an enzyme-linked immunosorbent assay (ELISA), Forster Resonance Energy Transfer assay, or an electrophoretic mobility shift assay (EMSA).

In certain embodiments, the invention involves the use of a biophysical analysis platform. Thus, in certain embodiments, a sensor configured to detect a signal created by a binding event is used. Aptly, the sensor may detect the signal created by the binding event over time to provide temporal resolution of the binding event(s). Thus, certain embodiments may provide for detection and characterisation of binding events in “real-time”. Embodiments may therefore provides for characterisation of, for example, binding kinetics and/or binding thermodynamics. Embodiments may characterise binding affinity and/or binding avidity. Techniques for measuring and evaluating binding affinity/avidity can be found, for example, at Jarmoskaite et al, eLife 2020; 9:e57264.

In certain embodiments, the biophysical analysis platform, is an in-silico molecular modelling platform. In-silico molecular modelling platforms refers to computer-assisted identification and characterization of binding events between binding partners (for example, a protein and ligand). Commonly, this is conducted through molecular dynamics simulations. Molecular dynamic simulations refer to computer-based molecular simulation methods in which the time evolution of interacting atoms, groups of atoms or molecules, including macromolecules, is followed by integrating their equations of motion. The atoms or molecules are allowed to interact for a period of time, giving a view of the motion of the atoms or molecules.

Molecular dynamic simulations can be physics-based simulations, energy-based simulations, or combinations thereof. Typically, the trajectories of atoms and molecules are determined by numerically solving the Newton's equations of motion for a system of interacting particles, where forces between the particles and potential energy are defined by molecular mechanics force fields. However, molecular dynamics simulations incorporating principles of quantum mechanics and hybrid classical-quantum mechanics simulations are also available and are contemplated herein. Energy based molecular dynamics simulation can calculate forces exerted by and among the members of a simulated system (e.g., atoms, groups of atoms, or molecules), including, but not limited to, the function of the distance, properties (e.g., charge, polarizability, etc.), and relation (e.g., bound or unbound) of the members of a system. Thus, molecular dynamics simulations can comprise steps of simulating a conformational change of all or part of a starting conformation of a molecule towards a different conformation of the molecule. Such changes can arise from changes to the positions of atoms or groups of atoms from their respective positions in a starting molecular structure of a molecule to their respective positions at the end of the simulation. Molecular dynamics simulation can also be used to simulate a conformational change of all or part of a starting conformation of a complex, for example a complex between a target protein and a compound, towards a different conformation of the complex. Therefore, it is understood that molecular dynamics simulation can be performed on the 3D structure of a target protein in the presence or absence of a compound that binds the target protein.

Molecular simulations can be used to monitor time-dependent processes of molecules, in order to study their structural, dynamic, and thermodynamic properties by solving the equation of motion, e.g., the chemical bonds within proteins may be allowed to flex, rotate, bend, or vibrate as allowed by the laws of chemistry and physics. This equation of motion provides information about the time dependence and magnitude of fluctuations in both positions and velocities of the given molecule. Interactions such as electrostatic forces, hydrophobic forces, van der Waals interactions, interactions with solvent and others may also be modeled in molecular dynamics simulations. A comprehensive understanding of In-silico molecular modelling techniques will be commonly known by the person skilled in the art. Information in respect of such methods and related methods can be found in “Adcock and McCammon, Chemical Reviews, 2006, 106, 5, 1589-1615”

In certain embodiments, the biophysical analysis platform is an isothermal titration calorimetry (ITC) platform. Principally, ITC is used to measure the equilibrium heat of binding of molecules (for example, a ligand to a protein, or a protein to a protein). ITC is used frequently in the characterization of the thermodynamics of molecular interactions. ITC is typically performed with an isothermal titration calorimeter. The instrument generally consists of two cells; a main cell for the macromolecule of concern and a reference cell, which is meant for the solvent. Both cells are kept at steady temperature and pressure. When a test molecule is titrated into the main cell, the instrument measures the heat change in the main cell, which is evolved or absorbed as a result of the binding of the newly introduced molecule to the main cell. From results of multiple-injection experiments, properties, such as, the association constant, the enthalpy, the Gibbs energy, and entropy changes, and the stoichiometry of binding, may be determined for a particular pairing between the test substance and test molecule. A comprehensive understanding of ITC based techniques will be known by the person skilled in the art. Information in respect of related methods can be found in “Leavitt & Freire, 2001, Current Opinion in Structural Biology, 11:560-6

In certain embodiments, the biophysical analysis platform is a circular dichroism spectroscopy-based platform. Circular dichroism (CD) is the differential absorption of left and right circularly polarized light by a solution containing molecules of interest. Typically, a signal is measured for chiral molecules, such as proteins, for example. A CD spectrum affords information about the bonds and structures responsible for this chirality. When a small binding partner (for example, a ligand) binds to a protein, it acquires an induced CD (ICD) spectrum through chiral perturbation to its structure or rearrangement of electrons. The wavelengths of this ICD are determined by the ligand's own absorption spectrum, and the intensity of the ICD spectrum is determined by the strength and geometry of its interaction with the protein. Consequently, ICD can be used to probe the binding of ligands to proteins. A comprehensive understanding of CD and ICD based techniques will be known by the person skilled in the art. Information in respect of related methods can be found in ‘Rodger et al (2005), Circular Dichroism Spectroscopy for the Study of Protein-Ligand Interactions. In: Ulrich Nienhaus G. (eds) Protein-Ligand Interactions. Methods in Molecular Biology™, vol 305. Humana Press’.

In certain embodiments, the biophysical analysis platform is a microscale thermophoresis (MST) platform. MST is based on the property of thermophoresis, the directed movement of molecules in a temperature gradient. Thermophoresis strongly depends on a number of molecular properties, including size, charge, hydration shell, and/or structural conformation. Thus, the technique is highly sensitive to virtually any change in molecular properties, allowing for a precise quantification of molecular binding events, irrelevant of sample size. During an MST experiment, a temperature gradient is generated within a sample by an infrared laser. The movement of molecules through the temperature gradient during and after laser excitation is detected and quantified using intrinsic fluorophores or covalently attached fluorophores joined to a target molecule within the sample. Differing thermophoresis among samples/conditions is then used to determine and quantify binding events. A comprehensive understanding of MST based techniques will be known by the person skilled in the art. Information in respect of related methods can be found in ‘Jerabek-Willemsen M, et al. Molecular interaction studies using microscale thermophoresis. Assay and Drug Development Technologies, 2011; 9(4):342-353.’

In certain embodiments, the biophysical analysis platform is an immunodetection platform. Immunodetection methods can be used for binding, purifying, removing, quantifying, and/or detecting biological components such as protein(s), polypeptide(s), peptide(s), hormone(s) and small molecule(s). Some immunodetection methods include enzyme linked immunosorbent assay (ELISA), radioimmunoassay (RIA), immunoradiometric assay, fluoroimmunoassay, chemiluminescent assay, bioluminescent assay, and Western blot, although this list is not exhaustive. Various useful immunodetection methods and their application have been described in the scientific literature and are well known in the art, such as, for example, e.g., Doolittle M H and Ben-Zeev O, Methods in Molecular Biology, 1999; 109:215-37; Gulbis B and Galand P, Human Pathology. 1993 December; 24(12):1271-85; and De Jager R et al., Seminars in Nuclear Medicine, 1993 April; 23(2):165-79. Such techniques include binding assays such as the various types of enzyme linked immunosorbent assays (ELISAs) and/or radioimmunoassays (RIA) are well known in the art, and will be known by the person skilled in the art.

In certain embodiments, the biophysical analysis platform is an electrophoretic mobility shift assay platform. The EMSA is used to detect proteins complexed with a radiolabeled substrate. EMSA underlies a wide range of qualitative and quantitative analyses for the characterization of biomolecule interaction systems. In a classical EMSA assay, solutions of protein and radiolabeled substrate are combined, and the resulting mixtures are subjected to electrophoresis under native conditions through polyacrylamide or agarose gel. After electrophoresis, the distribution of species containing radiolabeled ligand is determined, usually by autoradiography. In general, protein-substrate complexes migrate more slowly than the corresponding free substrate. Thus, binding events can be determined and quantified. A comprehensive understanding of EMSA will be known by the person skilled in the art. Information in respect of related methods can be found in Carrie Rocca et al, Evaluation of electrophoretic mobility shift assay as a method to determine plasma protein binding of siRNA, Bioanalysis, 10.4155/bio-2019-0151, 11, 21, (1927-1939), (2019) and Seo M et al, Label-Free Electrophoretic Mobility Shift Assay (EMSA) for Measuring Dissociation Constants of Protein-RNA Complexes. Current Protocol Nucleic Acid Chemistry. 2019; 76 (1):e70. doi:10.1002/cpnc.70.

In certain embodiments, the biophysical analysis platform is a Forster Resonance Energy Transfer platform. FRET is a fluorescence technique used to study molecular interactions of cells. FRET utilizes the non-radiative (dipole-dipole) energy transfer from a fluorescent donor to an acceptor that can take place only when the two fluorophores are situated at distances <˜10 nm. In the case of two proteins, for example, labelled with donor and acceptor tags, FRET occurs only when the two proteins interact with each other. FRET has therefore been widely exploited to study protein interactions. Methods for studying molecular interactions of protein complexes, and their ligands. A comprehensive understanding of FRET based techniques will be known by the person skilled in the art. Information in respect of related methods can be found in ‘FRET—Forster Resonance Energy Transfer: From Theory to Applications, Igor L. Medintz & Niko Hildebrandt, John Wiley & Sons, 2013.’

In certain embodiments, the biophysical analysis platform is a biolayer interferometry (BLI) platform. Bio-Layer Interferometry (BLI) is a label-free technology for measuring biomolecular interactions. It is an optical analytical technique that analyzes the interference pattern of white light reflected from two surfaces: a layer of immobilized protein on a biosensor tip, and an internal reference layer. Any change in the number or nature of molecules bound to the biosensor tip causes a shift in the interference pattern that can be measured in real-time. The binding between a ligand immobilized on the biosensor tip surface and an analyte in solution produces an increase in optical thickness at the biosensor tip, which results in a wavelength shift, which is a direct measure of the change in thickness of the biological layer. Interactions are measured in real time, providing the ability to monitor binding specificity, rates of association and dissociation, or concentration of biomolecules. A comprehensive understanding of BLI based techniques will be known by the person skilled in the art. Information in respect of related methods can be found in Bhagwat S, Kumar A (2018), Biolayer Interferometry and its Applications; Journal of Molecular Biology and Techniques 2(1):106.

In certain embodiments, the biophysical analysis platform is an analytical ultracentrifugation (AUC) platform. Analytical centrifugation is a technique for studying the hydrodynamic properties of a molecule as it moves through a fluid medium under the influence of an applied gravitational force. The hydrodynamic behavior of the sample in question depends on mass, density, and shape, so one can study these properties of a molecule by accelerating a sample preparation in a centrifugal field. The analytical ultracentrifuge (AUC) contains a built-in optical system allowing one to observe the movement of a sample as it is spun in a centrifuge rotor. The centrifuge permits molecules to be studied in their native state, in solution, and has been useful in characterizing how proteins and other biological macromolecules bind to one another to form higher ordered structures. The apparatus and theoretical background of AUC are described in the literature (see e.g., Peter Schuck., Biophysics Reviews (2013) 5:159-171)

In preferred embodiments, the biophysical analysis platform is a surface plasmon resonance (SPR) platform. A representative SPR based biophysical platform is the Biacore® instrumentation sold by GE Healthcare Life Sciences or Cytivia, which uses SPR for detecting interactions between molecules in a sample and molecular structures immobilized on a sensing surface. By using a Biacore® system it is possible to determine in real time without the use of labeling not only the presence and concentration of a particular molecule in a sample, but also additional interaction parameters such as, for instance, the association rate and dissociation rate constants for the molecular interaction. The apparatus and theoretical background are described in the literature (see e.g., Jonsson, U., et al, BioTechniques 11: 620-627 (1991)). Normally, the technique involves the immobilization of a ligand to the special optical sensor surface of a sensor chip (flow cell), contacting the sensor chip with a flow of sample containing the analyte of interest, and then measuring the change in the surface optical characteristics of the sensor chip arising from the binding between the ligand and the analyte. Further details on SPR are referenced in U.S. Pat. Nos. 5,313,264, 5,573,956 and 5,641,640.

Immobilisation of a protein complex on a surface can either be direct, by covalent coupling, or indirect, through capture by a covalently coupled molecule. For covalent coupling there are three main types of coupling chemistry, which utilize, respectively, amine (e.g. lysine), thiol (cysteine) or aldehyde (carbohydrate) functional groups on glycoproteins. Amine coupling exploits primary amine groups on the ligand after activation of the surface with 1-ethyl-3-(3-dimethylaminopropyl)-carbodiimide (EDC) and N-hydroxysuccinimide (NHS). Thiol coupling exploits thiol-disulfide exchange between thiol groups and active disulfides introduced on either the ligand or the surface matrix. In the case of C3b, thioester coupling refers specifically to nucleophilic attack of an internal thioester bond within C3 after cleavage by an AP C3 convertase already bound to the flow cell surface, a process which is described herein. Aldehyde coupling uses the reaction between hydrazine or carbohydrazide groups introduced on the surface and aldehyde groups obtained by oxidation of carbohydrates in the ligand.

In certain embodiments, protein complexes described herein are bound to a surface amine (e.g. lysine), thiol (cysteine) or aldehyde (carbohydrate) coupling.

All covalent coupling methods utilize free carboxymethyl groups on the sensor chip surface. They can therefore be used for any sensor chips that have carboxymethyl groups. If the protein to be immobilised has a surface-exposed disulphide or a free cysteine, ligand-thiol coupling is probably the method of choice. Failing this, amine coupling should be tried in the first instance. If amine coupling inactivates the protein (as assessed by ligand and/or mAb binding), aldehyde coupling can be attempted, provided that the protein is glycosylated. Detailed protocols are available from BIAcore for all coupling techniques.

In certain embodiments of the present invention, protein complexes described herein are bound to a surface amine (e.g. lysine), thiol (cysteine) or aldehyde (carbohydrate) coupling. In preferred embodiments, protein complexes described herein are bound to a surface through amine and/or thiol coupling and/or coupling via a thioester.

In certain embodiments, the C3b protein component or C4b protein component of the protein complex is bound to the surface.

In order to couple C3b to the chip surface via its thioester domain, after immobilisation of a small amount of C3b by amine coupling Complement Factor B and Factor D are used to build the AP C3 convertase (C3bBb). Subsequently, C3 was injected immediately across the convertase so that the C3 is cleaved to C3b by the chip-bound convertase. Rapid nucleophilic attack on the internal thioester of C3b results in covalent binding of C3b to the surface through an ester bond. Several subsequent cycles of convertase formation and C3 cleavage result in C3b covalently immobilised on the chip surface in a physiologically relevant way—coupling via the thioester results in all the C3b molecules oriented so that they are able to bind FH1-4 and subsequently FI.

In certain embodiments, the FI cofactor protein component of the protein complex is bound to the surface.

In certain embodiments the FI component of the protein complex is bound to the surface.

In certain embodiments, the invention provides a method for determining the binding interactions of any protein complex described herein. The term “binding interactions” refers to any protein-protein interaction, protein-ligand interaction, or ligand-ligand interaction occurring wherein the binding partners are labelled, tagged, immobilized and/or in solution that results in an association in which the respective molecules are in proximity to each other. Binding reactions can occur homogeneously or heterogeneously. Binding interactions may be irreversible or reversible. Binding interactions may be non-covalent (e.g. electrostatic interactions, ionic bonding, hydrogen bonding, halogen bonding, dipole-dipole interactions, dipole induced dipole interactions, Van der Waals forces, π-interactions, hydrophobic interaction), or covalent (e.g. single bond, double bond, triple bond).

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive.

The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

### Methods

Unless otherwise stated, general chemical reagents used in these methodologies were purchased from Sigma-Aldrich and Merck (now MilliporeSigma) (Dorset, UK) or ThermoFisher Scientific (Loughborough, UK). All products were analytical grade or equivalent. All Eppendorfs, falcon tubes, flasks and plates were produced by Corning (Corning, New York, USA) or Grenier Bio-One (Kremsmunster, Austria).

### Example 1—Immobilisation of C3b onto a Sensor Chip CM5 Using a BIAcore S200

Using the BIAcore S200 (General Electric (GE), Boston, MA, USA) the surface of a Carboxymethyl 5 (CM5) Sensor Chip (GE) was activated for amine coupling, by flowing N-(3-dimethylaminopropyl)-N′-ethylcarbodimide hydrochloride (EDC) and N-hydroxysuccinimide (NHS) over the surface of the selected flow cell on the chip. This process created a negatively charged chip surface, in order that protein to be immobilised (e.g. C3b) added in a low pH buffer was bound covalently, subsequently allowing analysis of interactions between the chip-bound protein and additional protein analytes flown across the chip, as measured by surface plasmon resonance (SPR). Initially, 800 resonance units (RU) of C3b were immobilised onto the chip, for an expected maximum binding of 100RU of FH1-4 analyte. This was deemed acceptable since the 800RU: 100RU ratio is significantly larger than the molecular weight ratio of ˜6:1 between chip bound ligand and analyte and therefore, binding sites on the chip were never saturated. C3b could be immobilised to the chip by two distinct methods, amine (semi-random) and thiol (physiological)-coupling, as described below.

For amine coupling, purified C3b (CompTech, A114) was immobilised, on a single flow cell of the CM5 chip on the BIAcore S200 (both GE) by flowing 5 μg/mL protein, diluted in 50 mM sodium acetate at pH 4.5, for several 20-second intervals at 20 μL/min, after the chip was activated by flowing ˜260 μL EDC and 180 μL NHS over the two flow cells to be used in experimentation (one for C3b binding and one blank), and until 800RU of C3b was immobilised to the appropriate flow cell. After this process ˜800RU of C3b was shown to be bound to the chip as displayed by the BIAcore sensorgram, shown in FIG. 2. This methodology was well described in Harris et al; Journal Biological Chemistry, 2005; 280(4):2569-78, and is in accordance with the manufacturer's instructions (NHS/EDC kit, GE). Any further active sites on the chip were blocked by an injection of 1M ethanolamine, for 2 minutes at 20 μL/min.

Alternatively, in order to couple C3b to the chip surface via its thioester domain, after immobilisation of a small amount of C3b by amine coupling as described previously (˜100RU), Factor B and Factor D (CompTech, A135 and A136 at 500 nM and 60 nM, respectively) were injected for 60s at 10 μL/min, in PBST or HBST containing 1 mM MgCl₂, to build the AP C3 convertase on the chip-bound C3b (C3bBb). Next, C3 (A114, CompTech, at 0.1 mg/mL) was injected immediately across the convertase for 180s at 10 μL/min, so that the C3 was cleaved to C3b by the chip-bound convertase. Rapid nucleophilic attack on the internal thioester resulted in covalent binding of the C3b to the surface through an ester bond at its TED domain. Several subsequent cycles of convertase formation and C3 cleavage resulted in ˜800-1000RU of C3b covalently immobilised on the chip surface in a physiologically relevant way.

In testing of a C3 variant, mutant (L1109V) and WT C3 were first activated by incubation with 2M methylamine (MA) at 1:10 (vMA:vC3) at 37° C. for 2 hrs. The C3MA was then buffer exchanged into PBS by the standard PD10 column (GE) procedure to remove excess free MA. It was ˜200RU of this C3MA that was bound to the chip surface by amine coupling before amplification through convertase building and injection of each of the respective C3 variants (using 90s injections of 500 nM CFB and 60 nM CFD followed immediately by 180s C3 injections at ˜0.1 mg/mL at 10 μL/min), WT and L1109V, on independent flow cells as displayed in FIG. 3. Eight-hundred and ninety-nine (899) RU of WT C3b and 898 RU of L1109V C3b were immobilised to the chip. Any further active sites on the chip were blocked by an injection of 1M ethanolamine, for 2 minutes at 20 μL/min.

### Example 2—Measuring Factor H CCP1-4 Variant Affinity to C3b by SPR on BIAcore S200

After size exclusion and buffer exchange, by gel filtration, of 6 FH1-4 variants into HEPES buffered saline with surfactant Tween 20 (HBST) (10 mM HEPES, 140 mM NaCl, 0.05% surfactant P20) or PBS, the preps were concentrated 10-fold using 10 kDa molecular weight cut-off Vivaspin columns (Sigma-Aldrich) and stored on ice or at −80° C. depending on when they were to be used. Each of the 6 variants (WT, P26S, R83S, T91S, R166W, R232Q) were flowed over both the blank flow cell and the C3b-immobilised flow cell (i.e., Fc1 to 2) at concentrations ranging from 20 μM to 0.3125 μM, diluted in HBST or PBST (PBS with 0.05% Tween 20), produced by a serial 1:2 dilution across a 96-well plate, for 90 seconds at a rate of 30 μL/min, with 120s allowed for dissociation. The flow cells were regenerated between each injection of an FH construct with an injection of 10 mM sodium acetate and 1 mM NaCl for 45 seconds at 20 μL/min. Data was collected at 40 Hz and there was a 120s stabilisation period between each sample being injected. Blank, buffer-only controls were included at several points during the automated run and RU changes on the blank flow cell (Fc1) were always subtracted from the C3b flow cell RU to give a normalised response. Steady state responses were plotted on the BIAevaluation software S200 (GE) as a function of time against RU for each concentration, before the same software was utilised to calculate affinity (K_{D}) of the variants to C3b (in μM) by plotting the concentration against RU from blank corrected traces to create an affinity curve, taking K_{D} as the concentration required to give 50% of RUmax for each variant, as shown in FIGS. 4 and 5.

### Example 3—FH1-4 Decay Acceleration Assay (DAA) Measured by SPR on BIAcore

In order to build the C3 convertase enzyme (C3bBb) on the CM5 chip, it was necessary to add 1 mM Mg₊ (in the form of MgCI2 to the HBST or PBST buffer previously utilised), both for diluting analyte and for general BIAcore flow. Buffer containing 1 mM Mg⁺, 500 nM Complement Factor B and 60 nM of Complement Factor D was injected across the chip for 90 seconds at 20 μL/min, building the C3 convertase, which was indicated by a relatively slow but large increase in RU. This complex was allowed to decay for 60 seconds as shown by a steady decrease in RU, before each FH1-4 variant, in addition to a blank buffer only control, was injected at several concentrations (500 nM serially diluted in buffer at a 1:2 ratio to 62.5 nM) for 60 seconds at 20 uL/min. On injection of functional FH1-4, a sharp drop in RU was seen in as Bb more rapidly dissociates from the surface complex—this is indicative of normal FH-induced decay acceleration activity, which is displayed in FIG. 6. Data was collected at 40 Hz and the flow cells were regenerated between each step with an injection of 10 mM Sodium Acetate and 1 mM NaCl for 40 seconds at 30 μL/min, after the end of each decay test, before the next injection of C3 convertase building buffer.

Each FH1-4 variant was compared to the WT and the nearest of several buffer-only injections performed after convertase building throughout each experiment.

For the analysis of DAA when comparing C3 variants, both FL-FH and FH1-4 were injected on to the chip, separately, across Fc 1 to 2 then Fc 3 to 4 in distinct cycles, at concentrations varying from 250 nM serially diluted 1:2 to 31.25 nM. When using FL-FH, due to the increase in affinity and subsequent signal produced by FL-FH binding to C3b even at low concentrations, FL-FH was injected separately onto the C3b-coupled chip with no prior injection of FD/FB, so that this signal could be subtracted from the sensorgram and give a real relative response curve.

### Example 4—On-Chip Alternate Pathway Regulatory TMC Building for Real-Time SPR Analysis of Variants in FH1-4, FI and C3 (C3b) Protein

A CM5 chip (GE) with ˜800RU C3b (CompTech, A114) immobilized by amine coupling, was used in the first instance to test the ability of WT FH1-4 to facilitate AP regulatory TMC formation with FI (CompTech, A138). At 4° C., WT FI was injected along with FH1-4, both at 5 μM (diluted in HBST₊Mg₊), for 60 seconds at 20 μL/min. After subtracting the RU given by the blank flow cell from the RU given by the C3b-immobilised flow cell to give an adjusted response, the AP regulatory TMC was shown to be built on the chip surface as displayed by a large RU spike indicating FI binding at high affinity to C3b:FH1-4 complexes. When using active WT FI at 4° C., cleavage of C3b to iC3b was seen as a gradual downward trend after the maximal binding point, before FH1-4 rapidly loses its affinity and dissociates in a fast-off (vertical) trend. After the event of the first successful TMC build using WT FI at 4° C., the chip surface was no longer stable and could not be used for further analysis of the FH:FI:C3b interaction, as was shown by a reduction in binding response (in RU) of FH and FI to the chip both individually and when injected together. No AP regulatory TMC appeared to be formed during this second attempt when FI and FH1-4 were injected simultaneously at the same (5 μM) concentration as previously. These phenomena are highlighted in FIG. 10.

Next, inactive FI (S525A) was utilised, at a constant (118 nM) concentration, injected with FH1-4 at four concentrations ranging from 118 nM to 14.75 nM by 3 serial 1:2 dilutions in PBST₊Mg⁺ (PBS with 0.05% Tween 20 and 1 mM MgCl₂) in a 96-well plate. Injections of FH1-4 (118-14.75 nM) and FI (118 nM) were made onto a new C3b-coupled CM5 chip (800RU, amine coupled) at the given concentrations for 2 mins at 30 μl/min, with a 500 second dissociation time. These injections were all performed in PBST₊Mg²⁺ buffer at 25° C. The flow cells were regenerated between each step with an injection of 10 mM sodium acetate and 1 mM NaCl for 40 seconds at 30 μL/min after the end of each test, before the next injection of TMC building component-containing buffer. Even at these low concentrations it was possible to see a large increase in RU on the C3b-coupled flow cell, before a slow decrease in RU after the injection stops, as FI and FH dissociate from the complex, as displayed in FIG. 7 (solid line).

To test for degradation of the chip surface, before and after the injections of FH1-4 variants with inactive (S525A) FI for comparative analysis, the C3 convertase (C3bBb) was built on the chip by injection of 500 nM FB and 60 nM FD for 90 seconds at 20 μL/min with a 90 second dissociation time, as previously, as displayed in FIG. 8 (convertase 1 and convertase 2). Additionally, FI and FH were injected, at 118 nM, onto the chip individually to check for normal fast on, fast off binding of both proteins to C3b, as displayed in FIG. 9. Further, to test AP regulatory TMC formation with active FI at 25° C., 118 nM of FH1-4 and 118 nM WT (active) FI were injected together and the chip was shown to be degraded almost instantaneously after formation of a small amount of TMC (˜3 RU) and conversion of C3b to iC3b, displayed in FIG. 8 (dashed line) alongside inactive (S525A) FI TMC building. As an additional and final test, FD and FB were added at standard convertase building concentrations and loss of C3b on the chip was be exemplified by the lower RU increase after injection of convertase components compared to before WT FI was used to cleave the chip surface, as shown in FIG. 8 (convertase 3). As always, sensorgrams were adjusted by subtracting the response given by injection of every test first over the appropriate blank flow cell.

If FI variants (with the additional inactive (S525A) change) were being tested on the AP regulatory TMC building assay, FI was serially diluted from 125 nM to 15.625 nM, whilst FH1-4 was kept at a constant concentration of 125 nM for each test injection and TMC build. Meanwhile, during testing of a C3 mutant (L1109V C3 vs WT C3), FL-FH (CompTech, A137) at concentrations ranging from 125 nM to 31.75 nM (from a 1:2 serial dilution) was injected along with constant concentrations of inactive (S525A) FI (at 125 nM). FL-FH injections were performed separately for the mutant and WT C3b (i.e., Fc 1 to 2, then Fc 3 to 4) on the same chip, as described when performing FL-FH affinity experiments.

### Results

### Example 5—Low Temperature AP Regulatory TMC Building

Attempts to develop a real-time assay of AP regulatory TMC formation using wild type, active FI at 4° C. faltered due to cleavage of C3b by WT active FI despite the low temperature (FIG. 10). Indeed, the injections in FIG. 10 display simple fast on/off binding of each protein (FH1-4 and FI) individually to C3b, as indicated by the sharp rise then fall in RU corresponding to the start and end of injection, respectively. However, when both proteins are injected together there is a large, rapid increase in RU representing the AP regulatory TMC followed by a gradual decay of the complex and finally a sharp drop in RU as the complex dissociates completely when the injection stops (labelled as ‘rapid FH dissociation’ in FIG. 10). The gradual degradation is marked as ‘C3b cleavage’ as it was hypothesised that during this stage FI is cleaving C3b into iC3b causing a loss of surface on the chip and loss of FH and FI affinity as C3f leaves the complex and the structure of iC3b is conformationally changed such that CCP4 of factor H loses its binding affinity. The following RU peaks are repeat injections of FI, FH1-4 before both are injected together for a second time. These data clearly show reduced binding of the proteins injected individually and in combination. The cleavage of the C3b substrate on the flow cell renders this assay impractical for repeated analysis of different variants because a separate flow cell would be required per repeat of AP regulatory TMC build at each concentration. The assay performed in the manner presented here would be expensive and possibly unreliable with slight differences in reactivity of the chip surfaces making intraassay comparisons difficult.

### Example 6—AP Regulatory TMC Building Using Inactive (S525A) FI at 25° C.

Inactive (S525A) FI has an amino acid change-S525A—that alters the nucleophile of its catalytic triad, rendering the enzyme ineffective at cleavage of C3b at any of its scissile bonds. S525A FI is sometimes referred to as S507A FI—its residue number given without the leader sequence. The position of S525A within the TMC can be seen in FIG. 11.

As shown in FIG. 12, injection of inactive (S525A) FI with WT FH1-4 (solid line) shows a gradual synergistic increase in binding followed by a slow dissociation with no indication of chip degradation, this was repeatable and was so until WT FI was introduced at the end of the experiment to cleave the chip surface. FB and FD injection before and after injection of the inactive (S525A) FI TMC results in a consistent level of response, whilst injection of FB and FD after active FI TMC building resulted in a substantially reduced response (FIG. 8). Inactive (S525A) FI therefore demonstrated binding of the components of the AP regulatory TMC without cleaving the chip surface.

In contrast to injections at 4° C., in FIG. 12 it is clear that WT active FI and FH1-4, when injected together onto a C3b-coupled CM5 chip at RT (dashed line), produce a curve showing a small but sharp increase in RU followed instantaneously by a gradual decline, which is indicative of loss of affinity of FH and FI to the chip surface and suggests that the surface is being cleaved instantly on injection and binding of FH and FI. Use of the inactive (S525A) FI resulted in it being possible to highly sensitively and reproducibly build an AP regulatory TMC on a C3b-coupled CM5 chip without the need to use multiple test surfaces. As is shown in the examples below, this enabled the evaluation of the effects of AMD-linked amino acid changes in FH, FI, and one aHUS-linked C3 variant, in terms of their efficacy in binding within the AP regulatory TMC.

### Example 7—Testing the Utility of the AP Regulatory TMC Building Assay in Analysis of Rare CFI, CFH and C3 Genetic Variants

### N-terminal CFH Variant Characterisation by AP Regulatory TMC Building

To test the utility of the AP regulatory TMC building assay, five FH1-4 variants were tested in their efficacy of formation of the TMC. Each variant (see FIG. 2) was injected along with inactive (S525A) FI across a C3b-thiol coupled chip and compared to a WT FH1-4 injection at various concentrations. The variants were then injected onto a C3b-amine coupled chip for further comparison of reproducibility. In these assays, the binding of the WT FH1-4 protein always resulted in the largest response, whilst T91S and P26S were efficient at forming a C3b:FH1-4:FI complex, with around 75% response compared to the WT. Conversely, R83S, R166W and R232Q appeared almost entirely unable to form the AP regulatory TMC on-chip, as is shown in FIG. 13 to 15, which is suggestive of an inability to bind either C3b or FI.

In order to show the assay yielded results that were repeatable, FH1-4 was injected at varying concentrations (118 nM serially diluted 1:2 to 28.5 nM), with inactive (S525A) FI remaining at a constant concentration. FIG. 14 shows that the curves representing injections of each FH CCP1-4 variant are reproducible across different concentrations. With similar differences between variants seen at each higher and lower concentration (118 nM and 28.5 nM, respectively) as there were in FIG. 12, which displayed AP regulatory TMC builds with each variant at 59 nM. 59 nM was used to compare between assays, when the method was repeated on a new surface that was amine-coupled rather than thiol-coupled.

FIG. 15 indicates that differences between the FH1-4 variants can be reproduced on an amine-coupled C3b chip surface, with similar AP regulatory TMC building efficacy differences between variants as was seen in FIG. 13 (on a thiol-coupled surface, also at 59 nM TMC), albeit with a lower overall response (in RU) on the amine coupled chip. Amine coupling of C3b results in a lower response because semi-random binding of the molecule renders some C3b bound in an orientation that blocks FH1-4 binding sites. Thiol coupling results in all the C3b molecules oriented so that all are able to bind FH1-4 and subsequently FI. The only discernible difference between the repeat on the amine-coupled chip was that there was more distinction between the performances of P26S and T91S FH1-4 variants, with T91S once again being the superior variant (as it was in affinity and decay experiments).

### FI Variant Characterisation by AP Regulatory TMC Building

The WT and Y459S FI variants were made on the inactive backbone; FI with only the S525A change (WT Inactive) and FI with both the Y459S and S525A changes (Y459S Inactive). The experiment was run once, with duplicate injections of 62.5 nM FI and 125 nM FH, as displayed in FIG. 16.

Using the method developed, it was possible to show that Y459S FI binding to FH/C3b was largely abrogated, with injection of the protein (at 62.5 nM) along with WT FH1-4 (at 125 nM) across a C3b-coupled chip resulting in only a small (˜2 RU) increase in response compared to the WT inactive FI protein, injection of which (at 62.5 nM) along with the FH1-4 (at 125 nM) resulted in a 15-20 RU increase in response. The lack of response is indicative of only a small amount of AP regulatory TMC being built on the chip surface with Y459S inactive FI, and therefore suggestive of Y459S having limited capability to bind FH and C3b within the AP regulatory TMC.

In addition, FI with only an H380R mutation was also constructed and binding to FH-1-4/C3b assessed (see FIG. 23). The experiment comprised injections of 125 nM of H380R FI and 125 nM FH1-4 onto a C3b amine coupled (˜1000RU) CM5 chip using the BIAcore S200, resulting in the building of an AP regulatory TMC (of ˜15RU). The complex did not immediately dissociate when the injection finished after 120 seconds. Thus, H380R FI appears to bind to WT FH1-4/C3b without cleaving the C3b coupled chip surface and therefore could be utilised instead of S525A as an inactivating mutation for additional variant characterisation. Meanwhile, injection of 125 nM of H380R FI alone onto the same surface resulted in only a ˜2RU response.

### C3 Variant Characterisation by AP Regulatory TMC Building

An aHUS patient in Newcastle-upon-Tyne, UK was discovered by the National Renal Complement Therapeutics Centre (NRCTC) as being homozygous for a rare genetic variant in the protein coding region of C3, L1109V (c.3325C>G:p.Leu1109Val). This provided an opportunity to show that the new AP regulatory TMC building method could be used to characterise a C3 genetic variant, and in doing so cover all 3 proteins of the TMC. Mutant and WT C3 protein were purified from human serum using standard liquid chromatography techniques described in detail in Ruseva et al; Totowa, NJ: Humana Press; 2014; 75-91. The mutant and WT C3 proteins were then activated by incubation with MA, which was used in the process of thiol-coupling C3b to a CM5 chip using the BIAcore S200, before affinity and TMC building experiments were performed. Inactive (S525A) FI was used for the AP regulatory TMC building experiments. As shown in FIG. 18, the C3b resulting from the C3 variant displayed a lack of binding to FH CCP19-20 (WT C3 K_{D}: 9.92 μM, mutant C3b K_{D}: 358 nM). The C-terminal domains of FH are those that associate with the thioester domain of C3b, which is the location of the mutated amino acid residue in this case, which is displayed in the 3D model in FIG. 19.

The variant C3b and WT C3b had a similar affinity to FH1-4 (WT C3b K_{D}: 8.1, Mutant C3b K_{D}: 9.2 μM). Therefore, it was hypothesised that the dysfunction was primarily in its binding to the C-terminus of FH during surface-phase regulation of the AP, which is common in aHUS, and would result in a lower avidity of C3b to FH as a whole. To confirm, FL-FH was injected onto each flow cell separately to calculate its estimate affinity to each C3b variant. Due to there being 2 binding sites on FL-FH to C3b, only an estimate could be made since the interaction is not steady state.

FL-FH had a 3.6-fold lower K_{D} to the mutant C3b compared to the WT C3b (L1109V K_{D}: 1.01M, WT C3b K_{D}: 0.28 μM). On inspection of the curves in FIG. 20, it appears that injection of FL-FH across the flow cell coupled with the mutant C3b resulted in binding similar to fast on/off in steady state kinetics, suggesting near total loss of the second binding site (at CCP19-20). Because the mutation primarily affected CCP19-20 binding, FL-FH was subsequently used to compare differences in AP regulatory TMC building efficacy rather than the in-house produced WT FH CCP1-4.

As shown in FIGS. 21 and 22, on injection of FL-FH and inactive (S525A) FI onto mutant and WT C3b-coupled chip surfaces, the WT C3b enabled far more TMC to bind, as represented by substantially higher RU peaks at each given concentration of FL-FH/FI injected. The maximum binding of the FH:FI to the WT C3b was 155RU, compared to 70RU bound to the mutant C3b.

These data show that the C3b produced by activation of the mutant C3 has a much lower (˜50%) propensity for forming the AP regulatory TMC compared to WT, which suggests that the rare L1109V C3b variant is resistant to breakdown by FI with FH acting as the cofactor.

Application of FHL-1, sMCP and Soluble CR1 in the AP Regulatory TMC Building Assay

In order to display that the full spectrum of cofactor proteins that can form part of the AP regulatory TMC could be used in the on-chip TMC building assay, FHL-1, sCR1 and sMCP were further injected with the inactive (S525A) FI. The FHL-1 used in this assay was produced in-house using standard CHO cell culturing. The sCR1 was produced as described in Rioux et al; Current Opinion on Investigational Drugs. 2001 March; 2(3):364-71. The sMCP was produced using E. coli, as described in Watson R., et al; Molecular Immunology, 2015, February; 63(2):287-96.

The different cofactors were injected onto the same C3b-coupled CM5 chip as was used to measure WT inactive (S525A) FI and Y459S inactive (S525A) FI TMC binding, and the WT inactive (S525) FI used was the same CHO-produced protein. The cofactors and inactive (S525A) FI were injected at 125 nM simultaneously using an identical method as previously, with the only difference being that FH1-4 was replaced by either FHL-1, sCR1 or sMCP, and the response was measured by SPR to give sensorgrams shown in FIGS. 24 and 25. Injections of cofactor without FI were made for comparison, as shown in FIGS. 26 and 27.

1.-32. (canceled)

33. A surface bound protein complex comprising:
a. a Complement C3b protein or variant thereof, or Complement C4b protein or a variant thereof,
b. a mutated Complement Factor I protein or variant thereof, and
c. a Complement Factor I co-factor protein,
wherein the Complement Factor I protein or variant thereof comprises at least one mutation that results in an inactivation or reduction of Complement Factor I protease activity.

34. A protein complex according to claim 33, wherein the protein complex comprises C3b, and further wherein the Complement Factor I cofactor protein is selected from:
d. a Complement Factor H protein or variant thereof, and/or
e. Factor-H-like protein 1 or variant thereof; and/or
f. soluble membrane cofactor protein (sMCP) or variant thereof, and/or
g. soluble complement receptor 1 (sCR1) or variant thereof.

35. A protein complex according to claim 33, wherein the protein complex comprises C4b, and further wherein the Complement Factor I cofactor protein is selected from:
h. soluble complement receptor 1 (sCR1) or variant thereof, and/or
i. C4b binding protein (C4 bp) or variant thereof, and/or
j. soluble membrane cofactor protein (sMCP) or variant thereof.

36. A protein complex according to claim 33, wherein: the mutation that results in inactivation or reduction of Complement Factor I protease activity is at amino acid position 380, and/or amino acid position 429, and/or amino acid position 525, wherein optionally the Complement Factor I protein comprises an S525A mutation and/or H380R mutation; and/or the mutation that results in inactivation or reduction of Complement Factor I protease activity is at amino acid position 380, and/or amino acid position 429, and/or amino acid position 525 according to the amino acid sequence as set forth in SEQ ID NO: 3

37. A protein complex according to claim 33, wherein the complex is bound to the surface of a surface plasmon resonance sensor chip, wherein optionally the sensor chip comprises a carboxymethyl group, further wherein optionally the complex is surface bound via an amine coupling, or a thiol coupling, or a coupling via a thioester.

38. A protein complex according to claim 33, wherein the Complement Factor I or variant thereof is a mammalian Complement Factor I protein, wherein optionally the mammalian Complement Factor I or variant thereof is a human Complement Factor I, further wherein optionally the human Complement Factor I protein comprises a first amino acid sequence selected from an amino acid sequence as set forth in SEQ ID NO: 4 (heavy chain) or an amino acid sequence having at least 85% sequence identity to the amino acid sequence as set forth in SEQ ID No: 4, and a second amino acid sequence as set forth in SEQ ID NO: 8 (light chain) or SEQ ID NO: 17 or an amino acid sequence having at least 85%, e.g. at least 90% sequence identity to the amino acid sequence set forth in SEQ ID NO: 8 or SEQ ID NO: 17, wherein the first and second amino acid sequences are linked via a disulphide bond.

39. A protein complex according to claim 34, wherein the Complement Factor H or variant thereof is a mammalian Complement Factor H protein, wherein optionally the mammalian Complement Factor H is a human Complement Factor H protein, further wherein optionally the human Complement Factor H protein comprises an amino acid sequence selected from an amino acid sequence as set forth in SEQ. ID. No. 1 or SEQ. ID. No. 2 or an amino acid sequence having at least 85%, e.g. 90% sequence identity to the amino acid sequence as set forth in SEQ. ID. No. 1 or SEQ. ID. No. 2.

40. A protein complex according to claim 33, wherein the C3b or variant thereof is a mammalian C3b, optionally wherein the mammalian C3b is a human C3b protein, further wherein optionally the human C3b protein comprises an amino acid sequence selected from an amino acid sequence as set forth in SEQ ID NO: 9 or an amino acid sequence having at least 85%, e.g. 90% sequence identity to the amino acid sequence as set forth in SEQ. ID. No. 9.

41. A protein complex according to claim 34, wherein the Factor-H-like protein 1 or variant thereof is a mammalian Factor-H-like protein 1, wherein optionally the mammalian Factor-H-like protein 1 is a human Factor-H-like protein 1, further wherein optionally the human Factor-H-like protein 1 comprises an amino acid sequence selected from an amino acid sequence as set forth in SEQ ID NO: 15 or an amino acid sequence having at least 85% sequence identity to the amino acid sequence as set forth in SEQ ID NO: 15.

42. A protein complex according to claim 34, wherein:
(A) the soluble membrane cofactor protein or variant thereof is a mammalian soluble membrane cofactor protein, wherein optionally the mammalian soluble membrane cofactor protein is a human soluble membrane cofactor protein, optionally further wherein the human soluble membrane cofactor protein comprises an amino acid sequence selected from an amino acid sequence as set forth in SEQ ID NO: 13 or an amino acid sequence having at least 85%, e.g. 90% sequence identity to the amino acid sequence as set forth in SEQ ID NO: 13; and/or
(B) the soluble complement receptor 1 protein or variant thereof is a mammalian soluble complement receptor 1 protein, wherein optionally the mammalian soluble complement receptor 1 protein is a human soluble complement receptor 1 protein, further wherein optionally the human soluble complement receptor 1 protein comprises an amino acid sequence selected from an amino acid sequence as set forth in SEQ ID NO: 11 or an amino acid sequence having at least 85%, e.g. 90% sequence identity to the amino acid sequence as set forth in SEQ ID NO: 11.

43. A method for determining the presence, absence or characteristics of a binding event associated with a protein complex, the protein complex comprising a mutant Complement Factor I protein or variant thereof, wherein the protein complex further comprises at least one of the following proteins:
k. a Complement C3b protein or variant thereof, or Complement C4b protein or a variant thereof, and
l. a Complement factor I cofactor protein,
wherein the Complement Factor I protein or variant thereof comprises at least one mutation that reduces or inactivates Complement Factor I protein protease activity of the Complement Factor I protein, wherein the method comprises:
i) forming said protein complex; and
ii) detecting the presence, absence and/or characteristics of a signal produced by a binding event associated with the formation of said protein complex, wherein said signal is produced by interaction of said Complement Factor I with said Complement C3b or variant thereof, or Complement C4b protein, and said Complement Factor I cofactor protein.

44. A method according to claim 43, which comprises forming a protein complex wherein the protein complex comprises:
(A) C3b, and further wherein the complement factor I cofactor protein is Complement Factor H protein or a variant thereof, or Factor-H-like protein 1 or a variant thereof, or soluble membrane cofactor protein (sMCP) or a variant thereof, or soluble complement receptor 1 (sCR1) or a variant thereof; or
(B) C4b, and further wherein the complement factor I cofactor is soluble complement receptor 1 (sCR1) or a variant thereof, or soluble membrane cofactor protein (sMCP) or a variant thereof, or C4b binding protein (C4 bp) or a variant thereof.

45. A method according to claim 43, wherein the method further comprises:
(A) prior to step (i), binding a component of said protein complex to a surface, wherein optionally the surface is a surface plasmon resonance sensor chip, further wherein the method optionally comprises binding a component of said protein complex to the surface via amine coupling, or thiol coupling, or a coupling via a thioester, wherein optionally the method comprises binding the Complement C3b protein or variant thereof, or Complement C4b protein or variant thereof to the surface; and/or
(B) detecting the presence or absence of a signal generated by a binding event associated with the formation of said protein complex using a sensor configured to detect said signal, wherein optionally the method further comprises detecting the presence, absence and/or characteristics of the binding event with a sensor configured to detect surface plasmon resonance.

46. A method according to claim 43, which comprises forming a protein complex wherein the mutation that reduces or inactivates Complement Factor I protein protease activity of the Complement Factor I protein is at amino acid position 380, and/or amino acid position 429, and/or amino acid position 525, wherein optionally the Complement Factor I protein comprises an S525A mutation and/or H380R mutation, wherein optionally the amino acid mutation at amino acid position 380, and/or amino acid position 429, and/or amino acid position 525 are positioned according to the amino acid sequence as set forth in SEQ. ID. No: 3.

47. A method according to claim 43, wherein the method further comprises: (A) determining the binding affinity of the interaction of said Complement Factor I with said Complement C3b or variant thereof, or Complement C4b or variant thereof, and said Complement Factor I cofactor protein; and/or (B) identifying a biomarker in a subject.

48. A method for determining the presence, absence, or characteristics of a ligand binding event associated with a protein complex comprising a mutant Complement Factor I protein or variant thereof, wherein the protein complex further comprises at least one of the following proteins;
m. a Complement C3b protein or variant thereof, or Complement C4b protein or a variant thereof, and
n. a Complement Factor I cofactor protein,
wherein the Complement factor I or variant thereof comprises at least one mutation that reduces or inactivates Complement Factor I protease activity of the Complement Factor I protein, and wherein the method comprises;
i) forming said protein complex, and
ii) contacting said protein complex with a candidate ligand molecule; and
iii) detecting the presence, absence, and/or characteristics of a signal generated by an interaction of said ligand molecule with said protein complex.

49. A method according to claim 48, wherein the method comprises:
(A) forming a protein complex wherein the Complement Factor I cofactor protein is selected from a Complement Factor H protein or variant thereof, a Factor-H-like protein 1 or variant thereof, a soluble membrane cofactor protein (sMCP) or variant thereof, a C4b binding protein (C4 bp) or variant thereof, and a soluble complement receptor 1 (sCR1) or variant thereof; and/or
(B) prior to step (i) binding a component of the protein complex to a surface, wherein optionally the surface is a surface plasmon resonance sensor chip, wherein optionally the method comprises binding a component of the protein complex to said surface via amine coupling, or thiol coupling, or a coupling via a thioester, and further wherein optionally the method comprises binding the Complement C3b protein or variant thereof, or Complement C4b protein or variant thereof to the surface; and/or
(C) detection of the presence, absence and/or characteristic of a signal generated by an interaction of said candidate ligand molecule with said protein complex using a sensor configured to detect said signal, wherein optionally the method comprises using a sensor configured to detect surface plasmon resonance.

50. A method according to claim 48, which further comprises:
(A) determining a binding affinity of said ligand molecule with said protein complex; and/or
(B) identifying a biomarker in a subject.

51. A surface comprising a complex according to claim 33.
